(19) United States
(12) Patent Application Publication
(10) Pub. No.: US 2018/0110871 A1
(43) Pub. Date: Apr. 26, 2018

(54) DUAL DISULFIDE DRUG CONJUGATES

(71) Applicant: ENDOCYTE, INC., West Lafayette, IN (US)

(72) Inventors: Iontcho Radoslavov VLAHOV, West Lafayette, IN (US); Christopher Paul LEAMON, West Lafayette, IN (US); Fei YOU, West Lafayette, IN (US); Hanna Francesca KLEIN, Bedford, Bedfordshire (GB)

(21) Appl. No.: 15/566,405

(22) PCT Filed: Apr. 14, 2016
(86) PCT No.: PCT/US16/27547

§ 371 Date: Oct. 13, 2017

Related U.S. Application Data
(60) Provisional application No. 62/149,212, filed on Apr. 17, 2015.

(51) Int. Cl. A61K 47/65; A61K 38/05; A61K 47/54; A61P 35/00

(52) U.S. Cl. CPC A61K 47/65; A61P 35/00; A61K 47/545; A61K 38/05

(57) ABSTRACT

The invention described herein pertains to dual disulfide drug conjugates. In particular, the invention described herein pertains to dual disulfide drug conjugates that target the folate receptor for delivery of conjugated drugs to a mammalian recipient. Also described are methods of making and using dual disulfide drug conjugates.

### CROSS REFERENCE TO RELATED APPLICATIONS

This application claims priority under 35 U.S.C. § 119(e) to U.S. Provisional Application Ser. No. 62/149,212, filed Apr. 17, 2015, which is incorporated herein by reference in its entirety.

### TECHNICAL FIELD

The invention described herein pertains to dual disulfide drug conjugates. In particular, the invention described herein pertains to dual disulfide drug conjugates that target the folate receptor for delivery of conjugated drugs to a mammalian recipient. Also described are methods of making and using dual disulfide drug conjugates.

### BACKGROUND

The mammalian immune system provides a means for the recognition and elimination of pathogenic cells, such as tumor cells, cancers, and other invading foreign pathogens. While the immune system normally provides a strong line of defense, there are many instances where pathogenic cells, such as cancer cells, and other infectious agents evade a host immune response and proliferate or persist with concomitant host pathogenicity. Chemotherapeutic agents, radiation therapies, and hormone therapy have been developed to eliminate, for example, replicating neoplasms. Despite the significant developments in anti-cancer technology, cancer still remains the second leading cause of death following heart disease in the United States. Most often, cancer is treated with radiation therapy and/or chemotherapy utilizing highly potent drugs, such as mitomycin, paclitaxel and camptothecin. However, radiation therapy regimens have adverse side effects because they lack sufficient selectivity to preferentially destroy pathogenic cells, and therefore, may also harm normal host cells, such as cells of the hematopoietic system, and other non-pathogenic cells. Though chemotherapeutic agents show a dose responsive effect, and cell kill is proportional to drug dose, a highly aggressive style of dosing is generally necessary to eradicate neoplasms. Such high-dose chemotherapy is often compromised by poor selectivity for cancer cells and severe toxicity to normal cells. Adverse side effects and the lack of tumor-specific treatment using many current therapies highlight the need for the development of new therapies selective for treating cancers with reduced host toxicity.

Membrane transport of antifolate therapeutics, such as methotrexate, has found application in the treatment of a variety of malignancies and nonmalignant diseases. The major membrane transporters include the reduced folate carrier (RFC), the proton-coupled folate transporter (PCFT), and the high affinity folate receptors (FRs) a and (3. Whereas both RFC and PCFT are integral membrane proteins that act as facilitative transporters, FRs are glycosyl phosphatidylinositol-modified proteins that mediate cellular uptake of (anti)folates by receptor-mediated endocytosis.

The major folate transporters also differ in terms of their tissue distributions. For example, RFC is ubiquitously expressed in tumors and tissues and is the primary uptake mechanism for folate cofactors. FRs are known to be expressed in certain malignancies, such as the FRα isoform in ovarian carcinomas, and in some normal epithelial tissues such as renal tubules. Major sites of PCFT expression include the upper small intestine (e.g., jejunum) and the liver and kidney.

In solid tumors such as hepatomas, ovarian carcinomas, and non-small-cell lung carcinomas, PCFT is highly expressed. PCFT exhibits an acidic pH optimum, which is compatible with the low pH microenvironments of the small intestine and many solid tumors. While PCFT is modestly expressed in most other normal tissues, for those in which PCFT is expressed they are unlikely to present the low pH conditions optimal for membrane transport by this mechanism.

Folic acid (FA) hinds with high affinity (K_{D}<10−9 M) to folate receptor (FR)-α glycosylphosphatidylinositol anchored cell-surface glycoprotein. After binding, FA is transported into the cell via FR-mediated endocytosis.

It has been discovered that drugs can be targeted to cancer cells, tissues, and tumors using antifolates. Described herein are conjugates and compositions, and associated methods and uses for treating cancer.

### SUMMARY

In one aspect, the disclosure provides conjugates of the formula B-L-D¹, wherein B is a binding ligand, L is a linker comprising at least two L¹ as described herein and D¹ is a drug, wherein B and D¹ are defined as described herein in various embodiments and examples.

In another aspect, the disclosure provides pharmaceutical compositions comprising a therapeutically effective amount of the conjugates described herein, or a pharmaceutically acceptable salt thereof, and at least on excipient.

In another aspect, the disclosure provides a method of treating abnormal cell growth in a mammal, including a human, the method comprising administering to the mammal any of the conjugates or compositions described herein.

The conjugates of the present disclosure can be described as embodiments in any of the following enumerated clauses. It will be understood that any of the embodiments described herein can be used in connection with any other embodiments described herein to the extent that the embodiments do not contradict one another.

In some embodiments, some conjugates described herein are of the formula

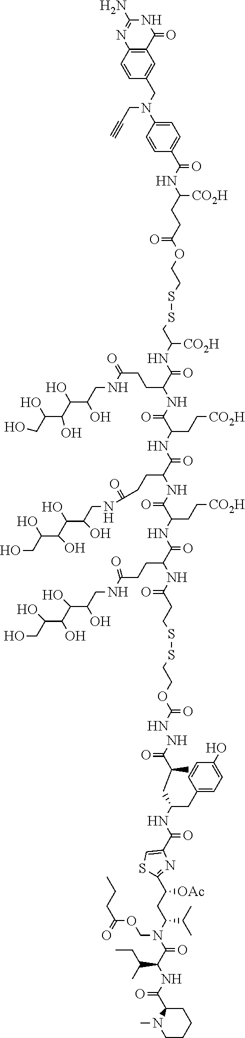 [SMILES: C#CCN(Cc1ccc2nc(N)[nH]c(=O)c2c1)c1ccc(C(=O)NC(CCC(=O)OCCSSCC(NC(=O)C(CCC(=O)NCC(O)C(O)C(O)C(O)CO)NC(=O)C(CCC(=O)O)NC(=O)C(CCC(=O)NCC(O)C(O)C(O)C(O)CO)NC(=O)C(CCC(=O)O)NC(=O)C(CCC(=O)NCC(O)C(O)C(O)C(O)CO)NC(=O)CCSSCCOC(=O)NNC(=O)[C@@H](C)C[C@H](Cc2ccc(O)cc2)NC(=O)c2csc([C@@H](C[C@H](C(C)C)N(COC(=O)CCC)C(=O)[C@@H](NC(=O)[C@H]3CCCCN3C)C(C)CC)OC(C)=O)n2)C(=O)O)C(=O)O)cc1]

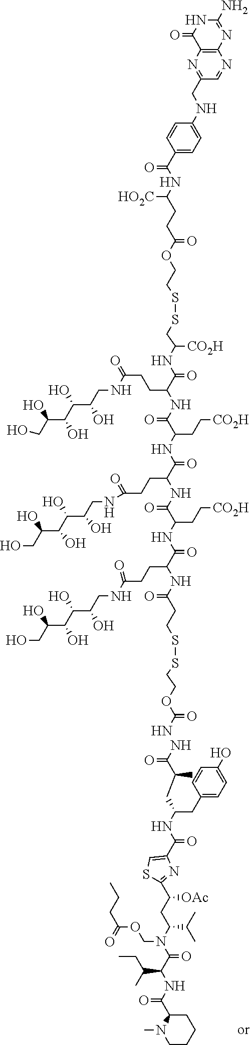 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSCCC(=O)NC(CCC(=O)NC[C@H](O)[C@@H](O)[C@H](O)[C@H](O)CO)C(=O)NC(CCC(=O)O)C(=O)NC(CCC(=O)NC[C@H](O)[C@@H](O)[C@H](O)[C@H](O)CO)C(=O)NC(CCC(=O)O)C(=O)NC(CCC(=O)NC[C@H](O)[C@@H](O)[C@H](O)[C@H](O)CO)C(=O)NC(CSSCCOC(=O)CCC(NC(=O)c2ccc(NCc3cnc4nc(N)[nH]c(=O)c4n3)cc2)C(=O)O)C(=O)O)cs1)C(C)C]

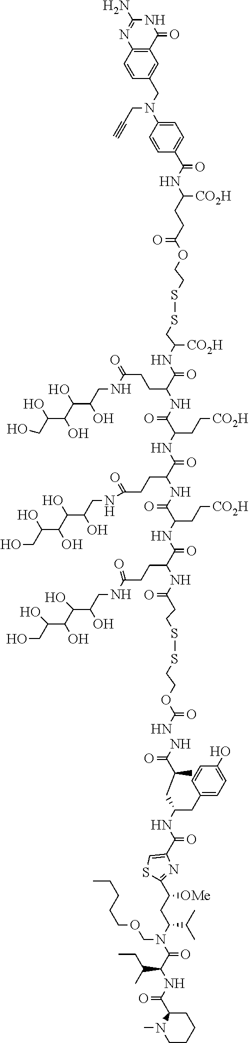 [SMILES: C#CCN(Cc1ccc2nc(N)[nH]c(=O)c2c1)c1ccc(C(=O)NC(CCC(=O)OCCSSCC(NC(=O)C(CCC(=O)NCC(O)C(O)C(O)C(O)CO)NC(=O)C(CCC(=O)O)NC(=O)C(CCC(=O)NCC(O)C(O)C(O)C(O)CO)NC(=O)C(CCC(=O)O)NC(=O)C(CCC(=O)NCC(O)C(O)C(O)C(O)CO)NC(=O)CCSSCCOC(=O)NNC(=O)[C@@H](C)C[C@H](Cc2ccc(O)cc2)NC(=O)c2csc([C@@H](C[C@H](C(C)C)N(COCCCCC)C(=O)[C@@H](NC(=O)[C@H]3CCCCN3C)C(C)CC)OC)n2)C(=O)O)C(=O)O)cc1]

or a pharmaceutically acceptable salt thereof.

In some embodiments, some conjugates described herein are of the formula

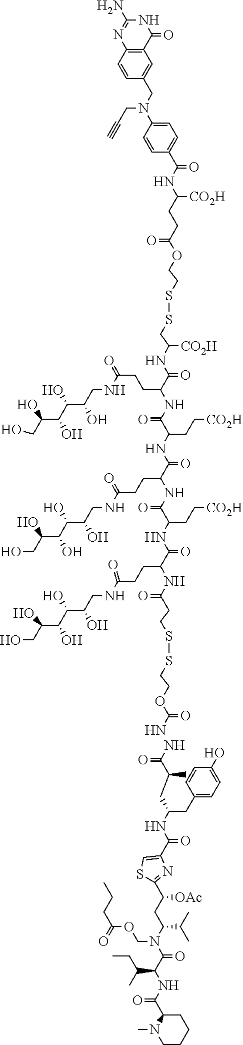 [SMILES: C#CCN(Cc1ccc2nc(N)[nH]c(=O)c2c1)c1ccc(C(=O)NC(CCC(=O)OCCSSCC(NC(=O)C(CCC(=O)NC[C@H](O)[C@@H](O)[C@H](O)[C@H](O)CO)NC(=O)C(CCC(=O)O)NC(=O)C(CCC(=O)NC[C@H](O)[C@@H](O)[C@H](O)[C@H](O)CO)NC(=O)C(CCC(=O)O)NC(=O)C(CCC(=O)NC[C@H](O)[C@@H](O)[C@H](O)[C@H](O)CO)NC(=O)CCSSCCOC(=O)NNC(=O)[C@@H](C)C[C@H](Cc2ccc(O)cc2)NC(=O)c2csc([C@@H](C[C@H](C(C)C)N(COC(=O)CCC)C(=O)[C@@H](NC(=O)[C@H]3CCCCN3C)C(C)CC)OC(C)=O)n2)C(=O)O)C(=O)O)cc1]

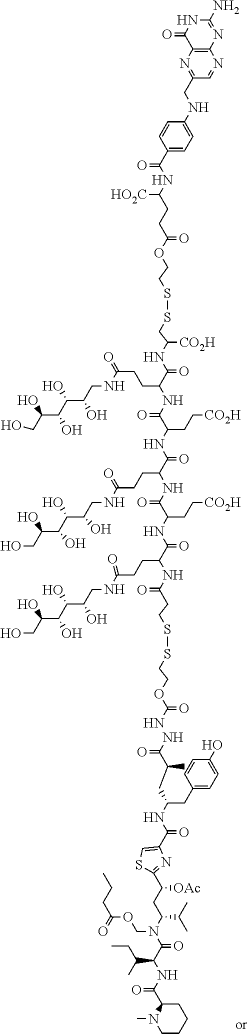 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSCCC(=O)NC(CCC(=O)NC[C@H](O)[C@@H](O)[C@H](O)[C@H](O)CO)C(=O)NC(CCC(=O)O)C(=O)NC(CCC(=O)NC[C@H](O)[C@@H](O)[C@H](O)[C@H](O)CO)C(=O)NC(CCC(=O)O)C(=O)NC(CCC(=O)NC[C@H](O)[C@@H](O)[C@H](O)[C@H](O)CO)C(=O)NC(CSSCCOC(=O)CCC(NC(=O)c2ccc(NCc3cnc4nc(N)[nH]c(=O)c4n3)cc2)C(=O)O)C(=O)O)cs1)C(C)C]

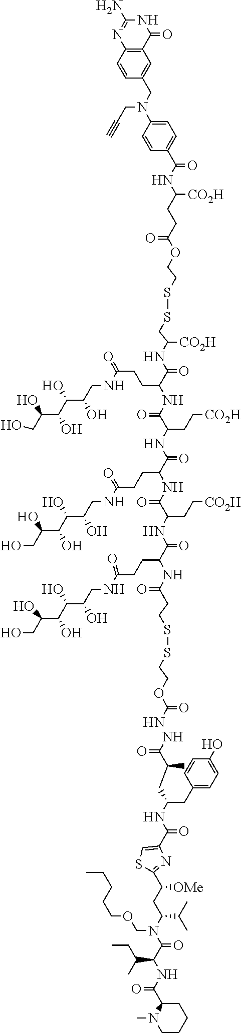 [SMILES: C#CCN(Cc1ccc2nc(N)[nH]c(=O)c2c1)c1ccc(C(=O)NC(CCC(=O)OCCSSCC(NC(=O)C(CCC(=O)NC[C@H](O)[C@@H](O)[C@H](O)[C@H](O)CO)NC(=O)C(CCC(=O)O)NC(=O)C(CCC(=O)NC[C@H](O)[C@@H](O)[C@H](O)[C@H](O)CO)NC(=O)C(CCC(=O)O)NC(=O)C(CCC(=O)NC[C@H](O)[C@@H](O)[C@H](O)[C@H](O)CO)NC(=O)CCSSCCOC(=O)NNC(=O)[C@@H](C)C[C@H](Cc2ccc(O)cc2)NC(=O)c2csc([C@@H](C[C@H](C(C)C)N(COCCCCC)C(=O)[C@@H](NC(=O)[C@H]3CCCCN3C)C(C)CC)OC)n2)C(=O)O)C(=O)O)cc1]

or a pharmaceutically acceptable salt thereof.

In some embodiments, the disclosure provides a conjugate selected from the group consisting of

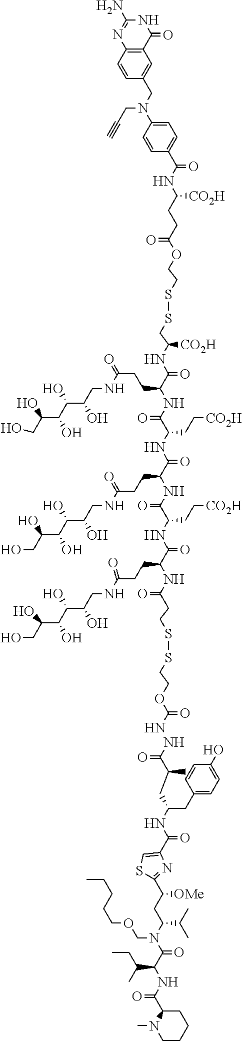 [SMILES: C#CCN(Cc1ccc2nc(N)[nH]c(=O)c2c1)c1ccc(C(=O)N[C@@H](CCC(=O)OCCSSC[C@H](NC(=O)[C@H](CCC(=O)NC[C@H](O)[C@@H](O)[C@H](O)[C@H](O)CO)NC(=O)[C@H](CCC(=O)O)NC(=O)[C@H](CCC(=O)NC[C@H](O)[C@@H](O)[C@H](O)[C@H](O)CO)NC(=O)[C@H](CCC(=O)O)NC(=O)[C@H](CCC(=O)NC[C@H](O)[C@@H](O)[C@H](O)[C@H](O)CO)NC(=O)CCSSCCOC(=O)NNC(=O)[C@@H](C)C[C@H](Cc2ccc(O)cc2)NC(=O)c2csc([C@@H](C[C@H](C(C)C)N(COCCCCC)C(=O)[C@@H](NC(=O)[C@H]3CCCCN3C)C(C)CC)OC)n2)C(=O)O)C(=O)O)cc1]

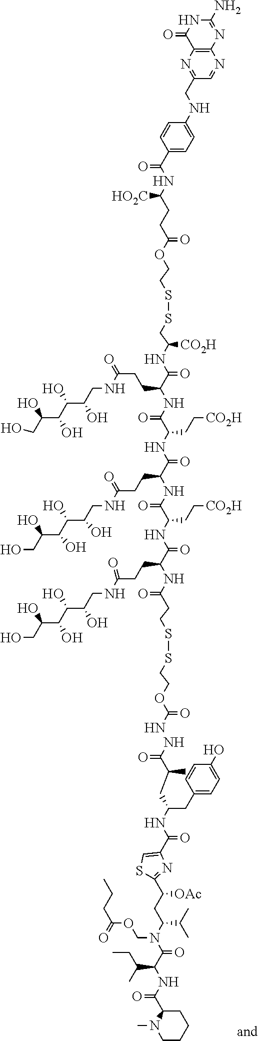 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSCCC(=O)N[C@@H](CCC(=O)NC[C@H](O)[C@@H](O)[C@H](O)[C@H](O)CO)C(=O)N[C@@H](CCC(=O)O)C(=O)N[C@@H](CCC(=O)NC[C@H](O)[C@@H](O)[C@H](O)[C@H](O)CO)C(=O)N[C@@H](CCC(=O)O)C(=O)N[C@@H](CCC(=O)NC[C@H](O)[C@@H](O)[C@H](O)[C@H](O)CO)C(=O)N[C@@H](CSSCCOC(=O)CC[C@H](NC(=O)c2ccc(NCc3cnc4nc(N)[nH]c(=O)c4n3)cc2)C(=O)O)C(=O)O)cs1)C(C)C]

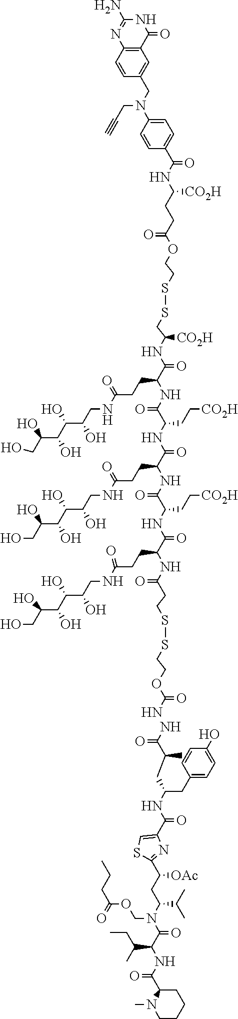 [SMILES: C#CCN(Cc1ccc2nc(N)[nH]c(=O)c2c1)c1ccc(C(=O)N[C@@H](CCC(=O)OCCSSC[C@H](NC(=O)[C@H](CCC(=O)NC[C@H](O)[C@@H](O)[C@H](O)[C@H](O)CO)NC(=O)[C@H](CCC(=O)O)NC(=O)[C@H](CCC(=O)NC[C@H](O)[C@@H](O)[C@H](O)[C@H](O)CO)NC(=O)[C@H](CCC(=O)O)NC(=O)[C@H](CCC(=O)NC[C@H](O)[C@@H](O)[C@H](O)[C@H](O)CO)NC(=O)CCSSCCOC(=O)NNC(=O)[C@@H](C)C[C@H](Cc2ccc(O)cc2)NC(=O)c2csc([C@@H](C[C@H](C(C)C)N(COC(=O)CCC)C(=O)[C@@H](NC(=O)[C@H]3CCCCN3C)C(C)CC)OC(C)=O)n2)C(=O)O)C(=O)O)cc1]

1. A conjugate of the formula B-L-D¹, wherein B is a binding ligand, L is a linker comprising at least two L¹, at least one AA, and at least one L² of the formula

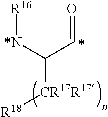

wherein

R¹⁶ is selected from the group consisting of H, D, C₁-C₆ alkyl, C₂-C₆ alkenyl, C₂-C₆ alkynyl, —C(O)R¹⁹, —C(O)OR¹⁹ and —C(O)NR¹⁹R^{19′}, wherein each hydrogen atom in C₁-C₆ alkyl, C₂-C₆ alkenyl and C₂-C₆ alkynyl is independently optionally substituted by halogen, C₁-C₆ alkyl, C₂-C₆ alkenyl, and C₂-C₆ alkynyl, —OR²⁰, —OC(O)R²⁰, —OC(O)NR²⁰R^{20′}, —OS(O)R²⁰, —OS(O)₂R²⁰, —SR²⁰, —S(O)R²⁰, —S(O)₂R²⁰, —S(O)NR²⁰R^{20′}, —S(O)₂NR²⁰R^{20′}, —OS(O)NR²⁰R^{20′}, —OS(O)₂NR²⁰R^{20′}, —NR²⁰R^{20′}, —NR²⁰C(O)R²¹, —NR²⁰C(O)OR²¹, —NR²⁰C(O)NR²¹R^{21′}, —NR²⁰S(O)R²¹, —NR²⁰S(O)₂R²¹, —NR²⁰S(O)NR²¹R^{21′}, —NR²⁰S(O)₂NR²¹R^{21′}, —C(O)R²⁰, —C(O)OR²⁰ or —C(O)NR²⁰R^{20′};

each R¹⁷ and R^{17′} is independently selected from the group consisting of H, D, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²², —OC(O)R²², —OC(O)NR²²R^{22′}, —OS(O)R²², —OS(O)₂R²², —SR²², —S(O)R²², —S(O)₂R²², —S(O)NR²²R^{22′}, —S(O)₂NR²²R^{22′}, —OS(O)NR²²R^{22′}, —OS(O)₂NR²²R^{22′}, —NR²²R^{22′}, —NR²²C(O)R²³, —NR²²C(O)OR²³, —NR²²C(O)NR²³R^{23′}, —NR²²S(O)R²³, —NR²²S(O)₂R²³, —NR²²S(O)NR²³R^{23′}, —NR²²S(O)₂NR²³R^{23′}, —C(O)R²², —C(O)OR²², and —C(O)NR²²R^{22′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, C₂-C₆ alkynyl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′}; or R¹⁷ and R^{17′} may combine to form a C₄-C₆ cycloalkyl or a 4- to 6-membered heterocycle, wherein each hydrogen atom in C₄-C₆ cycloalkyl or 4- to 6-membered heterocycle is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′};

R¹⁸ is selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26″})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};

each R¹⁹, R^{19′}, R²⁰, R^{20′}, R²¹, R^{21′}, R²², R^{22′}, R²³, R^{23′}, R²⁴, R^{24′}, R²⁵, R^{25′}, R²⁶, R^{26′}, R^{26″}, R²⁹, R^{29′}, R³⁰ and R^{30′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;

R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}— (sugar) and —(CH₂)ₚ(OCH₂CH₂CH₂)_{q}(sugar);

R²⁸ is H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;

n is 1, 2, 3, 4 or 5;

p is 1, 2, 3, 4 or 5;

q is 1, 2, 3, 4 or 5;

L¹ is a releasable linker comprising a disulfide moiety;

D¹ is a drug; and

each * is a covalent bond;

or a pharmaceutically acceptable salt thereof,

with the proviso that the conjugate is not of the formula

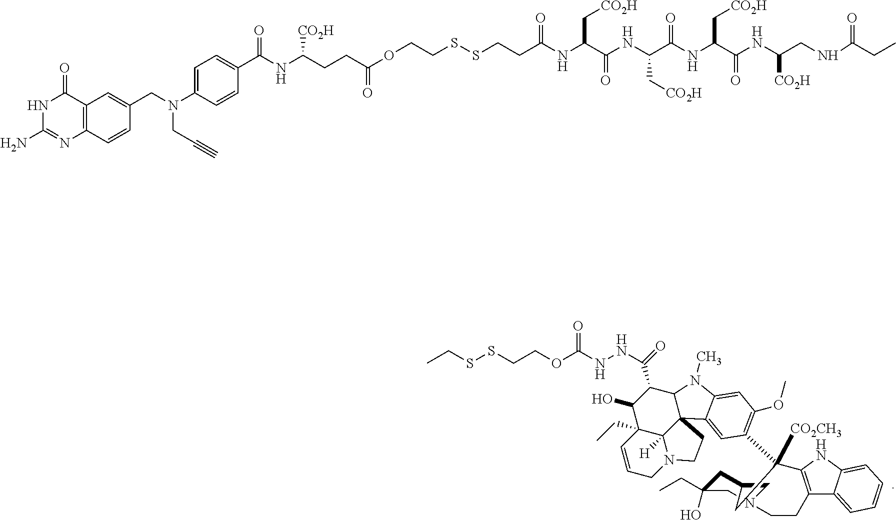

2. The conjugate of clause 1, wherein B is of the formula I

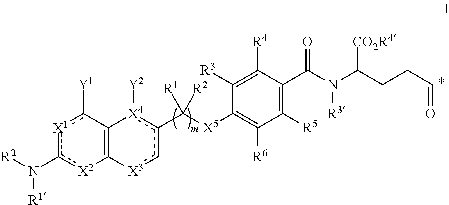

wherein

R¹ and R² in each instance are independently selected from the group consisting of H, D, halogen, C₁-C₆ alkyl, C₂-C₆ alkenyl, C₂-C₆ alkynyl, —OR⁷, —SR⁷ and —NR⁷R^{7′}, wherein each hydrogen atom in C₁-C₆ alkyl, C₂-C₆ alkenyl and C₂-C₆ alkynyl is independently optionally substituted by halogen, —OR⁸, —SR⁸, —NR⁸R^{8′}, —C(O)R⁸, —C(O)OR⁸ or —C(O)NR⁸R^{8′};

R³, R⁴, R⁵ and R⁶ are each independently selected from the group consisting of H, D, halogen, C₁-C₆ alkyl, C₂-C₆ alkenyl, C₂-C₆ alkynyl, —CN, —NO₂, —NCO, —OR⁹, —SR⁹, —NR⁹R^{9′}, —C(O)R⁹, —C(O)OR⁹ and —C(O)NR⁹R^{9′}, wherein each hydrogen atom in C₁-C₆ alkyl, C₂-C₆ alkenyl and C₂-C₆ alkynyl is independently optionally substituted by halogen, —OR¹⁰, —SR¹⁰, —NR¹⁰R^{10′}, —C(O)R¹⁰, —C(O)OR¹⁰ or —C(O)NR¹⁰R^{10′};

each R⁷, R^{7′}, R⁸, R^{8′}, R⁹, R^{9′}, R¹⁰ and R^{10′} is independently H, D, C₁-C₆ alkyl, C₂-C₆ alkenyl or C₂-C₆ alkynyl;

X¹ is NR¹¹—, ═N—, —N═, —C(R¹¹)═ or ═C(R¹¹)—;

X² is NR^{11′}- or ═N—;

X³ is NR^{11″}—, —N═ or —C(R^{11′})=;

X⁴ is N═ or C═;

X⁵ is —NR¹²— or —CR¹²R^{12′}—;

Y¹ is H, D, —OR¹³, —SR¹³ or —NR¹³R^{13′} when X¹ is —N═ or —C(R¹¹)═, or Y¹ is ═O when X¹ is NR¹¹—, ═N— or ═C(R¹¹)—;

Y² is H, D, C₁-C₆ alkyl, C₂-C₆ alkenyl, —C(O)R¹⁴, —C(O)OR¹⁴ or —C(O)NR¹⁴R^{14′} when X⁴ is —C═, or Y² is absent when X⁴ is N═;

R^{1′}, R^{2′}, R^{3′}, R^{4′}, R¹¹, R^{11′}, R^{11″}, R¹², R^{12′}, R¹³, R^{13′}, R¹⁴ and R^{14′} are each independently selected from the group consisting of H, D, C₁-C₆ alkyl, C₂-C₆ alkenyl, C₂-C₆ alkynyl, —C(O)R¹⁵, —C(O)OR¹⁵ and —C(O)NR¹⁵R^{15′};

R¹⁵ and R^{15′} are each independently H or C₁-C₆ alkyl; and

m is 1, 2, 3 or 4;

or a pharmaceutically acceptable salt thereof.

3. The conjugate of clause 1 or 2 of the formula

B-L¹-L²-AA-L²-AA-L²-L¹-D¹

or a pharmaceutically acceptable salt thereof.

4. The conjugate of any one of clauses 2 or 3, or a pharmaceutically acceptable salt thereof, wherein m is 1.

5. The conjugate of any one of clauses 2 to 4, or a pharmaceutically acceptable salt thereof, wherein X¹ is NR¹¹—.

6. The conjugate of any one of clauses 2 to 5, or a pharmaceutically acceptable salt thereof, wherein X² is ═N—.

7. The conjugate of any one of clauses 2 to 6, or a pharmaceutically acceptable salt thereof, wherein Y¹ is ═O.

8. The conjugate of any one of clauses 2 to 7, or a pharmaceutically acceptable salt thereof, wherein X¹ is NR¹¹—, and R¹¹ is H.

9. The conjugate of any one of clauses 2 to 8, or a pharmaceutically acceptable salt thereof, wherein X³ is —C(R^{11′})=.

10. The conjugate of clause 9, or a pharmaceutically acceptable salt thereof, wherein R^{11′} is H.

11. The conjugate of any one of clauses 2 to 10, or a pharmaceutically acceptable salt thereof, wherein X⁴ is —C═.

12. The conjugate of any one of clauses 2 to 11, or a pharmaceutically acceptable salt thereof, wherein Y² is H.

13. The conjugate of any one of clauses 2 to 8, or a pharmaceutically acceptable salt thereof, wherein X³ is —N═.

14. The conjugate of any one of clauses 2 to 8 or 13, or a pharmaceutically acceptable salt thereof, wherein X⁴ is N═.

15. The conjugate of any one of clauses 2 to 14, or a pharmaceutically acceptable salt thereof, wherein X⁵ is NR¹²—.

16. The conjugate of any one of clauses 2 to 15, or a pharmaceutically acceptable salt thereof, wherein R¹² is C₂-C₆ alkynyl.

17. The conjugate of any one of clauses 2 to 16, or a pharmaceutically acceptable salt thereof, wherein R¹² is propyn-3-yl.

18. The conjugate of any one of clauses 2 to 17, or a pharmaceutically acceptable salt thereof, wherein R¹² is H.

19. The conjugate of any one of clauses 2 to 18, or a pharmaceutically acceptable salt thereof, wherein R^{1′} and R^{2′} are H.

20. The conjugate of any one of clauses 2 to 19, or a pharmaceutically acceptable salt thereof, wherein R^{3′} is H.

21. The conjugate of any one of clauses 2 to 19, or a pharmaceutically acceptable salt thereof, wherein R^{4′} is H.

22. The conjugate of any one of clauses 2 to 20, or a pharmaceutically acceptable salt thereof, wherein each R¹ and R² is H.

23. The conjugate of any one of clauses 2 to 20, or a pharmaceutically acceptable salt thereof, wherein R³, R⁴, R⁵ and R⁶ are H.

24. The conjugate of clause 2, or a pharmaceutically acceptable salt thereof, wherein B is of the formula

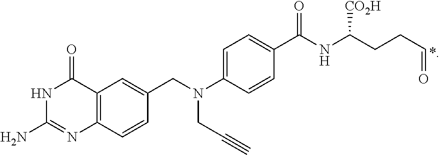 [SMILES: C#CCN(Cc1ccc2nc(N)[nH]c(=O)c2c1)c1ccc(C(=O)N[C@@H](CCC=O)C(=O)O)cc1]

25. The conjugate of clause 2, or a pharmaceutically acceptable salt thereof, wherein B is of the formula

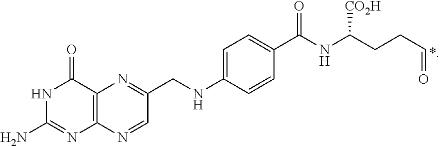 [SMILES: Nc1nc2ncc(CNc3ccc(C(=O)N[C@@H](CCC=O)C(=O)O)cc3)nc2c(=O)[nH]1]

26. The conjugate of any one of clauses 1 to 25, or a pharmaceutically acceptable salt thereof, wherein at least one AA is in the D-configuration.

27. The conjugate of any one of clauses 1 to 26, or a pharmaceutically acceptable salt thereof, wherein at least one AA is in the L-configuration.

28. The conjugate of any one of clauses 1 to 25, or a pharmaceutically acceptable salt thereof, wherein at least one AA is selected from the group consisting of L-asparagine, L-arginine, L-glycine, L-aspartic acid, L-glutamic acid, L-glutamine, L-cysteine, L-alanine, L-valine, L-leucine, L-isoleucine, L-citrulline, D-asparagine, D-arginine, D-glycine, D-aspartic acid, D-glutamic acid, D-glutamine, D-cysteine, D-alanine, D-valine, D-leucine, D-isoleucine and D-citrulline.

29. The conjugate of any one of clauses 1 to 28, or a pharmaceutically acceptable salt thereof, wherein at least one AA is selected from the group consisting of Asp, Arg, Val, Ala, Cys and Glu.

30. The conjugate of any one of clauses 1 to 29, wherein each L¹ is selected from the group consisting of

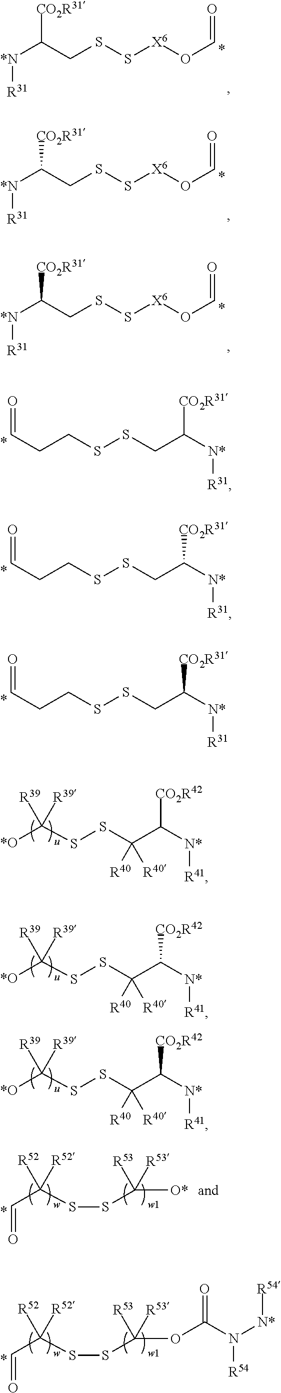

wherein

each R³¹ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};

each R^{31′} is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR^{32a}, —OC(O)R^{32a}, —OC(O)NR^{32a}R^{32a′}, —OS(O)R^{32a}, —OS(O)₂R^{32a}, —SR^{32a}, —S(O)R^{32a}, —S(O)₂R^{32a}, —S(O)NR^{32a}R^{32a′}, —S(O)₂NR^{32a}R^{32a′}, —OS(O)NR^{32a}R^{32a′}, —OS(O)₂NR^{32a}R^{32a′}, —NR^{32a}R^{32a′}, —C(O)R^{32a}, —C(O)OR^{32a} or —C(O)NR^{32a}R^{32a′};

each X⁶ is independently C₁-C₆ alkyl or C₆-C₁₀ aryl(C₁-C₆ alkyl), wherein each hydrogen atom in C₁-C₆ alkyl and C₆-C₁₀ aryl(C₁-C₆ alkyl) is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —OS(O)₂R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —OS(O)₂NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴, —C(O)OR³⁴ or —C(O)NR³⁴R^{34′};

each R^{32a}, R^{32a′}, R³², R^{32′}, R³³, R^{33′}, R³⁴, R^{34′}, R³⁵ and R^{35′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl;

each R³⁹, R^{39′}, R⁴⁰ and R^{40′} is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁴⁴, —OC(O)R⁴⁴, —OC(O)NR⁴⁴R^{44′}, —OS(O)R⁴⁴, —OS(O)₂R⁴⁴, —SR′, —S(O)R⁴⁴, —S(O)₂R⁴⁴, —S(O)NR⁴⁴R^{44′}, —S(O)₂NR⁴⁴R^{44′}, —OS(O)NR⁴⁴R^{44′}, —OS(O)₂NR⁴⁴R^{44′}, —NR⁴⁴R^{44′}, —NR⁴⁴C(O)R⁴⁵, —NR⁴⁴C(O)OR⁴⁵, —NR⁴⁴C(O)NR⁴⁵R^{45′}, —NR⁴⁴S(O)R⁴⁵, —NR⁴⁴S(O)₂R⁴⁵, —NR⁴⁴S(O)NR⁴⁵R^{45′}, —NR⁴⁴S(O)₂NR⁴⁵R^{45′}, —C(O)R⁴⁴, —C(O)OR⁴⁴ or —C(O)NR⁴⁴R^{44′};

each R⁴¹ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁴⁶, —OC(O)R⁴⁶, —OC(O)NR⁴⁶R^{46′}, —OS(O)R⁴⁶, —OS(O)₂R⁴⁶, —SR⁴⁶, —S(O)R⁴⁶, —S(O)₂R⁴⁶, —S(O)NR⁴⁶R^{46′}, —S(O)₂NR⁴⁶R^{46′}, —OS(O)NR⁴⁶R^{46′}, —OS(O)₂NR⁴⁶R^{46′}, —NR⁴⁶R^{46′}, —NR⁴⁶C(O)R⁴⁷, —NR⁴⁶C(O)OR⁴⁷, —NR⁴⁶C(O)NR⁴⁷R^{47′}, —NR⁴⁶S(O)R⁴⁷, —NR⁴⁶S(O)₂R⁴⁷, —NR⁴⁶S(O)NR⁴⁷R^{47′}, —NR⁴⁶S(O)₂NR⁴⁷R^{47′}, —C(O)R⁴⁶, —C(O)OR⁴⁶ or —C(O)NR⁴⁶R^{46′};

each R⁴² is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR⁴³, —OC(O)R⁴³, —OC(O)NR⁴³R^{43′}, —OS(O)R⁴³, —OS(O)₂R⁴³, —SR⁴³, —S(O)R⁴³, —S(O)₂R⁴³, —S(O)NR⁴³R^{43′}, —S(O)₂NR⁴³R^{43′}, —OS(O)NR⁴³R^{43′}, —OS(O)₂NR⁴³R^{43′}, —NR⁴³R^{43′}, —C(O)R⁴³, —C(O)OR⁴³ or —C(O)NR⁴³R^{43′};

each R⁴³, R^{43′}, R⁴⁴, R^{44′}, R⁴⁵, R^{45′}, R⁴⁶, R^{46′}, R⁴⁷ and R^{47′} is independently selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl;

each R⁵², R^{52′}, R⁵³ and R^{53′} is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁵⁵, —OC(O)R⁵⁵, —OC(O)NR⁵⁵R^{55′}, —OS(O)R⁵⁵, —OS(O)₂R⁵⁵, —SR⁵⁵, —S(O)R⁵⁵, —S(O)₂R⁵⁵, —S(O)NR⁵⁵R^{55′}, —S(O)₂NR⁵⁵R^{55′}, —OS(O)NR⁵⁵R^{55′}, —OS(O)₂NR⁵⁵R^{55′}, —NR⁵⁵R^{55′}, —NR⁵⁵C(O)R⁵⁶, —NR⁵⁵C(O)OR⁵⁶, —NR⁵⁵C(O)NR⁵⁶R^{56′}, —NR⁵⁵S(O)R⁵⁶, —NR⁵⁵S(O)₂R⁵⁶, —NR⁵⁵S(O)NR⁵⁶R^{56′}, —NR⁵⁵S(O)₂NR⁵⁶R^{56′}, —C(O)R⁵⁵, —C(O)OR⁵⁵ or —C(O)NR⁵⁵R^{55′};

each R⁵⁴ and R^{54′} is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁵⁷, —OC(O)R⁵⁷, —OC(O)NR⁵⁷R^{57′}, —OS(O)R⁵⁷, —OS(O)₂R⁵⁷, —SR⁵⁷, —S(O)R⁵⁷, —S(O)₂R⁵⁷, —S(O)NR⁵⁷R^{57′}, —S(O)₂NR⁵⁷R^{57′}, —OS(O)NR⁵⁷R^{57′}, —OS(O)₂NR⁵⁷R^{57′}, —NR⁵⁷R^{57′}, —NR⁵⁷C(O)R⁵⁸, —NR⁵⁷C(O)OR⁵⁸, —NR⁵⁷C(O)NR⁵⁸R^{58′}, —NR⁵⁷S(O)R⁵⁸, —NR⁵⁷S(O)₂R⁵⁸, —NR⁵⁷S(O)NR⁵⁸R^{58′}, —NR⁵⁷S(O)₂NR⁵⁸R^{58′}, —C(O)R⁵⁷, —C(O)OR⁵⁷ or —C(O)NR⁵⁷R^{57′};

R⁵⁵, R^{55′}, R⁵⁶, R^{56′} R⁵⁷, R^{57′}, R⁵⁸ and R^{58′} are each independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl;

u is 1, 2, 3 or 4;

w is 1, 2, 3 or 4; and

w1 is 1, 2, 3 or 4;

or a pharmaceutically acceptable salt thereof.

31. The conjugate of any one of clauses 1 to 30, wherein each L¹ is selected from the group consisting of

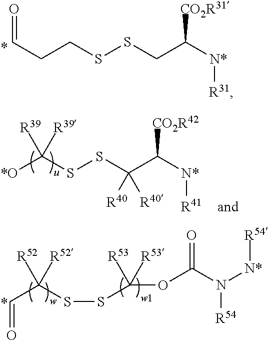

wherein

each R³¹ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};

each R^{31′} is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR^{32a}, —OC(O)R^{32a}, —OC(O)NR^{32a}R^{32a′}, —OS(O)R^{32a}, —OS(O)₂R^{32a}, —SR^{32a}, —S(O)R^{32a}, —S(O)₂R^{32a}, —S(O)NR^{32a}R^{32a′}, —S(O)₂NR^{32a}R^{32a′}, —OS(O)NR^{32a}R^{32a′}, —OS(O)₂NR^{32a}R^{32a′}, —NR^{32a}R^{32a′}, —C(O)R^{32a}, —C(O)OR³²a or —C(O)NR^{32a}R³²a′;

each R^{32a}, R^{32a′}, R³², R^{32′}, R³³ and R^{33′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl;

each R³⁹, R^{39′}, R⁴⁰ and R^{40′} is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁴⁴, —OC(O)R⁴⁴, —OC(O)NR⁴⁴R^{44′}, —OS(O)R⁴⁴, —OS(O)₂R⁴⁴, —SR′, —S(O)R⁴⁴, —S(O)₂R⁴⁴, —S(O)NR⁴⁴R^{44′}, —S(O)₂NR⁴⁴R^{44′}, —OS(O)NR⁴⁴R^{44′}, —OS(O)₂NR⁴⁴R^{44′}, —NR⁴⁴R^{44′}, —NR⁴⁴C(O)R⁴⁵, —NR⁴⁴C(O)OR⁴⁵, —NR⁴⁴C(O)NR⁴⁵R^{45′}, —NR⁴⁴S(O)R⁴⁵, —NR⁴⁴S(O)₂R⁴⁵, —NR⁴⁴S(O)NR⁴⁵R^{45′}, —NR⁴⁴S(O)₂NR⁴⁵R^{45′}, —C(O)R⁴⁴, —C(O)OR⁴⁴ or —C(O)NR⁴⁴R^{44′};

each R⁴¹ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁴⁶, —OC(O)R⁴⁶, —OC(O)NR⁴⁶R^{46′}, —OS(O)R⁴⁶, —OS(O)₂R⁴⁶, —SR⁴⁶, —S(O)R⁴⁶, —S(O)₂R⁴⁶, —S(O)NR⁴⁶R^{46′}, —S(O)₂NR⁴⁶R^{46′}, —OS(O)NR⁴⁶R^{46′}, —OS(O)₂NR⁴⁶R^{46′}, —NR⁴⁶R^{46′}, —NR⁴⁶C(O)R⁴⁷, —NR⁴⁶C(O)OR⁴⁷, —NR⁴⁶C(O)NR⁴⁷R^{47′}, —NR⁴⁶S(O)R⁴⁷, —NR⁴⁶S(O)₂R⁴⁷, —NR⁴⁶S(O)NR⁴⁷R^{47′}, —NR⁴⁶S(O)₂NR⁴⁷R^{47′}, —C(O)R⁴⁶, —C(O)OR⁴⁶ or —C(O)NR⁴⁶R^{46′};

each R⁴² is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR⁴³, —OC(O)R⁴³, —OC(O)NR⁴³R^{43′}, —OS(O)R⁴³, —OS(O)₂R⁴³, —SR⁴³, —S(O)R⁴³, —S(O)₂R⁴³, —S(O)NR⁴³R^{43′}, —S(O)₂NR⁴³R^{43′}, —OS(O)NR⁴³R^{43′}, —OS(O)₂NR⁴³R^{43′}, —NR⁴³R^{43′}, —C(O)R⁴³, —C(O)OR⁴³ or —C(O)NR⁴³R^{43′};

each R⁴³, R^{43′}, R⁴⁴, R^{44′}, R⁴⁵, R^{45′}, R⁴⁶, R^{46′}, R⁴⁷ and R^{47′} is independently selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl;

each R⁵², R^{52′}, R⁵³ and R^{53′} is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁵⁵, —OC(O)R⁵⁵, —OC(O)NR⁵⁵R^{55′}, —OS(O)R⁵⁵, —OS(O)₂R⁵⁵, —SR⁵⁵, —S(O)R⁵⁵, —S(O)₂R⁵⁵, —S(O)NR⁵⁵R^{55′}, —S(O)₂NR⁵⁵R^{55′}, —OS(O)NR⁵⁵R^{55′}, —OS(O)₂NR⁵⁵R^{55′}, —NR⁵⁵R^{55′}, —NR⁵⁵C(O)R⁵⁶, —NR⁵⁵C(O)OR⁵⁶, —NR⁵⁵C(O)NR⁵⁶R^{56′}, —NR⁵⁵S(O)R⁵⁶, —NR⁵⁵S(O)₂R⁵⁶, —NR⁵⁵S(O)NR⁵⁶R^{56′}, —NR⁵⁵S(O)₂NR⁵⁶R^{56′}, —C(O)R⁵⁵, —C(O)OR⁵⁵ or —C(O)NR⁵⁵R^{55′};

each R⁵⁴ and R^{54′} is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁵⁷, —OC(O)R⁵⁷, —OC(O)NR⁵⁷R^{57′}, —OS(O)R⁵⁷, —OS(O)₂R⁵⁷, —SR⁵⁷, —S(O)R⁵⁷, —S(O)₂R⁵⁷, —S(O)NR⁵⁷R^{57′}, —S(O)₂NR⁵⁷R^{57′}, —OS(O)NR⁵⁷R^{57′}, —OS(O)₂NR⁵⁷R^{57′}, —NR⁵⁷R^{57′}, —NR⁵⁷C(O)R⁵⁸, —NR⁵⁷C(O)OR⁵⁸, —NR⁵⁷C(O)NR⁵⁸R^{58′}, —NR⁵⁷S(O)R⁵⁸, —NR⁵⁷S(O)₂R⁵⁸, —NR⁵⁷S(O)NR⁵⁸R^{58′}, —NR⁵⁷S(O)₂NR⁵⁸R^{58′}, —C(O)R⁵⁷, —C(O)OR⁵⁷ or —C(O)NR⁵⁷R^{57′};

R⁵⁵, R^{55′}, R⁵⁶, R^{56′} R⁵⁷, R^{57′}, R⁵⁸ and R^{58′} are each independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl;

u is 1, 2, 3 or 4;

w is 1, 2, 3 or 4; and

w1 is 1, 2, 3 or 4;

or a pharmaceutically acceptable salt thereof.

32. The conjugate of any one of clauses 1 to 31, wherein each L² is selected from the group consisting of

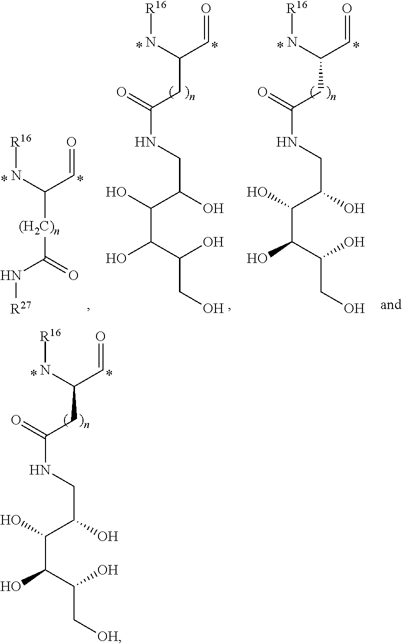

or a pharmaceutically acceptable salt thereof.

33. The conjugate of any one of clauses 1 to 32, wherein R¹⁶ is H, or a pharmaceutically acceptable salt thereof.

34. The conjugate of any one of clauses 1 to 32, or a pharmaceutically acceptable salt thereof, wherein D¹ is a drug selected from the group consisting of a cryptophycin, bortezomib, thiobortezomib, a tubulysin, aminopterin, rapamycin, paclitaxel, docetaxel, doxorubicin, daunorubicin, everolimus, α-amanatin, verucarin, didemnin B, geldanomycin, purvalanol A, ispinesib, budesonide, dasatinib, an epothilone, a maytansine, and a tyrosine kinase inhibitor.

35. The conjugate of any one of clauses 1 to 34, or a pharmaceutically acceptable salt thereof, wherein D¹ is a tubulysin.

36. The conjugate of any one of clauses 1 to 35, or a pharmaceutically acceptable salt thereof, wherein D¹ is a tetrapeptide of the formula III

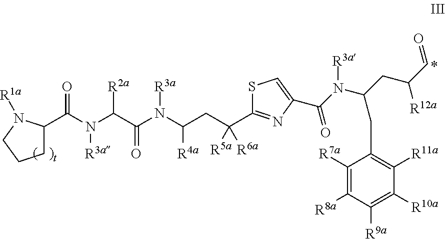

wherein

R^{1a}, R^{3a}, R^{3a′} and R^{3a″} are each independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR^{13a}, —OC(O)R^{13a}, —OC(O)NR^{13a}R^{13a′}, —OS(O)R^{13a}, —OS(O)₂R^{13a}, —SR^{13a}, —SC(O)R^{13a}, —S(O)R^{13a}, —S(O)₂R^{13a}, —S(O)₂OR^{13a}, —S(O)NR^{13a}R^{13a′}, —S(O)₂NR^{13a}R^{13a′}, —OS(O)NR^{13a}R^{13a′}, —OS(O)₂NR^{13a}R^{13a′}, —NR^{13a}R^{13a′}, —NR^{13a}C(O)R^{14a}, —NR^{13a}C(O)OR^{14a}, —NR^{13a}C(O)NR^{14a}R^{14a′}, —NR^{13a}S(O)R^{14a}, —NR^{13a}S(O)₂R^{14a}, —NR^{13a}S(O)NR^{13a}R^{13a′}, —NR^{13a}S(O)₂NR^{14a}R^{14a′}, —P(O)(OR^{13a})₂, —C(O)R^{13a}, —C(O)OR^{13a} or —C(O)NR^{13a}R^{13a′};

R^{2a}, R^{4a} and R^{12a} are each independently selected from the group consisting of H, D, C₁-C₆ alkyl, C₂-C₆ alkenyl, C₂-C₆ alkynyl;

R^{5a} and R^{6a} are each independently selected from the group consisting of H, D, halogen, C₁-C₆ alkyl, C₂-C₆ alkenyl, C₂-C₆ alkynyl, —OR^{15a}, —SR^{15a}, —OC(O)R^{15a}, —OC(O)NR^{15a}R^{15a′}, and —NR^{15a}R^{15a′}, wherein each hydrogen atom in C₁-C₆ alkyl, C₂-C₆ alkenyl and C₂-C₆ alkynyl is independently optionally substituted by halogen, —OR^{16a}, —SR¹⁶a, —NR^{16a}R^{16a′}, C(O)R^{16a}, —C(O)OR^{16a} or —C(O)NR^{16a}R^{16a′}; or R^{5a} and R^{6a} taken together with the carbon atom to which they are attached form a —C(O)—;

each R^{7a}, R^{8a}, R^{9a}, R^{10a} and R^{11a} is independently selected from the group consisting of H, D, halogen, C₁-C₆ alkyl, C₂-C₆ alkenyl, C₂-C₆ alkynyl, —CN, —NO₂, —NCO, —OR^{17a}, —SR^{17a}, —S(O)₂OR^{17a}, —NR^{17a}R^{17a′}, —P(O)(OR^{17a})₂, —C(O)R^{17a}, —C(O)OR^{17a} and —C(O)NR^{17a}R^{17a′}, wherein each hydrogen atom in C₁-C₆ alkyl, C₂-C₆ alkenyl and C₂-C₆ alkynyl is independently optionally substituted by halogen, —OR^{18a}, —SR^{18a}, —NR^{18a}R^{18a′}, —C(O)R^{18a}, —, —C(O)OR^{18a} or —C(O)NR^{18a}R^{18a′};

each R^{13a}, R^{13a′}, R^{14a}, R^{14a′}, R^{15a}, R^{15a′}, R^{16a}, R^{16a′}, R^{17a} and R^{17a′} is independently selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;

each R^{18a} and R^{18a′} is independently selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl —C(O)R^{19a}, —P(O)(OR^{19a})₂, and —S(O)₂OR^{19a},

each R¹⁹ is independently selected from H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl; and

t is 1, 2 or 3.

37. The conjugate of clause 36, or a pharmaceutically acceptable salt thereof, wherein t is 2.

38. The conjugate of clause 36 or 37, or a pharmaceutically acceptable salt thereof, wherein R^{1a} is C₁-C₆ alkyl.

39. The conjugate of any one of clauses 36 to 38, or a pharmaceutically acceptable salt thereof, wherein R^{1a} is methyl.

40. The conjugate of any one of clauses 36 to 39, or a pharmaceutically acceptable salt thereof, wherein R^{2a} is C₁-C₆ alkyl.

41. The conjugate of any one of clauses 36 to 40, or a pharmaceutically acceptable salt thereof, wherein R^{2a} is sec-butyl.

42. The conjugate of any one of clauses 36 to 41, or a pharmaceutically acceptable salt thereof, wherein R^{1a} is C₁-C₆ alkyl, wherein each hydrogen atom in C₁-C₆ alkyl is independently optionally substituted by —OC(O)R^{13a} and wherein R^{13a} is C₁-C₆ alkyl.

43. The conjugate of any one of clauses 36 to 42, or a pharmaceutically acceptable salt thereof, wherein R^{4a} is C₁-C₆ alkyl.

44. The conjugate of any one of clauses 36 to 43, or a pharmaceutically acceptable salt thereof, wherein R^{4a} is iso-propyl.

45. The conjugate of any one of clauses 36 to 44, or a pharmaceutically acceptable salt thereof, wherein R^{5a} is —OC(O)R^{15a}.

46. The conjugate of clause 45, or a pharmaceutically acceptable salt thereof, wherein R^{15a} is methyl.

47. The conjugate of any one of clauses 36 to 46, or a pharmaceutically acceptable salt thereof, wherein R^{6a} is H.

48. The conjugate of any one of clauses 36 to 47, or a pharmaceutically acceptable salt thereof, wherein R^{7a}, R^{8a}, R^{10a} and R^{11a} are H.

49. The conjugate of any one of clauses 36 to 48, or a pharmaceutically acceptable salt thereof, wherein R^{7a} is —OH.

50. The conjugate of any one of clauses 36 to 49, or a pharmaceutically acceptable salt thereof, wherein R^{12a} is C₁-C₆ alkyl.

51. The conjugate of any one of clauses 36 to 50, or a pharmaceutically acceptable salt thereof, wherein R^{12a} is methyl.

52. The conjugate of any one of clauses 36 to 51, or a pharmaceutically acceptable salt thereof, wherein R^{3a′} and R^{3a″} are H.

53. The conjugate of any one of clauses 36 to 52, or a pharmaceutically acceptable salt thereof, wherein D¹ is a tetrapeptide of the formula

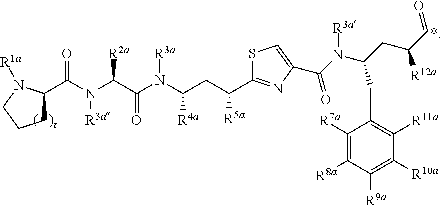

54. The conjugate of any one of clauses 36 to 53, or a pharmaceutically acceptable salt thereof, wherein D¹ is a tetrapeptide of the formula

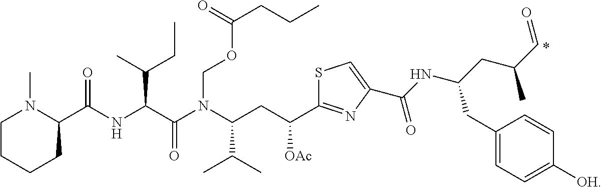 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C=O)cs1)C(C)C]

55. The conjugate of any one of clauses 1 to 54, or a pharmaceutically acceptable salt thereof, wherein L is of the formula

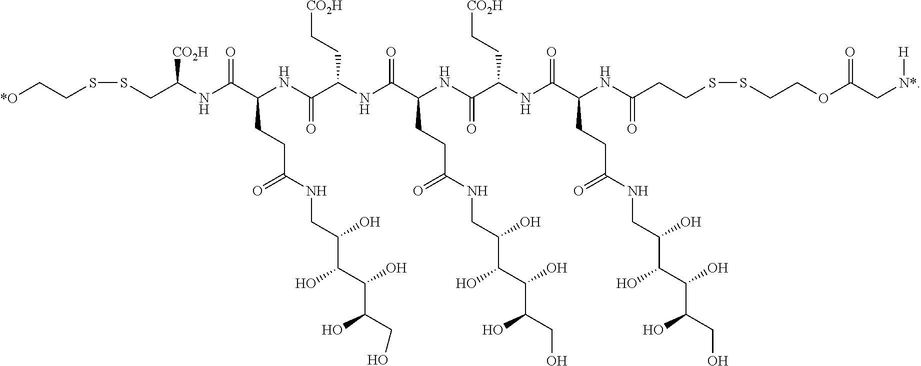

56. The conjugate of any one of clauses 1 to 54, or a pharmaceutically acceptable salt thereof, wherein L is of the formula

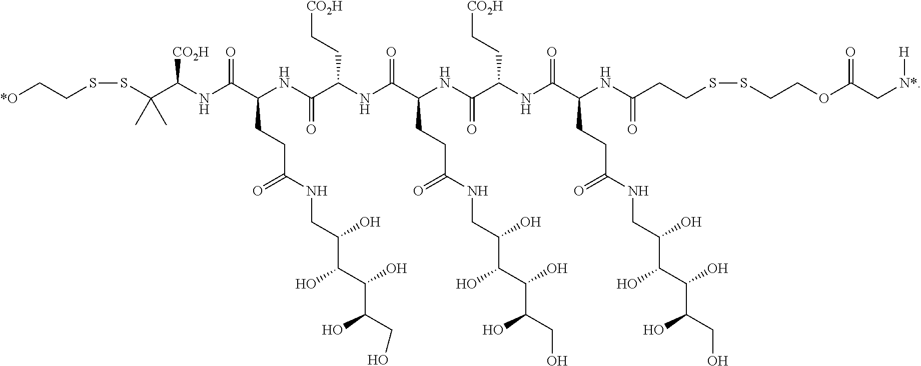

58. A pharmaceutical composition comprising a conjugate of any one of clauses 1 to 56, or a pharmaceutically acceptable salt thereof, and at least one excipient.

59. A method of treating abnormal cell growth in a mammal, including a human, the method comprising administering to the mammal a conjugate of any one of clauses 1-56.

60. The method of clause 59, wherein the abnormal cell growth is cancer

61. The method of clause 60, wherein the cancer is lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In another embodiment of said method, said abnormal cell growth is a benign proliferative disease, including, but not limited to, psoriasis, benign prostatic hypertrophy or restinosis.

62. Use of a conjugate according to any one of clauses 1 to 56 in the preparation of a medicament for the treatment of cancer.

63. Use of a conjugate according to any one of clauses 1 to 56 for the treatment of cancer.

### BRIEF DESCRIPTION OF THE DRAWINGS

FIG. 1 shows that single dose administration of the conjugates described herein are efficacious in vivo, as compared to an untreated control in mice having subcutaneous KB tumors. (▪) PBS treated control; (▾) EC2133 at 2 μmol/kg, single-dose {0,4,1}; all treatment groups were n=5; and each treatment group indicates {PR, CR, cure}.

FIG. 2 shows that single dose administration of the conjugates described herein are efficacious in vivo, as compared to an untreated control in mice having subcutaneous KB tumors. (▪) PBS treated control; (▾) EC2133 at 2 μmol/kg, SIW×2 {0,1,4}; all treatment groups were n=5; and each treatment group indicates {PR, CR, cure}.

### DEFINITIONS

As used herein, the term “alkyl” includes a chain of carbon atoms, which is optionally branched and contains from 1 to 20 carbon atoms. It is to be further understood that in certain embodiments, alkyl may be advantageously of limited length, including C₁-C₁₂, C₁-C₁₀, C₁-C₉, C₁-C₈, C₁-C₇, C₁-C₆, and C₁-C₄, Illustratively, such particularly limited length alkyl groups, including C₁-C₈, C₁-C₇, C₁-C₆, and C₁-C₄, and the like may be referred to as “lower alkyl.” Illustrative alkyl groups include, but are not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like. Alkyl may be substituted or unsubstituted. Typical substituent groups include cycloalkyl, aryl, heteroaryl, heteroalicyclic, hydroxy, alkoxy, aryloxy, mercapto, alkylthio, arylthio, cyano, halo, carbonyl, oxo, (═O), thiocarbonyl, O-carbamyl, N-carbamyl, O-thiocarbamyl, N-thiocarbamyl, C-amido, N-amido, C-carboxy, O-carboxy, nitro, and amino, or as described in the various embodiments provided herein. It will be understood that “alkyl” may be combined with other groups, such as those provided above, to form a functionalized alkyl. By way of example, the combination of an “alkyl” group, as described herein, with a “carboxy” group may be referred to as a “carboxyalkyl” group. Other non-limiting examples include hydroxyalkyl, aminoalkyl, and the like.

As used herein, the term “alkenyl” includes a chain of carbon atoms, which is optionally branched, and contains from 2 to 20 carbon atoms, and also includes at least one carbon-carbon double bond (i.e. C═C). It will be understood that in certain embodiments, alkenyl may be advantageously of limited length, including C₂-C₁₂, C₂-C₉, C₂-C₈, C₂-C₇, C₂-C₆, and C₂-C₄. Illustratively, such particularly limited length alkenyl groups, including C₂-C₈, C₂-C₇, C₂-C₆, and C₂-C₄ may be referred to as lower alkenyl. Alkenyl may be unsubstituted, or substituted as described for alkyl or as described in the various embodiments provided herein. Illustrative alkenyl groups include, but are not limited to, ethenyl, 1-propenyl, 2-propenyl, 1-, 2-, or 3-butenyl, and the like.

As used herein, the term “alkynyl” includes a chain of carbon atoms, which is optionally branched, and contains from 2 to 20 carbon atoms, and also includes at least one carbon-carbon triple bond (i.e. C≡C). It will be understood that in certain embodiments alkynyl may each be advantageously of limited length, including C₂-C₁₂, C₂-C₉, C₂-C₈, C₂-C₇, C₂-C₆, and C₂-C₄. Illustratively, such particularly limited length alkynyl groups, including C₂-C₈, C₂-C₇, C₂-C₆, and C₂-C₄ may be referred to as lower alkynyl. Alkenyl may be unsubstituted, or substituted as described for alkyl or as described in the various embodiments provided herein. Illustrative alkenyl groups include, but are not limited to, ethynyl, 1-propynyl, 2-propynyl, 1-, 2-, or 3-butynyl, and the like.

As used herein, the term “aryl” refers to an all-carbon monocyclic or fused-ring polycyclic groups of 6 to 12 carbon atoms having a completely conjugated pi-electron system. It will be understood that in certain embodiments, aryl may be advantageously of limited size such as C₆-C₁₀ aryl. Illustrative aryl groups include, but are not limited to, phenyl, naphthalenyl and anthracenyl. The aryl group may be unsubstituted, or substituted as described for alkyl or as described in the various embodiments provided herein.

As used herein, the term “cycloalkyl” refers to a 3 to 15 member all-carbon monocyclic ring, an all-carbon 5-member/6-member or 6-member/6-member fused bicyclic ring, or a multicyclic fused ring (a “fused” ring system means that each ring in the system shares an adjacent pair of carbon atoms with each other ring in the system) group where one or more of the rings may contain one or more double bonds but the cycloalkyl does not contain a completely conjugated pi-electron system. It will be understood that in certain embodiments, cycloalkyl may be advantageously of limited size such as C₃-C₁₃, C₃-C₆, C₃-C₆ and C₄-C₆. Cycloalkyl may be unsubstituted, or substituted as described for alkyl or as described in the various embodiments provided herein. Illustrative cycloalkyl groups include, but are not limited to, cyclopropyl, cyclobutyl, cyclopentyl, cyclopentenyl, cyclopentadienyl, cyclohexyl, cyclohexenyl, cycloheptyl, adamantyl, norbornyl, norbornenyl, 9H-fluoren-9-yl, and the like.

As used herein, the term “heterocycloalkyl” refers to a monocyclic or fused ring group having in the ring(s) from 3 to 12 ring atoms, in which at least one ring atom is a heteroatom, such as nitrogen, oxygen or sulfur, the remaining ring atoms being carbon atoms. Heterocycloalkyl may optionally contain 1, 2, 3 or 4 heteroatoms. Heterocycloalkyl may also have one of more double bonds, including double bonds to nitrogen (e.g. C═N or N═N) but does not contain a completely conjugated pi-electron system. It will be understood that in certain embodiments, heterocycloalkyl may be advantageously of limited size such as 3- to 7-membered heterocycloalkyl, 5- to 7-membered heterocycloalkyl, and the like. Heterocycloalkyl may be unsubstituted, or substituted as described for alkyl or as described in the various embodiments provided herein. Illustrative heterocycloalkyl groups include, but are not limited to, oxiranyl, thianaryl, azetidinyl, oxetanyl, tetrahydrofuranyl, pyrrolidinyl, tetrahydropyranyl, piperidinyl, 1,4-dioxanyl, morpholinyl, 1,4-dithianyl, piperazinyl, oxepanyl, 3,4-dihydro-2H-pyranyl, 5,6-dihydro-2H-pyranyl, 2H-pyranyl, 1, 2, 3, 4-tetrahydropyridinyl, and the like.

As used herein, the term “heteroaryl” refers to a monocyclic or fused ring group of 5 to 12 ring atoms containing one, two, three or four ring heteroatoms selected from nitrogen, oxygen and sulfur, the remaining ring atoms being carbon atoms, and also having a completely conjugated pi-electron system. It will be understood that in certain embodiments, heteroaryl may be advantageously of limited size such as 3- to 7-membered heteroaryl, 5- to 7-membered heteroaryl, and the like. Heteroaryl may be unsubstituted, or substituted as described for alkyl or as described in the various embodiments provided herein. Illustrative heteroaryl groups include, but are not limited to, pyrrolyl, furanyl, thiophenyl, imidazolyl, oxazolyl, thiazolyl, pyrazolyl, pyridinyl, pyrimidinyl, quinolinyl, isoquinolinyl, purinyl, tetrazolyl, triazinyl, pyrazinyl, tetrazinyl, quinazolinyl, quinoxalinyl, thienyl, isoxazolyl, isothiazolyl, oxadiazolyl, thiadiazolyl, triazolyl, benzimidazolyl, benzoxazolyl, benzthiazolyl, benzisoxazolyl, benzisothiazolyl and carbazoloyl, and the like.

As used herein, “hydroxy” or “hydroxyl” refers to an —OH group.

As used herein, “alkoxy” refers to both an —O-(alkyl) or an —O-(unsubstituted cycloalkyl) group. Representative examples include, but are not limited to, methoxy, ethoxy, propoxy, butoxy, cyclopropyloxy, cyclobutyloxy, cyclopentyloxy, cyclohexyloxy, and the like.

As used herein, “aryloxy” refers to an —O-aryl or an —O-heteroaryl group. Representative examples include, but are not limited to, phenoxy, pyridinyloxy, furanyloxy, thienyloxy, pyrimidinyloxy, pyrazinyloxy, and the like, and the like.

As used herein, “mercapto” refers to an —SH group.

As used herein, “alkylthio” refers to an —S-(alkyl) or an —S-(unsubstituted cycloalkyl) group. Representative examples include, but are not limited to, methylthio, ethylthio, propylthio, butylthio, cyclopropylthio, cyclobutylthio, cyclopentylthio, cyclohexylthio, and the like.

As used herein, “arylthio” refers to an —S-aryl or an —S-heteroaryl group. Representative examples include, but are not limited to, phenylthio, pyridinylthio, furanylthio, thienylthio, pyrimidinylthio, and the like.

As used herein, “halo” or “halogen” refers to fluorine, chlorine, bromine or iodine.

As used herein, “trihalomethyl” refers to a methyl group having three halo substituents, such as a trifluoromethyl group.

As used herein, “cyano” refers to a —CN group.

As used herein, “sulfinyl” refers to a —S(O)R″ group, where R″ is any R group as described in the various embodiments provided herein, or R″ may be a hydroxyl group.

As used herein, “sulfonyl” refers to a —S(O)₂R″ group, where R″ is any R group as described in the various embodiments provided herein, or R″ may be a hydroxyl group.

As used herein, “S-sulfonamido” refers to a —S(O)₂NR″R″ group, where R″ is any R group as described in the various embodiments provided herein.

As used herein, “N-sulfonamido” refers to a —NR″S(O)₂R″ group, where R″ is any R group as described in the various embodiments provided herein.

As used herein, “O-carbamyl” refers to a —OC(O)NR″R″ group, where R″ is any R group as described in the various embodiments provided herein.

As used herein, “N-carbamyl” refers to an R″OC(O)NR″— group, where R″ is any R group as described in the various embodiments provided herein.

As used herein, “O-thiocarbamyl” refers to a —OC(S)NR″R″ group, where R″ is any R group as described in the various embodiments provided herein.

As used herein, “N-thiocarbamyl” refers to a R″OC(S)NR″— group, where R″ is any R group as described in the various embodiments provided herein.

As used herein, “amino” refers to an —NR″R″ group, where R″ is any R group as described in the various embodiments provided herein.

As used herein, “C-amido” refers to a —C(O)NR″R″ group, where R″ is any R group as described in the various embodiments provided herein.

As used herein, “N-amido” refers to a R″C(O)NR″— group, where R″ is any R group as described in the various embodiments provided herein.

As used herein, “nitro” refers to a NO₂ group.

As used herein, “bond” refers to a covalent bond.

As used herein, “optional” or “optionally” means that the subsequently described event or circumstance may but need not occur, and that the description includes instances where the event or circumstance occurs and instances in which it does not. For example, “heterocycle group optionally substituted with an alkyl group” means that the alkyl may but need not be present, and the description includes situations where the heterocycle group is substituted with an alkyl group and situations where the heterocycle group is not substituted with the alkyl group.

As used herein, “independently” means that the subsequently described event or circumstance is to be read on its own relative to other similar events or circumstances. For example, in a circumstance where several equivalent hydrogen groups are optionally substituted by another group described in the circumstance, the use of “independently optionally” means that each instance of a hydrogen atom on the group may be substituted by another group, where the groups replacing each of the hydrogen atoms may be the same or different. Or for example, where multiple groups exist all of which can be selected from a set of possibilities, the use of “independently” means that each of the groups can be selected from the set of possibilities separate from any other group, and the groups selected in the circumstance may be the same or different.

As used herein, the term “pharmaceutically acceptable salt” refers to those salts which counter ions which may be used in pharmaceuticals. Such salts include:
(1) acid addition salts, which can be obtained by reaction of the free base of the parent conjugate with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or
(2) salts formed when an acidic proton present in the parent conjugate either is replaced by a metal ion, e.g., an alkali metal ion, an alkaline earth ion, or an aluminum ion; or coordinates with an organic base such as ethanolamine, diethanolamine, triethanolamine, trimethamine, N-methylglucamine, and the like.
   Pharmaceutically acceptable salts are well known to those skilled in the art, and any such pharmaceutically acceptable salt may be contemplated in connection with the embodiments described herein

As used herein, “amino acid” (a.k.a. “AA”) means any molecule that includes an alpha-carbon atom covalently bonded to an amino group and an acid group. The acid group may include a carboxyl group. “Amino acid” may include molecules having one of the formulas:

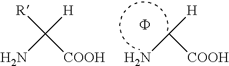

wherein R′ is a side group and Φ includes at least 3 carbon atoms. “Amino acid” includes stereoisomers such as the D-amino acid and L-amino acid forms. Illustrative amino acid groups include, but are not limited to, the twenty endogenous human amino acids and their derivatives, such as lysine (Lys), asparagine (Asn), threonine (Thr), serine (Ser), isoleucine (Ile), methionine (Met), proline (Pro), histidine (His), glutamine (Gln), arginine (Arg), glycine (Gly), aspartic acid (Asp), glutamic acid (Glu), alanine (Ala), valine (Val), phenylalanine (Phe), leucine (Leu), tyrosine (Tyr), cysteine (Cys), tryptophan (Trp), phosphoserine (PSER), sulfo-cysteine, arginosuccinic acid (ASA), hydroxyproline, phosphoethanolamine (PEA), sarcosine (SARC), taurine (TAU), carnosine (CARN), citrulline (CIT), anserine (ANS), 1,3-methyl-histidine (ME-HIS), alpha-amino-adipic acid (AAA), beta-alanine (BALA), ethanolamine (ETN), gamma-amino-butyric acid (GABA), beta-amino-isobutyric acid (BAIA), alpha-amino-butyric acid (BABA), L-allo-cystathionine (cystathionine-A; CYSTA-A), L-cystathionine (cystathionine-B; CYSTA-B), cystine, allo-isoleucine (ALLO-ILE), DL-hydroxylysine (hydroxylysine (I)), DL-allo-hydroxylysine (hydroxylysine (2)), ornithine (ORN), homocystine (HCY), and derivatives thereof. It will be appreciated that each of these examples are also contemplated in connection with the present disclosure in the D-configuration as noted above. Specifically, for example, D-lysine (D-Lys), D-asparagine (D-Asn), D-threonine (D-Thr), D-serine (D-Ser), D-isoleucine (D-Ile), D-methionine (D-Met), D-proline (D-Pro), D-histidine (D-His), D-glutamine (D-Gln), D-arginine (D-Arg), D-glycine (D-Gly), D-aspartic acid (D-Asp), D-glutamic acid (D-Glu), D-alanine (D-Ala), D-valine (D-Val), D-phenylalanine (D-Phe), D-leucine (D-Leu), D-tyrosine (D-Tyr), D-cysteine (D-Cys), D-tryptophan (D-Trp), D-citrulline (D-CIT), D-carnosine (D-CARN), and the like. In connection with the embodiments described herein, amino acids can be covalently attached to other portions of the conjugates described herein through their alpha-amino and carboxy functional groups (i.e. in a peptide bond configuration), or through their side chain functional groups (such as the side chain carboxy group in glutamic acid) and either their alpha-amino or carboxy functional groups. It will be understood that amino acids, when used in connection with the conjugates described herein, may exist as zwitterions in a conjugate in which they are incorporated.

As used herein, “sugar” refers to carbohydrates, such as monosaccharides, disaccharides, or oligosaccharides. In connection with the present disclosure, monosaccharides are preferred. Non-limiting examples of sugars include erythrose, threose, ribose, arabinose, xylose, lyxose, allose, altrose, glucose, mannose, galactose, ribulose, fructose, sorbose, tagatose, and the like. It will be undertsood that as used in connection with the present disclosure, sugar includes cyclic isomers of amino sugars, deoxy sugars, acidic sugars, and combinations thereof. Non-limiting examples of such sugars include, galactosamine, glucosamine, deoxyribose, fucose, rhamnose, glucuronic acid, ascorbic acid, and the like. In some embodiments, sugars for use in connection with the present disclosure include

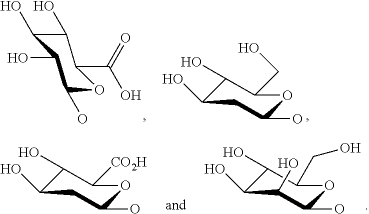

As used herein, “prodrug” refers to a compound that can be administered to a subject in a pharmacologically inactive form which then can be converted to a pharmacologically active form through a normal metabolic process, such as hydrolysis of an oxazolidine. It will be understood that the metabolic processes through which a prodrug can be converted to an active drug include, but are not limited to, one or more spontaneous chemical reaction(s), enzyme-catalyzed chemical reaction(s), and/or other metabolic chemical reaction(s), or a combination thereof. It will be appreciated that understood that a variety of metabolic processes are known in the art, and the metabolic processes through which the prodrugs described herein are converted to active drugs are non-limiting. A prodrug can be a precursor chemical compound of a drug that has a therapeutic effect on a subject.

As used herein, the term “therapeutically effective amount” refers to an amount of a drug or pharmaceutical agent that elicits the biological or medicinal response in a subject (i.e. a tissue system, animal or human) that is being sought by a researcher, veterinarian, medical doctor or other clinician, which includes, but is not limited to, alleviation of the symptoms of the disease or disorder being treated. In one aspect, the therapeutically effective amount is that amount of an active which may treat or alleviate the disease or symptoms of the disease at a reasonable benefit/risk ratio applicable to any medical treatment. In another aspect, the therapeutically effective amount is that amount of an inactive prodrug which when converted through normal metabolic processes to produce an amount of active drug capable of eliciting the biological or medicinal response in a subject that is being sought.

It is also appreciated that the dose, whether referring to monotherapy or combination therapy, is advantageously selected with reference to any toxicity, or other undesirable side effect, that might occur during administration of one or more of the conjugates described herein. Further, it is appreciated that the co-therapies described herein may allow for the administration of lower doses of conjugates that show such toxicity, or other undesirable side effect, where those lower doses are below thresholds of toxicity or lower in the therapeutic window than would otherwise be administered in the absence of a cotherapy.

As used herein, “administering” includes all means of introducing the conjugates and compositions described herein to the host animal, including, but are not limited to, oral (po), intravenous (iv), intramuscular (im), subcutaneous (sc), transdermal, inhalation, buccal, ocular, sublingual, vaginal, rectal, and the like. The conjugates and compositions described herein may be administered in unit dosage forms and/or formulations containing conventional nontoxic pharmaceutically-acceptable carriers, adjuvants, and/or vehicles.

As used herein “pharmaceutical composition” or “composition” refers to a mixture of one or more of the conjugates described herein, or pharmaceutically acceptable salts, solvates, hydrates thereof, with other chemical components, such as pharmaceutically acceptable excipients. The purpose of a pharmaceutical composition is to facilitate administration of a conjugate to a subject. Pharmaceutical compositions suitable for the delivery of conjugates described and methods for their preparation will be readily apparent to those skilled in the art. Such compositions and methods for their preparation may be found, for example, in ‘Remington's Pharmaceutical Sciences’, 19th Edition (Mack Publishing Company, 1995).

As used herein, a “pharmaceutically acceptable excipient” refers to an inert substance added to a pharmaceutical composition to further facilitate administration of a conjugate such as a diluent or a carrier.

### DETAILED DESCRIPTION

In each of the foregoing and each of the following embodiments, it is to be understood that the formulae include and represent not only all pharmaceutically acceptable salts of the conjugates, but also include any and all hydrates and/or solvates of the conjugate formulae. It is appreciated that certain functional groups, such as the hydroxy, amino, and like groups form complexes and/or coordination conjugates with water and/or various solvents, in the various physical forms of the conjugates. Accordingly, the above formulae are to be understood to include and represent those various hydrates and/or solvates. It is also to be understood that the non-hydrates and/or non-solvates of the conjugate formulae are described by such formula, as well as the hydrates and/or solvates of the conjugate formulae.

The conjugates described herein can be expressed by the generalized descriptors B, L and D¹, for example B-L-D¹, where B is a cell surface receptor binding ligand (a.k.a. a “binding ligand”), L is a linker that may include one or more releasable portions (i.e. a releasable linker) and L may be described by, for example, one or more of the groups AA, L¹ or L² as defined herein, and D¹ represents a drug covalently attached to the conjugates described herein.

The conjugates described herein can be described according to various embodiments including but not limited to

B-L¹-L²-AA-L²-AA-L²-L¹-D¹

wherein B, AA, L¹, L² and D² are defined by the various embodiments described herein, or a pharmaceutically acceptable salt thereof.

As used herein, the term cell surface receptor binding ligand (aka a “binding ligand”), generally refers to compounds that bind to and/or target receptors that are found on cell surfaces, and in particular those that are found on, over-expressed by, and/or preferentially expressed on the surface of pathogenic cells. Illustrative ligands include, but are not limited to, vitamins and vitamin receptor binding compounds.

Illustrative vitamin moieties include carnitine, inositol, lipoic acid, pyridoxal, ascorbic acid, niacin, pantothenic acid, folic acid, riboflavin, thiamine, biotin, vitamin B₁₂, and the lipid soluble vitamins A, D, E and K. These vitamins, and their receptor-binding analogs and derivatives, constitute the targeting entity covalently attachment to the linker. Illustrative biotin analogs that bind to biotin receptors include, but are not limited to, biocytin, biotin sulfoxide, oxybiotin, and the like).

Illustrative folic acid analogs that bind to folate receptors include, but are not limited to folinic acid, pteropolyglutamic acid, and folate receptor-binding pteridines such as tetrahydropterins, dihydrofolates, tetrahydrofolates, and their deaza and dideaza analogs. The terms “deaza” and “dideaza” analogs refer to the art-recognized analogs having a carbon atom substituted for one or two nitrogen atoms in the naturally occurring folic acid structure, or analog or derivative thereof. For example, the deaza analogs include the 1-deaza, 3-deaza, 5-deaza, 8-deaza, and 10-deaza analogs of folate, folinic acid, pteropolyglutamic acid, and folate receptor-binding pteridines such as tetrahydropterins, dihydrofolates, and tetrahydrofolates. The dideaza analogs include, for example, 1,5-dideaza, 5,10-dideaza, 8,10-dideaza, and 5,8-dideaza analogs of folate, folinic acid, pteropolyglutamic acid, and folate receptor-binding pteridines such as tetrahydropterins, dihydrofolates, and tetrahydrofolates. Other folates useful as complex forming ligands for this disclosure are the folate receptor-binding analogs aminopterin, amethopterin (also known as methotrexate), N¹⁰-methylfolate, 2-deamino-hydroxyfolate, deaza analogs such as 1-deazamethopterin or 3-deazamethopterin, and 3′,5′-dichloro-4-amino-4-deoxy-N¹⁰-methylpteroylglutamic acid (dichloromethotrexate). The foregoing folic acid analogs and/or derivatives are conventionally termed “folates,” reflecting their ability to bind with folate-receptors, and such ligands when conjugated with exogenous molecules are effective to enhance transmembrane transport, such as via folate-mediated endocytosis as described herein.

In some embodiments, B is of the formula I

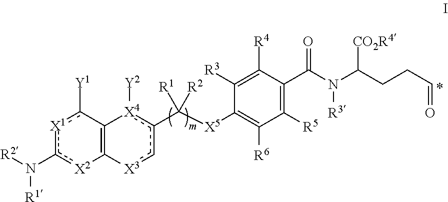

wherein

R¹ and R² in each instance are independently selected from the group consisting of H, D, halogen, C₁-C₆ alkyl, C₂-C₆ alkenyl, C₂-C₆ alkynyl, —OR⁷, —SR⁷ and —NR⁷R^{7′}, wherein each hydrogen atom in C₁-C₆ alkyl, C₂-C₆ alkenyl and C₂-C₆ alkynyl is independently optionally substituted by halogen, —OR⁸, —SR⁸, —NR⁸R^{8′}, —C(O)R⁸, —C(O)OR⁸ or —C(O)NR⁸R^{8′};

R³, R⁴, R⁵ and R⁶ are each independently selected from the group consisting of H, D, halogen, C₁-C₆ alkyl, C₂-C₆ alkenyl, C₂-C₆ alkynyl, —CN, —NO₂, —NCO, —OR⁹, —SR⁹, —NR⁹R^{9′}, —C(O)R⁹, —C(O)OR⁹ and —C(O)NR⁹R^{9′}, wherein each hydrogen atom in C₁-C₆ alkyl, C₂-C₆ alkenyl and C₂-C₆ alkynyl is independently optionally substituted by halogen, —OR¹⁰, —SR¹⁰, —NR¹⁰R^{10′}, —C(O)R¹⁰, —C(O)OR¹⁰ or —C(O)NR¹⁰R^{10′};

each R⁷, R^{7′}, R⁸, R^{8′}, R⁹, R^{9′}, R¹⁰ and R^{10′} is independently H, D, C₁-C₆ alkyl, C₂-C₆ alkenyl or C₂-C₆ alkynyl;

X¹ is NR¹¹—, ═N—, —N═, —C(R¹¹)═ or ═C(R¹¹)—;

X² is NR^{11′}- or ═N—;

X³ is NR^{11″}—, —N═ or —C(R^{11′})=;

X⁴ is N═ or C═;

X⁵ is NR¹² or CR¹²R^{12′};

Y¹ is H, D, —OR¹³, —SR¹³ or —NR¹³R^{13′} when X¹ is —N═ or —C(R¹¹)═, or Y¹ is ═O when X¹ is NR¹¹—, ═N— or ═C(R¹¹)—;

Y² is H, D, C₁-C₆ alkyl, C₂-C₆ alkenyl, —C(O)R¹⁴, —C(O)OR¹⁴ or —C(O)NR¹⁴R^{14′} when X⁴ is —C═, or Y² is absent when X⁴ is N═;

R^{1′}, R^{2′}, R^{3′}, R^{4′}, R¹¹, R^{11′}, R^{11″}, R¹², R^{12′}, R¹³, R^{13′}, R¹⁴ and R^{14′} are each independently selected from the group consisting of H, D, C₁-C₆ alkyl, C₂-C₆ alkenyl, C₂-C₆ alkynyl, —C(O)R¹⁵, —C(O)OR¹⁵ and —C(O)NR¹⁵R^{15′};

R¹⁵ and R^{15′} are each independently H or C₁-C₆ alkyl;

m is 1, 2, 3 or 4; and

* is a covalent bond.

L¹ is a releasable linker. As used herein, the term “releasable linker” refers to a linker that includes at least one bond that can be broken under physiological conditions, such as a pH-labile, acid-labile, base-labile, oxidatively labile, metabolically labile, biochemically labile, or enzyme-labile bond. It is appreciated that such physiological conditions resulting in bond breaking do not necessarily include a biological or metabolic process, and instead may include a standard chemical reaction, such as a hydrolysis reaction, for example, at physiological pH, or as a result of compartmentalization into a cellular organelle such as an endosome having a lower pH than cytosolic pH.

It is understood that a cleavable bond can connect two adjacent atoms within the releasable linker and/or connect other linkers, B or D¹, as described herein, at either or both ends of the releasable linker. In the case where a cleavable bond connects two adjacent atoms within the releasable linker, following breakage of the bond, the releasable linker is broken into two or more fragments. Alternatively, in the case where a cleavable bond is between the releasable linker and another moiety, such as another linker, a drug or binding ligand, the releasable linker becomes separated from the other moiety following breaking of the bond.

The lability of the cleavable bond can be adjusted by, for example, substituents at or near the cleavable bond, such as including alpha-branching adjacent to a cleavable disulfide bond, increasing the hydrophobicity of substituents on silicon in a moiety having silicon-oxygen bond that may be hydrolyzed, homologating alkoxy groups that form part of a ketal or acetal that may be hydrolyzed, and the like.

In some embodiments, releasable linkers described herein include one or more cleavable functional groups, such as a disulfide, a carbonate, a carbamate, an amide, an ester, and the like. Illustrative releasable linkers described herein include linkers that include hemiacetals and sulfur variations thereof, acetals and sulfur variations thereof, hemiaminals, aminals, and the like, and can be formed from methylene fragments substituted with at least one heteroatom, 1-alkoxy alkylene, 1-alkoxycycloalkylene, 1-alkoxyalkylenecarbonyl, 1-alkoxycycloalkylene-carbonyl, and the like. Illustrative releasable linkers described herein include linkers that include carbonylarylcarbonyl, carbonyl(carboxyaryl)carbonyl, carbonyl(biscarboxyaryl)carbonyl, haloalkylenecarbonyl, and the like. Illustrative releasable linkers described herein include linkers that include alkylene(dialkylsilyl), alkylene(alkylarylsilyl), alkylene(diarylsilyl), (dialkylsilyl)aryl, (alkylarylsilyl)aryl, (diarylsilyl)aryl, and the like. Illustrative releasable linkers described herein include oxycarbonyloxy, oxycarbonyloxyalkyl, sulfonyloxy, oxysulfonylalkyl, and the like. Illustrative releasable linkers described herein include linkers that include iminoalkylidenyl, carbonylalkylideniminyl, iminocycloalkylidenyl, carbonylcycloalkyliden-iminyl, and the like. Illustrative releasable linkers described herein include linkers that include alkylenethio, alkylenearylthio, and carbonylalkylthio, and the like.

In some embodiments, the conjugates described herein comprise more than one releasable linker. It will be appreciated that when the conjugates described herein comprise more than one releasable linker, the releasable linkers may be the same. It will be further appreciated that when the conjugates described herein comprise more than one releasable linker, the releasable linkers may be different. In some embodiments, the conjugates described herein comprise more than one releasable linker, wherein the more than one releasable linker comprises in each instance a disulfide bond. In some embodiments, the conjugates described herein comprise two releasable linkers both of which include a disulfide bond.

In some embodiments, each L¹ is independently selected from the group consisting of

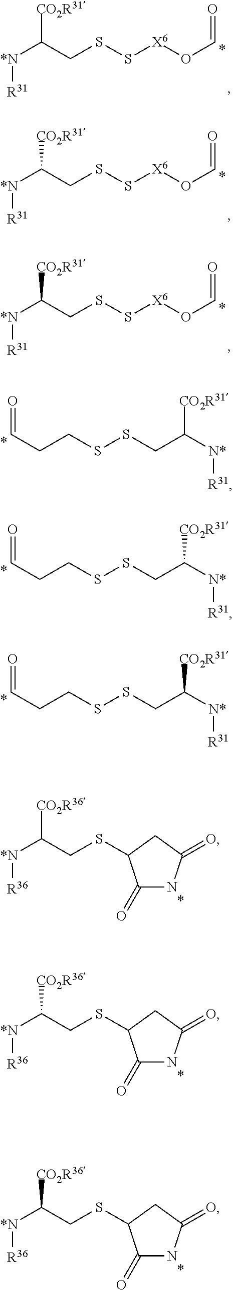

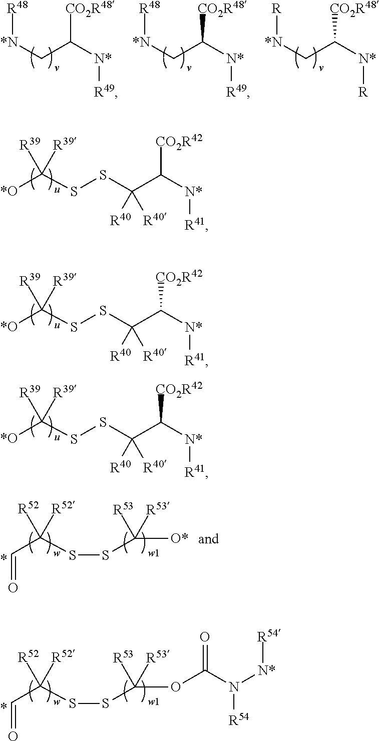

wherein

each R³¹ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};

each R^{31′} is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR^{32a}, —OC(O)R^{32a}, —OC(O)NR^{32a}R^{32a′}, —OS(O)R^{32a}, —OS(O)₂R^{32a}, —SR^{32a}, —S(O)R^{32a}, —S(O)₂R^{32a}, —S(O)NR^{32a}R^{32a′}, —S(O)₂NR^{32a}R^{32a′}, —OS(O)NR^{32a}R^{32a′}, —OS(O)₂NR^{32a}R^{32a′}, —NR^{32a}R^{32a′}, —C(O)R^{32a}, —C(O)OR^{32a} or —C(O)NR^{32a}R^{32a′};

each X⁶ is independently C₁-C₆ alkyl or C₆-C₁₀ aryl(C₁-C₆ alkyl), wherein each hydrogen atom in C₁-C₆ alkyl and C₆-C₁₀ aryl(C₁-C₆ alkyl) is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —OS(O)₂R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —OS(O)₂NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴, —C(O)OR³⁴ or —C(O)NR³⁴R^{34′};

each R^{32a}, R^{32a′}, R³², R^{32′}, R³³, R^{33′}, R³⁴, R^{34′}, R³⁵ and R^{35′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl;

each R³⁶ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁷, —OC(O)R³⁷, —OC(O)NR³⁷R^{37′}, —OS(O)R³⁷, —OS(O)₂R³⁷, —SR³⁷, —S(O)R³⁷, —S(O)₂R³⁷, —S(O)NR³⁷R^{37′}, —S(O)₂NR³⁷R^{37′}, —OS(O)NR³⁷R^{37′}, —OS(O)₂NR³⁷R^{37′}, —NR³⁷R^{37′}, —NR³⁷C(O)R³⁸, —NR³⁷C(O)OR³⁸, —NR³⁷C(O)NR³⁸R^{38′}, —NR³⁷S(O)R³⁸, —NR³⁷S(O)₂R³⁸, —NR³⁷S(O)NR³⁸R^{38′}, —NR³⁷S(O)₂NR³⁸R^{38′}, —C(O)R³⁷, —C(O)OR³⁷ or —C(O)NR³⁷R^{37′};

each R^{36′} is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR^{37a}, —OC(O)R^{37a}, —OC(O)NR^{37a}R^{37a′}, —OS(O)R^{37a}, —OS(O)₂R^{37a}, —SR^{37a}, —S(O)R^{37a}, —S(O)₂R^{37a}, —S(O)NR^{37a}R^{37a′}, —S(O)₂NR^{37a}R^{37a′}, —OS(O)NR^{37a}R^{37a′}, —OS(O)₂NR^{37a}R^{37a′}, —NR^{37a}R^{37a′}, —C(O)R^{37a}, —C(O)OR^{37a} or —C(O)NR^{37a}R^{37a′};

each R³⁷, R^{37′}, R^{37a}, R^{37a′}, R³⁸ and R^{38′ ′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl;

each R³⁹, R^{39′}, R⁴⁰ and R^{40′} is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁴⁴, —OC(O)R⁴⁴, —OC(O)NR⁴⁴R^{44′}, —OS(O)R⁴⁴, —OS(O)₂R⁴⁴, —SR⁴⁴, —S(O)R⁴⁴, —S(O)₂R⁴⁴, —S(O)NR⁴⁴R^{44′}, —S(O)₂NR⁴⁴R^{44′}, —OS(O)NR⁴⁴R^{44′}, —OS(O)₂NR⁴⁴R^{44′}, —NR⁴⁴R^{44′}, —NR⁴⁴C(O)R⁴⁵, —NR⁴⁴C(O)OR⁴⁵, —NR⁴⁴C(O)NR⁴⁵R^{45′}, —NR⁴⁴S(O)R⁴⁵, —NR⁴⁴S(O)₂R⁴⁵, —NR⁴⁴S(O)NR⁴⁵R^{45′}, —NR⁴⁴S(O)₂NR⁴⁵R^{45′}, —C(O)R⁴⁴, —C(O)OR⁴⁴ or —C(O)NR⁴⁴R^{44′};

each R⁴¹ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁴⁶, —OC(O)R⁴⁶, —OC(O)NR⁴⁶R^{46′}, —OS(O)R⁴⁶, —OS(O)₂R⁴⁶, —SR¹⁰, —S(O)R⁴⁶, —S(O)₂R⁴⁶, —S(O)NR⁴⁶R^{46′}, —S(O)₂NR⁴⁶R^{46′}, —OS(O)NR⁴⁶R^{46′}, —OS(O)₂NR⁴⁶R^{46′}, —NR⁴⁶R^{46a′}, —NR⁴⁶C(O)R⁴⁷, —NR⁴⁶C(O)OR⁴⁷, —NR⁴⁶C(O)NR⁴⁷R^{47′}, —NR⁴⁶S(O)R⁴⁷, —NR⁴⁶S(O)₂R⁴⁷, —NR⁴⁶S(O)NR⁴⁷R^{47′}, —NR⁴⁶S(O)₂NR⁴⁷R^{47′}, —C(O)R⁴⁶, —C(O)OR⁴⁶ or —C(O)NR⁴⁶R^{46′};

each R⁴² is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR⁴³, —OC(O)R⁴³, —OC(O)NR⁴³R^{43′}, —OS(O)R⁴³, —OS(O)₂R⁴³, —SR⁴³, —S(O)R⁴³, —S(O)₂R⁴³, —S(O)NR⁴³R^{43′}, —S(O)₂NR⁴³R^{43′}, —OS(O)NR⁴³R^{43′}, —OS(O)₂NR⁴³R^{43′}, —NR⁴³R^{43′}, —C(O)R⁴³, —C(O)OR⁴³ or —C(O)NR⁴³R^{43′};

each R⁴³, R^{43′}, R⁴⁴, R^{44′}, R⁴⁵, R^{45′}, R⁴⁶, R^{46′}, R⁴⁷ and R^{47′} is independently selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl;

each R⁴⁸ and R⁴⁹ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁵⁰, —OC(O)R⁵⁰, —OC(O)NR⁵⁰R^{50′}, —OS(O)R⁵⁰, —OS(O)₂R⁵⁰, —SR⁵⁰, —S(O)R⁵⁰, —S(O)₂R⁵⁰, —S(O)NR⁵⁰R^{50′}, —S(O)₂NR⁵⁰R^{50′}, —OS(O)NR⁵⁰R^{50′}, —OS(O)₂NR⁵⁰R^{50′}, —NR⁵⁰R^{50′}, —NR⁵⁰C(O)R⁵¹, —NR⁵⁰C(O)OR⁵¹, —NR⁵⁰C(O)NR⁵¹R^{51′}, —NR⁵⁰S(O)R⁵¹, —NR⁵⁰S(O)₂R⁵¹, —NR⁵⁰S(O)NR⁵¹R^{51′}, —NR⁵⁰S(O)₂NR⁵¹R^{51′}, —C(O)R⁵⁰, —C(O)OR⁵⁰ or —C(O)NR⁵⁰R^{50′};

each R⁴⁸ is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR^{48a}, —OC(O)R^{48a}, —OC(O)NR^{48a}R^{48a′}, —OS(O)R^{48a}, —OS(O)₂R^{48a}, —SR^{48a}, —S(O)R^{48a}, —S(O)₂R^{48a}, —S(O)NR^{48a}R^{48a′}, S(O)₂NR^{48a}R^{48a′}, —OS(O)NR^{48a}R^{48a′}, —OS(O)₂NR^{48a}R^{48a′}, —NR^{48a}R^{48a′}, —C(O)R^{48a}, —C(O)OR^{48a} or —C(O)NR^{48a}R^{48a′};

each R^{48a}, R^{48a′}, R⁵⁰, R^{50′}, R⁵¹ and R^{51′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl;

each R⁵², R^{52′}, R⁵³ and R^{53′} is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁵⁵, —OC(O)R⁵⁵, —OC(O)NR⁵⁵R^{55′}, —OS(O)R⁵⁵, —OS(O)₂R⁵⁵, —SR⁵⁵, —S(O)R⁵⁵, —S(O)₂R⁵⁵, —S(O)NR⁵⁵R^{55′}, —S(O)₂NR⁵⁵R^{55′}, —OS(O)NR⁵⁵R^{55′}, —OS(O)₂NR⁵⁵R^{55′}, —NR⁵⁵R^{55′}, —NR⁵⁵C(O)R⁵⁶, —NR⁵⁵C(O)OR⁵⁶, —NR⁵⁵C(O)NR⁵⁶R^{56′}, —NR⁵⁵S(O)R⁵⁶, —NR⁵⁵S(O)₂R⁵⁶, —NR⁵⁵S(O)NR⁵⁶R^{56′}, —NR⁵⁵S(O)₂NR⁵⁶R^{56′}, —C(O)R⁵⁵, —C(O)OR⁵⁵ or —C(O)NR⁵⁵R^{55′};

each R⁵⁴ and R^{54′} is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁵⁷, —OC(O)R⁵⁷, —OC(O)NR⁵⁷R^{57′}, —OS(O)R⁵⁷, —OS(O)₂R⁵⁷, —SR⁵⁷, —S(O)R⁵⁷, —S(O)₂R⁵⁷, —S(O)NR⁵⁷R^{57′}, —S(O)₂NR⁵⁷R^{57′}, —OS(O)NR⁵⁷R^{57′}, —OS(O)₂NR⁵⁷R^{57′}, —NR⁵⁷R^{57′}, —NR⁵⁷C(O)R⁵⁸, —NR⁵⁷C(O)OR⁵⁸, —NR⁵⁷C(O)NR⁵⁸R^{58′}, —NR⁵⁷S(O)R⁵⁸, —NR⁵⁷S(O)₂R⁵⁸, —NR⁵⁷S(O)NR⁵⁸R^{58′}, —NR⁵⁷S(O)₂NR⁵⁸R^{58′}, —C(O)R⁵⁷, —C(O)OR⁵⁷ or —C(O)NR⁵⁷R^{57′};

R⁵⁵, R^{55′}, R⁵⁶, R^{56′} R⁵⁷, R^{57′}, R⁵⁸ and R^{58′} are each independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl;

u is 1, 2, 3 or 4;

v is 1, 2, 3, 4, 5 or 6;

w is 1, 2, 3 or 4;

w1 is 1, 2, 3 or 4; and

* is a covalent bond.

In some embodiments, R³¹ is H. In some embodiments, R³⁶ is H. In some embodiments, X⁶ is C₁-C₆ alkyl. In some embodiments, X⁶ is C₁-C₆ alkyl. C₆-C₁₀ aryl(C₁-C₆ alkyl). In some embodiments, one or more L¹ is of the formula

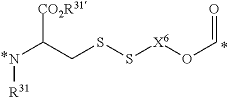

wherein

each R³¹ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};

each R^{31′} is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR^{32a}, —OC(O)R^{32a}, —OC(O)NR^{32a}R^{32a′}, —OS(O)R^{32a}, —OS(O)₂R^{32a}, —SR^{32a}, —S(O)R^{32a}, —S(O)₂R^{32a}, —S(O)NR^{32a}R^{32a′}, —S(O)₂NR^{32a}R^{32a′} —OS(O)NR^{32a}R^{32a′}, —OS(O)₂NR^{32a}R^{32a′} —NR^{32a}R^{32a′} —C(O)R^{32a}, —C(O)OR^{32a} or —C(O)NR^{32a}R^{32a′};

each X⁶ is independently C₁-C₆ alkyl or C₆-C₁₀ aryl(C₁-C₆ alkyl), wherein each hydrogen atom in C₁-C₆ alkyl and C₆-C₁₀ aryl(C₁-C₆ alkyl) is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —OS(O)₂R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —OS(O)₂NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴, —C(O)OR³⁴ or —C(O)NR³⁴R^{34′};

each R^{32a}, R^{32a′}, R³², R^{32′}, R³³, R^{33′}, R³⁴, R^{34′}, R³⁵ and R^{35′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl; and

* is a covalent bond.

In some embodiments, R³¹ is H, and X⁶ is C₁-C₆ alkyl. In some embodiments, R³¹ is H, and X⁶ is C₆-C₁₀ aryl(C₁-C₆ alkyl).

In some embodiments, one or more L¹ is of the formula

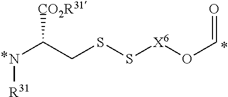

wherein

each R³¹ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};

each R^{31′} is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR^{32a}, —OC(O)R^{32a}, —OC(O)NR^{32a}R^{32a′} —OS(O)R^{32a}, —OS(O)₂R^{32a}, —SR^{32a}, —S(O)R^{32a}, —S(O)₂R^{32a}, —S(O)NR^{32a}R^{32a′}, —S(O)₂NR^{32a}R^{32a′} —OS(O)NR^{32a}R^{32a′}, —OS(O)₂NR^{32a}R^{32a′} —NR^{32a}R^{32a′} —C(O)R^{32a}, —C(O)OR^{32a} or —C(O)NR^{32a}R^{32a′};

each X⁶ is independently C₁-C₆ alkyl or C₆-C₁₀ aryl(C₁-C₆ alkyl), wherein each hydrogen atom in C₁-C₆ alkyl and C₆-C₁₀ aryl(C₁-C₆ alkyl) is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —OS(O)₂R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —OS(O)₂NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴, —C(O)OR³⁴ or —C(O)NR³⁴R^{34′};

each R^{32a}, R^{32a′}, R³², R^{32′}, R³³, R^{33′}, R³⁴, R^{34′}, R³⁵ and R^{35′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl; and

* is a covalent bond.

In some embodiments, R³¹ is H, and X⁶ is C₁-C₆ alkyl. In some embodiments, R³¹ is H, and X⁶ is C₆-C₁₀ aryl(C₁-C₆ alkyl).

In some embodiments, one or more L¹ is of the formula

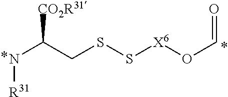

wherein

each R³¹ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};

each R^{31′} is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR^{32a}, —OC(O)R^{32a}, —OC(O)NR^{32a}R^{32a′}, —OS(O)R^{32a}, —OS(O)₂R^{32a}, —SR^{32a}, —S(O)R^{32a}, —S(O)₂R^{32a}, —S(O)NR^{32a}R^{32a′}, —S(O)₂NR^{32a}R^{32a′}, —OS(O)NR^{32a}R^{32a′}, —OS(O)₂NR^{32a}R^{32a′}, —NR^{32a}R^{32a′}, —C(O)R^{32a}, —C(O)OR^{32a} or —C(O)NR^{32a}R^{32a′};

each X⁶ is independently C₁-C₆ alkyl or C₆-C₁₀ aryl(C₁-C₆ alkyl), wherein each hydrogen atom in C₁-C₆ alkyl and C₆-C₁₀ aryl(C₁-C₆ alkyl) is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —OS(O)₂R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —OS(O)₂NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴, —C(O)OR³⁴ or —C(O)NR³⁴R^{34′};

each R^{32a}, R^{32a′}, R³², R^{32′}, R³³, R^{33′}, R³⁴, R^{34′}, R³⁵ and R^{35′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl; and

* is a covalent bond.

In some embodiments, R³¹ is H, and X⁶ is C₁-C₆ alkyl. In some embodiments, R³¹ is H, and X⁶ is C₆-C₁₀ aryl(C₁-C₆ alkyl).

In some embodiments, one or more L¹ is of the formula

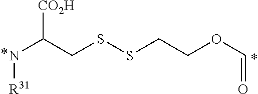

R³¹ is selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};

each R³², R^{32′}, R³³ and R^{33′} are independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl; and

* is a covalent bond. In some embodiments, R³¹ is H.

In some embodiments, one or more L¹ is of the formula

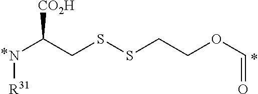

R³¹ is selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};

each R³², R^{32′}, R³³ and R^{33′} are independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl; and

* is a covalent bond. In some embodiments, R³¹ is H.

In some embodiments, one or more L¹ is of the formula

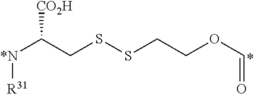

R³¹ is selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};

each R³², R^{32′}, R³³ and R^{33′} are independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl; and

* is a covalent bond. In some embodiments, R³¹ is H.

In some embodiments, one or more L¹ is of the formula

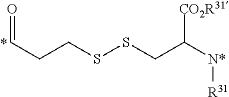

wherein

each R³¹ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};

each R^{31′} is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR^{32a}, —OC(O)R^{32a}, —OC(O)NR^{32a}R^{32a′}, —OS(O)R^{32a}, —OS(O)₂R^{32a}, —SR^{32a}, —S(O)R^{32a}, —S(O)₂R^{32a}, —S(O)NR^{32a}R^{32a′}, —S(O)₂NR^{32a}R^{32a′}—OS(O)NR^{32a}R^{32a′}, —OS(O)₂NR^{32a}R^{32a′}, —NR^{32a}R^{32a′}, —C(O)R^{32a}, —C(O)OR^{32a} or —C(O)NR^{32a}R^{32a′};

each R^{32a}, R^{32a′}, R³², R^{32′}, R³³ and R^{33′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl; and

each * is a covalent bond.

In some embodiments, one or more L¹ is of the formula

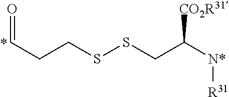

wherein

each R³¹ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};

each R^{31′} is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR^{32a}, —OC(O)R^{32a}, —OC(O)NR^{32a}R^{32a′}, —OS(O)R^{32a}, —OS(O)₂R^{32a}, —SR^{32a}, —S(O)R^{32a}, —S(O)₂R^{32a}, —S(O)NR^{32a}R^{32a′}, —S(O)₂NR^{32a}R^{32a′}—OS(O)NR^{32a}R^{32a′}, —OS(O)₂NR^{32a}R^{32a′}, —NR^{32a}R^{32a′}, —C(O)R^{32a}, —C(O)OR^{32a} or —C(O)NR^{32a}R^{32a′};

each R^{32a}, R^{32a′}, R³², R^{32′}, R³³ and R^{33′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl; and

each * is a covalent bond.

In some embodiments, one or more L¹ is of the formula

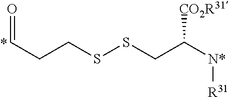

wherein

each R³¹ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};

each R^{31′} is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR^{32a}, —OC(O)R^{32a}, —OC(O)NR^{32a}R^{32a′}, —OS(O)R^{32a}, —OS(O)₂R^{32a}, —SR^{32a}, —S(O)R^{32a}, —S(O)₂R^{32a}, —S(O)NR^{32a}R^{32a′}, —S(O)₂NR^{32a}R^{32a′}—OS(O)NR^{32a}R^{32a′}, —OS(O)₂NR^{32a}R^{32a′}, —NR^{32a}R^{32a′}, —C(O)R^{32a}, —C(O)OR^{32a} or —C(O)NR^{32a}R^{32a′};

each R^{32a}, R^{32a′}, R³², R^{32′}, R³³ and R^{33′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl; and

each * is a covalent bond.

In some embodiments, one or more L¹ is of the formula

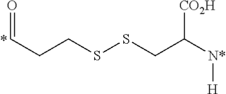

wherein each * is a covalent bond.

In some embodiments, one or more L¹ is of the formula

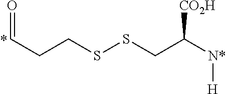

wherein each * is a covalent bond.

In some embodiments, one or more L¹ is of the formula

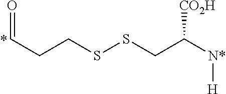

wherein each * is a covalent bond.

In some embodiments, one or more L¹ is of the formula

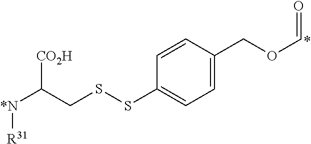

R³¹ is selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};

each R³², R^{32′}, R³³ and R^{33′} are independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl; and

* is a covalent bond. In some embodiments, R³¹ is H.

In some embodiments, one or more L¹ is of the formula

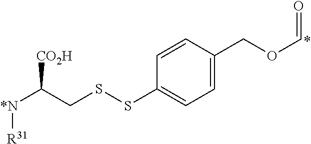

R³¹ is selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};

each R³², R^{32′}, R³³ and R^{33′} are independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl; and

* is a covalent bond. In some embodiments, R³¹ is H.

In some embodiments, one or more L¹ is of the formula

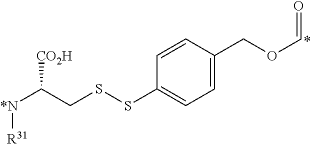

R³¹ is selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};

each R³², R^{32′}, R³³ and R^{33′} are independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl; and

* is a covalent bond. In some embodiments, R³¹ is H.

In some embodiments, one or more L¹ is of the formula

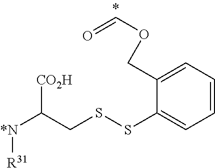

R³¹ is selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};

each R³², R^{32′}, R³³ and R^{33′} are independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl; and

* is a covalent bond. In some embodiments, R³¹ is H.

In some embodiments, one or more L¹ is of the formula

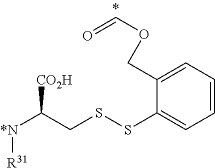

R³¹ is selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};

each R³², R^{32′}, R³³ and R^{33′} are independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl; and

* is a covalent bond. In some embodiments, R³¹ is H.

In some embodiments, one or more L¹ is of the formula

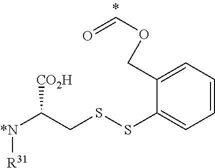

R³¹ is selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)OR³³, —NR³²C(O)NR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};

each R³², R^{32′}, R³³ and R^{33′} are independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl; and

* is a covalent bond. In some embodiments, R³¹ is H.

In some embodiments, one or more L¹ is of the formula

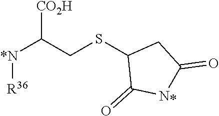

each R³⁶ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁷, —OC(O)R³⁷, —OC(O)NR³⁷R^{37′}, —OS(O)R³⁷, —OS(O)₂R³⁷, —SR³⁷, —S(O)R³⁷, —S(O)₂R³⁷, —S(O)NR³⁷R^{37′}, —S(O)₂NR³⁷R^{37′}, —OS(O)NR³⁷R^{37′}, —OS(O)₂NR³⁷R^{37′}, —NR³⁷R^{37′}, —NR³⁷C(O)R³⁸, —NR³⁷C(O)OR³⁸, —NR³⁷C(O)NR³⁸R^{38′}, —NR³⁷S(O)R³⁸, —NR³⁷S(O)₂R³⁸, —NR³⁷S(O)NR³⁸R^{38′}, —NR³⁷S(O)₂NR³⁸R^{38′}, —C(O)R³⁷, —C(O)OR³⁷ or —C(O)NR³⁷R^{37′};

R³⁷, R^{37′}, R³⁸ and R^{38′ ′} are each independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl; and

* is a covalent bond. In some embodiments, R³⁶ is H.

In some embodiments, one or more L¹ is of the formula

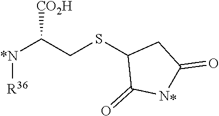

each R³⁶ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁷, —OC(O)R³⁷, —OC(O)NR³⁷R^{37′}, —OS(O)R³⁷, —OS(O)₂R³⁷, —SR³⁷, —S(O)R³⁷, —S(O)₂R³⁷, —S(O)NR³⁷R^{37′}, —S(O)₂NR³⁷R^{37′}, —OS(O)NR³⁷R^{37′}, —OS(O)₂NR³⁷R^{37′}, —NR³⁷R^{37′}, —NR³⁷C(O)R³⁸, —NR³⁷C(O)OR³⁸, —NR³⁷C(O)NR³⁸R^{38′}, —NR³⁷S(O)R³⁸, —NR³⁷S(O)₂R³⁸, —NR³⁷S(O)NR³⁸R^{38′}, —NR³⁷S(O)₂NR³⁸R^{38′}, —C(O)R³⁷, —C(O)OR³⁷ or —C(O)NR³⁷R^{37′};

R³⁷, R^{37′}, R³⁸ and R^{38′} are each independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl; and

* is a covalent bond. In some embodiments, R³⁶ is H.

In some embodiments, one or more L¹ is of the formula

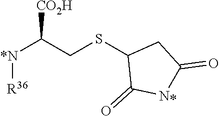

each R³⁶ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁷, —OC(O)R³⁷, —OC(O)NR³⁷R^{37′}, —OS(O)R³⁷, —OS(O)₂R³⁷, —SR³⁷, —S(O)R³⁷, —S(O)₂R³⁷, —S(O)NR³⁷R^{37′}, —S(O)₂NR³⁷R^{37′}, —OS(O)NR³⁷R^{37′}, —OS(O)₂NR³⁷R^{37′}, —NR³⁷R^{37′}, —NR³⁷C(O)R³⁸, —NR³⁷C(O)OR³⁸, —NR³⁷C(O)NR³⁸R^{38′}, —NR³⁷S(O)R³⁸, —NR³⁷S(O)₂R³⁸, —NR³⁷S(O)NR³⁸R^{38′}, —NR³⁷S(O)₂NR³⁸R^{38′}, —C(O)R³⁷, —C(O)OR³⁷ or —C(O)NR³⁷R^{37′};

R³⁷, R^{37′}, R³⁸ and R^{38′} are each independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl; and

* is a covalent bond. In some embodiments, R³⁶ is H.

In some embodiments, one or more L¹ is of the formula

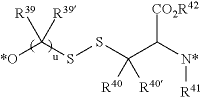

wherein

each R³⁹, R^{39′}, R⁴⁰ and R^{40′} is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁴⁴, —OC(O)R⁴⁴, —OC(O)NR⁴⁴R^{44′}, —OS(O)R⁴⁴, —OS(O)₂R⁴⁴, —SR⁴⁴, —S(O)R⁴⁴, —S(O)₂R⁴⁴, —S(O)NR⁴⁴R^{44′}, —S(O)₂NR⁴⁴R^{44′}, —OS(O)NR⁴⁴R^{44′}, —OS(O)₂NR⁴⁴R^{44′}, —NR⁴⁴R^{44′}, —NR⁴⁴C(O)R⁴⁵, —NR⁴⁴C(O)OR⁴⁵, —NR⁴⁴C(O)NR⁴⁵R^{45′}, —NR⁴⁴S(O)R⁴⁵, —NR⁴⁴S(O)₂R⁴⁵, —NR⁴⁴S(O)NR⁴⁵R^{45′}, —NR⁴⁴S(O)₂NR⁴⁵R^{45′}, —C(O)R⁴⁴, —C(O)OR⁴⁴ or —C(O)NR⁴⁴R^{44′};

each R⁴¹ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁴⁶, —OC(O)R⁴⁶, —OC(O)NR⁴⁶R^{46′}, —OS(O)R⁴⁶, —OS(O)₂R⁴⁶, —SR⁴⁶, —S(O)R⁴⁶, —S(O)₂R⁴⁶, —S(O)NR⁴⁶R^{46′}, —S(O)₂NR⁴⁶R^{46′}, —OS(O)NR⁴⁶R^{46′}, —OS(O)₂NR⁴⁶R^{46′}, —NR⁴⁶R^{46a′}, —NR⁴⁶C(O)R⁴⁷, —NR⁴⁶C(O)OR⁴⁷, —NR⁴⁶C(O)NR⁴⁷R^{47′}, —NR⁴⁶S(O)R⁴⁷, —NR⁴⁶S(O)₂R⁴⁷, —NR⁴⁶S(O)NR⁴⁷R^{47′}, —NR⁴⁶S(O)₂NR⁴⁷R^{47′}, —C(O)R⁴⁶, —C(O)OR⁴⁶ or —C(O)NR⁴⁶R^{46′};

each R⁴² is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR⁴³, —OC(O)R⁴³, —OC(O)NR⁴³R^{43′}, —OS(O)R⁴³, —OS(O)₂R⁴³, —SR⁴³, —S(O)R⁴³, —S(O)₂R⁴³, —S(O)NR⁴³R^{43′}, —S(O)₂NR⁴³R^{43′}, —OS(O)NR⁴³R^{43′}, —OS(O)₂NR⁴³R^{43′}, —NR⁴³R^{43′}, —C(O)R⁴³, —C(O)OR⁴³ or —C(O)NR⁴³R^{43′};

each R⁴³, R^{43′}, R⁴⁴, R^{44′}, R⁴⁵, R^{45′}, R⁴⁶, R^{46′}, R⁴⁷ and R^{47′} is independently selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl;

u is 1, 2, 3 or 4; and

each * is a covalent bond. In some embodiments, u is 2. In some embodiments, u is 3. In some embodiments, R³⁹ and R^{39′} are H. In some embodiments, two R³⁹ and R^{39′} attached to the same carbon atom are —CH₃. In some embodiments, R⁴⁰ and R^{40′} are H. In some embodiments, R⁴⁰ and R^{40′} are —CH₃. In some embodiments, R⁴¹ is H. In some embodiments, R⁴² is H. In some embodiments each R³⁹ and R^{39′} is H, R⁴⁰ and R^{40′} are —CH₃, R⁴¹ is H, and R⁴² is H.

In some embodiments one or more L¹ is of the formula

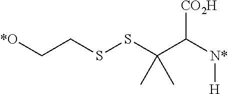

wherein each * is a covalent bond.

In some embodiments one or more L¹ is of the formula

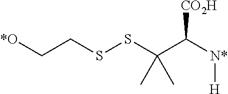

wherein each * is a covalent bond.

In some embodiments one or more L¹ is of the formula

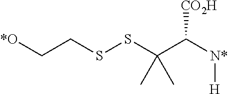

wherein each * is a covalent bond.

In some embodiments one or more L¹ is of the formula

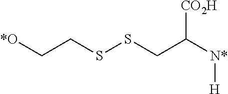

wherein each * is a covalent bond.

In some embodiments one or more L¹ is of the formula

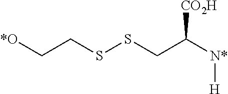

wherein each * is a covalent bond.

In some embodiments one or more L¹ is of the formula

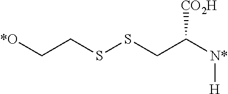

wherein each * is a covalent bond.

In some embodiments, one or more L¹ is of the formula

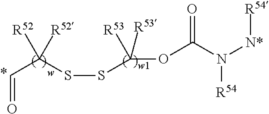

each R⁵², R^{52′}, R⁵³ and R^{53′} is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁵⁵, —OC(O)R⁵⁵, —OC(O)NR⁵⁵R^{55′}, —OS(O)R⁵⁵, —OS(O)₂R⁵⁵, —SR⁵⁵, —S(O)R⁵⁵, —S(O)₂R⁵⁵, —S(O)NR⁵⁵R^{55′}, —S(O)₂NR⁵⁵R^{55′}, —OS(O)NR⁵⁵R^{55′}, —OS(O)₂NR⁵⁵R^{55′}, —NR⁵⁵R^{55′}, —NR⁵⁵C(O)R⁵⁶, —NR⁵⁵C(O)OR⁵⁶, —NR⁵⁵C(O)NR⁵⁶R^{56′}, —NR⁵⁵S(O)R⁵⁶, —NR⁵⁵S(O)₂R⁵⁶, —NR⁵⁵S(O)NR⁵⁶R^{56′}, —NR⁵⁵S(O)₂NR⁵⁶R^{56′}, —C(O)R⁵⁵, —C(O)OR⁵⁵ or —C(O)NR⁵⁵R^{55′};

each R⁵⁴ and R^{54′} is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁵⁷, —OC(O)R⁵⁷, —OC(O)NR⁵⁷R^{57′}, —OS(O)R⁵⁷, —OS(O)₂R⁵⁷, —SR⁵⁷, —S(O)R⁵⁷, —S(O)₂R⁵⁷, —S(O)NR⁵⁷R^{57′}, —S(O)₂NR⁵⁷R^{57′}, —OS(O)NR⁵⁷R^{57′}, —OS(O)₂NR⁵⁷R^{57′}, —NR⁵⁷R^{57′}, —NR⁵⁷C(O)R⁵⁸, —NR⁵⁷C(O)OR⁵⁸, —NR⁵⁷C(O)NR⁵⁸R^{58′}, —NR⁵⁷S(O)R⁵⁸, —NR⁵⁷S(O)₂R⁵⁸, —NR⁵⁷S(O)NR⁵⁸R^{58′}, —NR⁵⁷S(O)₂NR⁵⁸R^{58′}, —C(O)R⁵⁷, —C(O)OR⁵⁷ or —C(O)NR⁵⁷R^{57′};

R⁵⁵, R^{55′}, R⁵⁶, R^{56′} R⁵⁷, R^{57′}, R⁵⁸ and R^{58′} are each independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl;

w is 1, 2, 3 or 4;

w1 is 1, 2, 3 or 4; and

* is a covalent bond.

In some embodiments, w is 2. In some embodiments, w1 is 2. In some embodiments, w is 2 and w1 is 2. In some embodiments, each of R⁵², R^{52′}, R⁵³ and R^{53′} is H. In some embodiments, two of R⁵² and R^{52′} attached to the same carbon atom are —CH₃. In some embodiments, two of R⁵³ and R^{53′} attached to the same carbon atom are —CH₃. In some embodiments, two of R⁵² and R^{52′} attached to the same carbon atom are —CH₃, and two of R⁵³ and R^{53′} attached to the same carbon atom are —CH₃.

In some embodiments, one or more L¹ is of the formula

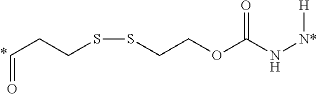

wherein each * is a covalent bond.
In some embodiments, one or more L¹ is of the formula

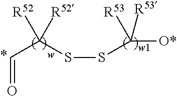

each R⁵², R^{52′}, R⁵³ and R^{53′} is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁵⁵, —OC(O)R⁵⁵, —OC(O)NR⁵⁵R^{55′}, —OS(O)R⁵⁵, —OS(O)₂R⁵⁵, —SR⁵⁵, —S(O)R⁵⁵, —S(O)₂R⁵⁵, —S(O)NR⁵⁵R^{55′}, —S(O)₂NR⁵⁵R^{55′}, —OS(O)NR⁵⁵R^{55′}, —OS(O)₂NR⁵⁵R^{55′}, —NR⁵⁵R^{55′}, —NR⁵⁵C(O)R⁵⁶, —NR⁵⁵C(O)OR⁵⁶, —NR⁵⁵C(O)NR⁵⁶R^{56′}, —NR⁵⁵S(O)R⁵⁶, —NR⁵⁵S(O)₂R⁵⁶, —NR⁵⁵S(O)NR⁵⁶R^{56′}, —NR⁵⁵S(O)₂NR⁵⁶R^{56′}, —C(O)R⁵⁵, —C(O)OR⁵⁵ or —C(O)NR⁵⁵R^{55′};

R⁵⁵, R^{55′}, R⁵⁶ and R^{56′} are each independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl;

w is 1, 2, 3 or 4;

w1 is 1, 2, 3 or 4; and

* is a covalent bond.

In some embodiments, w is 2. In some embodiments, w1 is 2. In some embodiments, w is 2 and w1 is 2. In some embodiments, each of R⁵², R^{52′}, R⁵³ and R^{53′} is H. In some embodiments, two of R⁵² and R^{52′} attached to the same carbon atom are —CH₃. In some embodiments, two of R⁵³ and R^{53′} attached to the same carbon atom are —CH₃. In some embodiments, two of R⁵² and R^{52′} attached to the same carbon atom are —CH₃, and two of R⁵³ and R^{53′} attached to the same carbon atom are —CH₃.

In some embodiments, one or more L¹ is of the formula

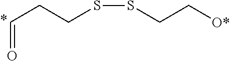

wherein each * is a covalent bond.

As used herein, L² can be any group covalently attaching portions of the linker to the binding ligand, portions of the linker to other portions of the linker, or portions of the linker to D¹. It will be understood that the structure of L² is not particularly limited in any way. It will be further understood that L² can comprise numerous functionalities well known in the art to covalently attach portions of the linker to the binding ligand, portions of the linker to other portions of the linker, or portions of the linker to D¹, including but not limited to, alkyl groups, ether groups, amide groups, carboxy groups, sulfonate groups, alkenyl groups, alkynyl groups, cycloalkyl groups, aryl groups, heterocycloalkyl, heteroaryl groups, and the like. In some embodiments, L² is a linker of the formula II

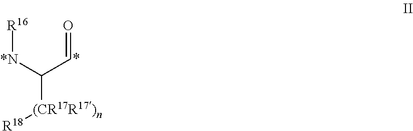

wherein

R¹⁶ is selected from the group consisting of H, D, C₁-C₆ alkyl, C₂-C₆ alkenyl, C₂-C₆ alkynyl, —C(O)R¹⁹, —C(O)OR¹⁹ and —C(O)NR¹⁹R^{19′}, wherein each hydrogen atom in C₁-C₆ alkyl, C₂-C₆ alkenyl and C₂-C₆ alkynyl is independently optionally substituted by halogen, C₁-C₆ alkyl, C₂-C₆ alkenyl, and C₂-C₆ alkynyl, —OR²⁰, —OC(O)R²⁰, —OC(O)NR²⁰R^{20′}, —OS(O)R²⁰, —OS(O)₂R²⁰, —SR²⁰, —S(O)R²⁰, —S(O)₂R²⁰, —S(O)NR²⁰R^{20′}, —S(O)₂NR²⁰R^{20′}, —OS(O)NR²⁰R^{20′}, —OS(O)₂NR²⁰R^{20′}, —NR²⁰R^{20′}, —NR²⁰C(O)R²¹, —NR²⁰C(O)OR²¹, —NR²⁰C(O)NR²¹R^{21′}, —NR²⁰S(O)R²¹, —NR²⁰S(O)₂R²¹, —NR²⁰S(O)NR²¹R^{2r}, —NR²⁰S(O)₂NR²¹R^{21′}, —C(O)R²⁰, —C(O)OR²⁰ or —C(O)NR²⁰R^{20′};

each R¹⁷ and R^{17′} is independently selected from the group consisting of H, D, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²², —OC(O)R²², —OC(O)NR²²R^{22′}, —OS(O)R²², —OS(O)₂R²², —SR²², —S(O)R²², —S(O)₂R²², —S(O)NR²²R^{22′}, —S(O)₂NR²²R^{22′}, —OS(O)NR²²R^{22′}, —OS(O)₂NR²²R^{22′}, —NR²²R^{22′}, —NR²²C(O)R²³, —NR²²C(O)OR²³, —NR²²C(O)NR²³R^{23′}, —NR²²S(O)R²³, —NR²²S(O)₂R²³, —NR²²S(O)NR²³R^{23′}, —NR²²S(O)₂NR²³R^{23′}, —C(O)R²², —C(O)OR²², and —C(O)NR²²R^{22′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, C₂-C₆ alkynyl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′}; or R¹⁷ and R^{17′} may combine to form a C₄-C₆ cycloalkyl or a 4- to 6-membered heterocycle, wherein each hydrogen atom in C₄-C₆ cycloalkyl or 4- to 6-membered heterocycle is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′};

R¹⁸ is selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26″})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};

each R¹⁹, R^{19′}, R²⁰, R^{20′}, R²¹, R^{21′}, R²², R^{22′}, R²³, R^{23′}, R²⁴, R^{24′}, R²⁵, R^{25′}, R²⁶, R^{26′}, R^{26″}, R²⁹, R^{29′}, R³⁰ and R^{30′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;

R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}— (sugar) and —(CH₂)ₚ(OCH₂CH₂CH₂)_{q}(sugar);

R²⁸ is H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;

n is 1, 2, 3, 4 or 5;

p is 1, 2, 3, 4 or 5;

q is 1, 2, 3, 4 or 5; and

* is a covalent bond.

It will be appreciate that when L² is described according to the formula III, that both the R- and S-configurations are contemplated. In some embodiments, L² is of the formula IIa or IIb

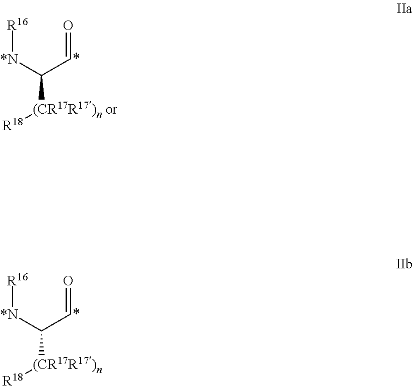

where each of R¹⁶, R¹⁷, R^{17′}, R¹⁸, n and * are as defined for the formula II.

In some embodiments, each L² is selected from the group consisting of

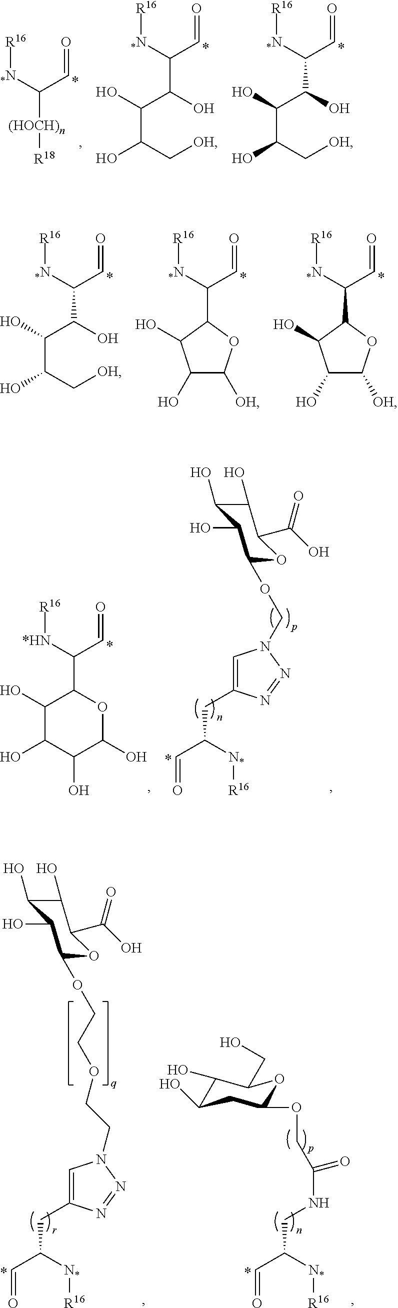

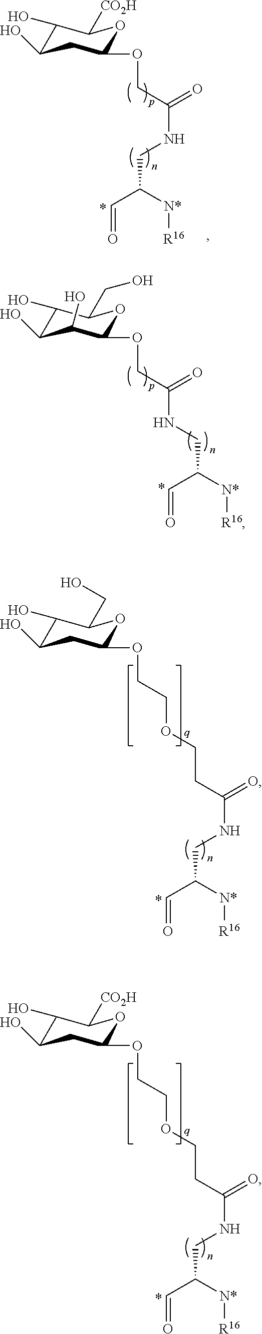

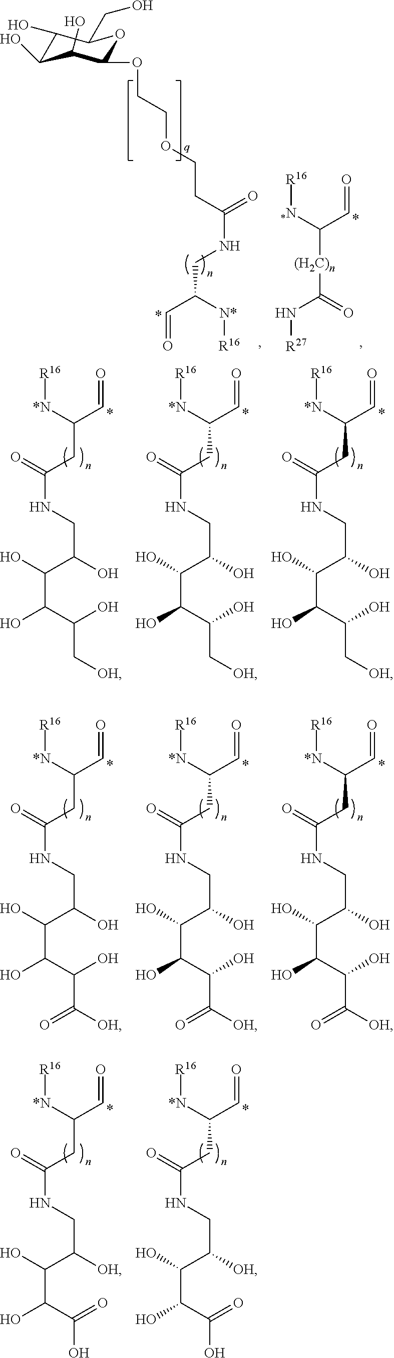

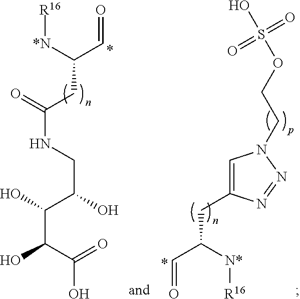

and combinations thereof,
wherein

R¹⁶ is selected from the group consisting of H, D, C₁-C₆ alkyl, C₂-C₆ alkenyl, C₂-C₆ alkynyl, —C(O)R¹⁹, —C(O)OR¹⁹ and —C(O)NR¹⁹R^{19′}, wherein each hydrogen atom in C₁-C₆ alkyl, C₂-C₆ alkenyl and C₂-C₆ alkynyl is independently optionally substituted by halogen, C₁-C₆ alkyl, C₂-C₆ alkenyl, and C₂-C₆ alkynyl, —OR²⁰, —OC(O)R²⁰, —OC(O)NR²⁰R^{20′}, —OS(O)R²⁰, —OS(O)₂R²⁰, —SR²⁰, —S(O)R²⁰, —S(O)₂R²⁰, —S(O)NR²⁰R^{20′}, —S(O)₂NR²⁰R^{20′}, —OS(O)NR²⁰R^{20′}, —OS(O)₂NR²⁰R^{20′}, —NR²⁰R^{20′}, —NR²⁰C(O)R²¹, —NR²⁰C(O)OR²¹, —NR²⁰C(O)NR²¹R^{21′}, —NR²⁰S(O)R²¹, —NR²⁰S(O)₂R²¹, —NR²⁰S(O)NR²¹R^{21′}, —NR²⁰S(O)₂NR²¹R^{21′}, —C(O)R²⁰, —C(O)OR²⁰ or —C(O)NR²⁰R^{20′};

R¹⁸ is selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26″})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};

each R¹⁹, R^{19′}, R²⁰, R^{20′}, R²¹, R^{21′}, R²⁶, R^{26′}, R^{26″}, R²⁹, R^{29′}, R³⁰ and R^{30′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;

R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}— (sugar) and —(CH₂)ₚ(OCH₂CH₂CH₂)_{q}(sugar);

R²⁸ is H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;

n is 1, 2, 3, 4 or 5;

p is 1, 2, 3, 4 or 5;

q is 1, 2, 3, 4 or 5; and

* is a covalent bond.

In some embodiments, each L² is selected from the group consisting of

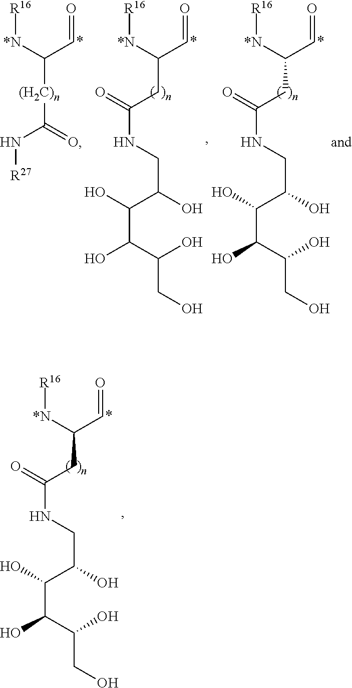

wherein R¹⁶ is defined as described herein, and * is a covalent bond.

In some embodiments, R¹⁶ is H. In some embodiments, R¹⁸ is selected from the group consisting of H, 5- to 7-membered heteroaryl, —OR²⁶, —NR²⁶C(O)R²⁷, —NR²⁶C(O)NR²⁷R^{27′}, —NR²⁶C(═NR^{26′}′)NR²⁷R^{27′}, and —C(O)NR²⁶R^{26′}, wherein each hydrogen atom 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};

each R²⁶, R^{26′}, R^{26″}, R²⁹, R^{29′}, R³⁰ and R^{30′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;

R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}— (sugar) and —(CH₂)ₚ(OCH₂CH₂CH₂)_{q}(sugar);

R²⁸ is a H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;

n is 1, 2, 3, 4 or 5;

p is 1, 2, 3, 4 or 5;

q is 1, 2, 3, 4 or 5; and

* is a covalent bond.

In some embodiments, R¹⁸ is selected from the group consisting of H, 5- to 7-membered heteroaryl, —OR²⁶, —NR²⁶C(O)R²⁷, —NR²⁶C(O)NR²⁷R^{27′}, —NR²⁶C(═NR²⁶)NR²⁷R^{27′}, and —C(O)NR²⁶R^{26′,} wherein each hydrogen atom 5- to 7-membered heteroaryl is independently optionally substituted by —(CH₂)ₚOR²⁸, —OR²⁹, —(CH₂)ₚOS(O)₂OR²⁹ and —OS(O)₂OR²⁹;

each R²⁶, R^{26′}, R^{26″} and R²⁹ is independently H or C₁-C₇ alkyl, wherein each hydrogen atom in C₁-C₇ alkyl is independently optionally substituted by halogen, —OH, —SH, —NH₂ or —CO₂H;

R²⁷ and R^{27′} are each independently selected from the group consisting of H, —(CH₂)ₚ(sugar), —(CH₂)ₚ(OCH₂CH₂)_{q}(sugar) and —(CH₂)ₚ(OCH₂CH₂CH₂)_{q}(sugar);

R²⁸ is H or sugar;

n is 1, 2, 3, 4 or 5;

p is 1, 2, 3, 4 or 5;

q is 1, 2, 3, 4 or 5; and

* is a covalent bond.

In some embodiments, each L² is selected from the group consisting of

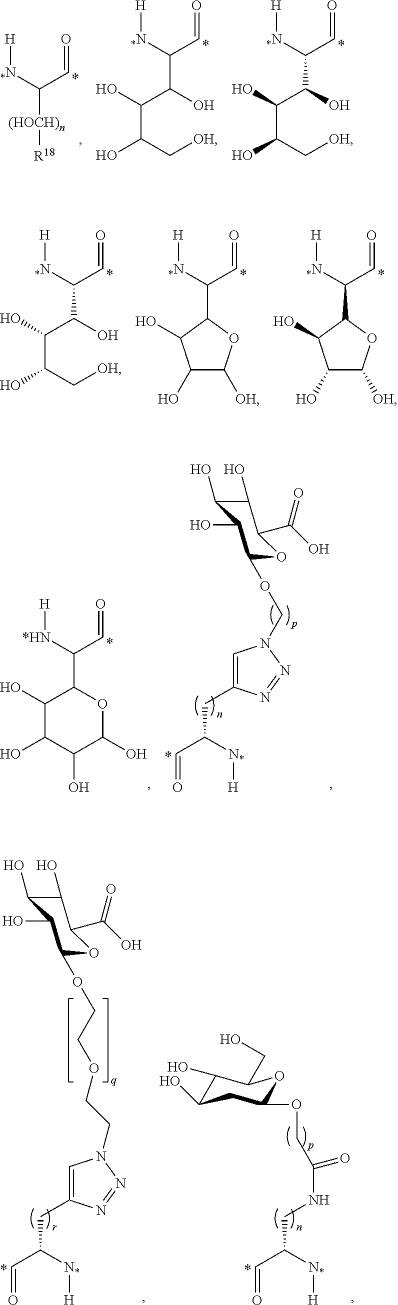

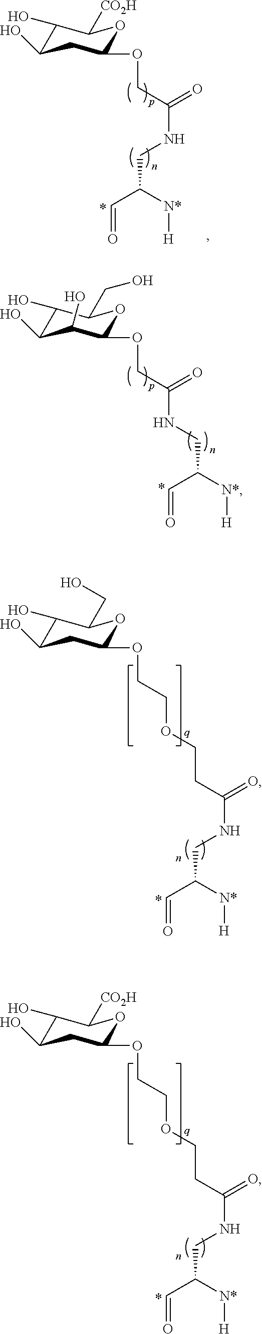

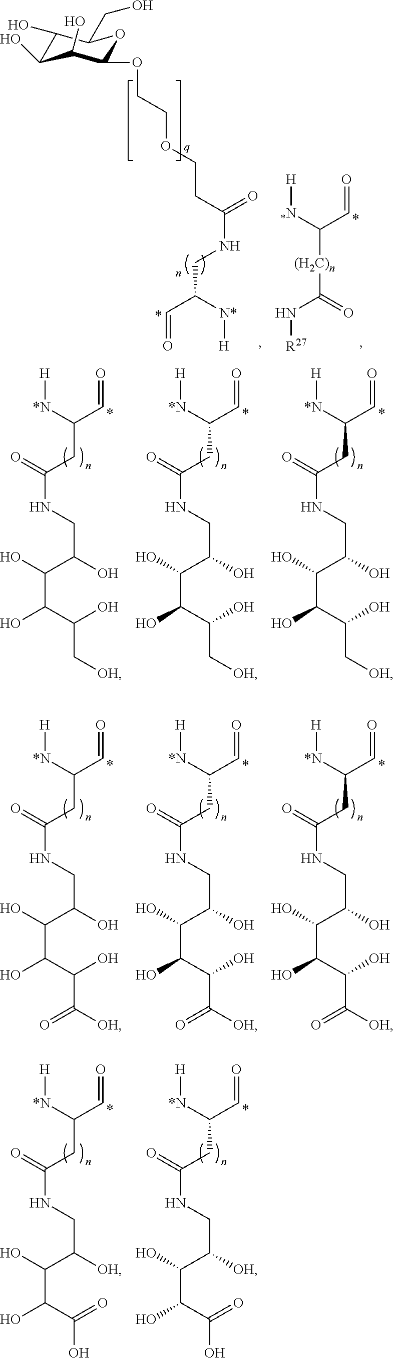

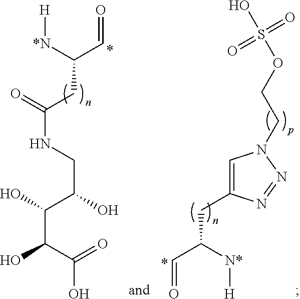

and combinations thereof,

wherein

R¹⁸ is selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR^{26″})NR²⁷R^{27′}, —NR²⁶S(O)R²⁷, —NR²⁶S(O)₂R²⁷, —NR²⁶S(O)NR²⁷R^{27′}, —NR²⁶S(O)₂NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};

each R²⁶, R^{26′}, R^{26″}, R²⁹, R^{29′}, R³⁰ and R^{30′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;

R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}— (sugar) and —(CH₂)ₚ(OCH₂CH₂CH₂)_{q}(sugar);

R²⁸ is a H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;

n is 1, 2, 3, 4 or 5;

p is 1, 2, 3, 4 or 5;

q is 1, 2, 3, 4 or 5; and

* is a covalent bond.

In some embodiments, R¹⁸ is selected from the group consisting of H, 5- to 7-membered heteroaryl, —OR²⁶, —NR²⁶C(O)R²⁷, —NR²⁶C(O)NR²⁷R^{27′}, —NR²⁶C(═NR^{26″})NR²⁷R^{27′}, and —C(O)NR²⁶R^{26′}, wherein each hydrogen atom 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};

each R²⁶, R^{26′}, R^{26″}, R²⁹, R^{29′}, R³⁰ and R^{30′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;

R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}— (sugar) and —(CH₂)ₚ(OCH₂CH₂CH₂)_{q}(sugar);

R²⁸ is a H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;

n is 1, 2, 3, 4 or 5;

p is 1, 2, 3, 4 or 5;

q is 1, 2, 3, 4 or 5; and

* is a covalent bond.

In some embodiments, R¹⁸ is selected from the group consisting of H, 5- to 7-membered heteroaryl, —OR²⁶, —NR²⁶C(O)R²⁷, —NR²⁶C(O)NR²⁷R^{27′}, —NR²⁶C(═NR²⁶)NR²⁷R^{27′}, and —C(O)NR²⁶R^{26′}, wherein each hydrogen atom 5- to 7-membered heteroaryl is independently optionally substituted by —(CH₂)ₚOR²⁸, —OR²⁹, —(CH₂)ₚOS(O)₂OR²⁹ and —OS(O)₂OR²⁹,

each R²⁶, R^{26′}, R^{26″} and R²⁹ is independently H or C₁-C₇ alkyl, wherein each hydrogen atom in C₁-C₇ alkyl is independently optionally substituted by halogen, —OH, —SH, —NH₂ or —CO₂H;

R²⁷ and R^{27′} are each independently selected from the group consisting of H, —(CH₂)ₚ(sugar), —(CH₂)ₚ(OCH₂CH₂)_{q}(sugar) and —(CH₂)ₚ(OCH₂CH₂CH₂)_{q}(sugar);

R²⁸ is H or sugar;

n is 1, 2, 3, 4 or 5;

p is 1, 2, 3, 4 or 5;

q is 1, 2, 3, 4 or 5; and

* is a covalent bond.

AA is an amino acid as defined herein. In certain embodiments, AA is a naturally occurring amino acid. In certain embodiments, AA is in the L-form. In certain embodiments, AA is in the D-form. It will be appreciated that in certain embodiments, the conjugates described herein will comprise more than one amino acid as portions of the linker, and the amino acids can be the same or different, and can be selected from a group of amino acids. It will be appreciated that in certain embodiments, the conjugates described herein will comprise more than one amino acid as portions of the linker, and the amino acids can be the same or different, and can be selected from a group of amino acids in D- or L-form. In some embodiments, each AA is independently selected from the group consisting of L-lysine, L-asparagine, L-threonine, L-serine, L-isoleucine, L-methionine, L-proline, L-histidine, L-glutamine, L-arginine, L-glycine, L-aspartic acid, L-glutamic acid, L-alanine, L-valine, L-phenylalanine, L-leucine, L-tyrosine, L-cysteine, L-tryptophan, L-phosphoserine, L-sulfo-cysteine, L-arginosuccinic acid, L-hydroxyproline, L-phosphoethanolamine, L-sarcosine, L-taurine, L-carnosine, L-citrulline, L-anserine, L-1,3-methyl-histidine, L-alpha-amino-adipic acid, D-lysine, D-asparagine, D-threonine, D-serine, D-isoleucine, D-methionine, D-proline, D-histidine, D-glutamine, D-arginine, D-glycine, D-aspartic acid, D-glutamic acid, D-alanine, D-valine, D-phenylalanine, D-leucine, D-tyrosine, D-cysteine, D-tryptophan, D-citrulline and D-carnosine.

In some embodiments, each AA is independently selected from the group consisting of L-asparagine, L-arginine, L-glycine, L-aspartic acid, L-glutamic acid, L-glutamine, L-cysteine, L-alanine, L-valine, L-leucine, L-isoleucine, L-citrulline, D-asparagine, D-arginine, D-glycine, D-aspartic acid, D-glutamic acid, D-glutamine, D-cysteine, D-alanine, D-valine, D-leucine, D-isoleucine and D-citrulline. In some embodiments, each AA is independently selected from the group consisting of Asp, Arg, Val, Ala, Cys and Glu.

In certain embodiments, L can be of the formula selected from the group consisting of

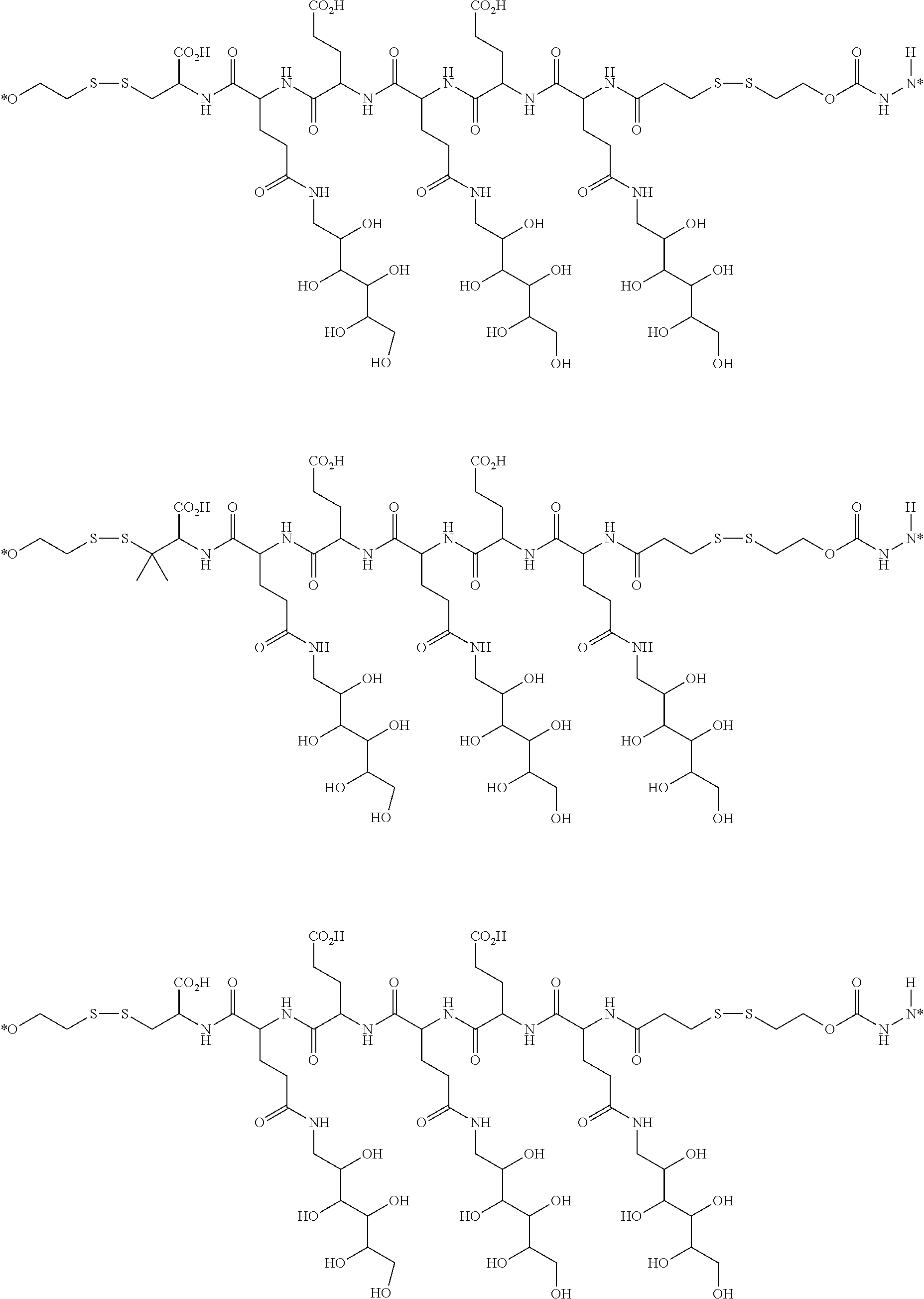

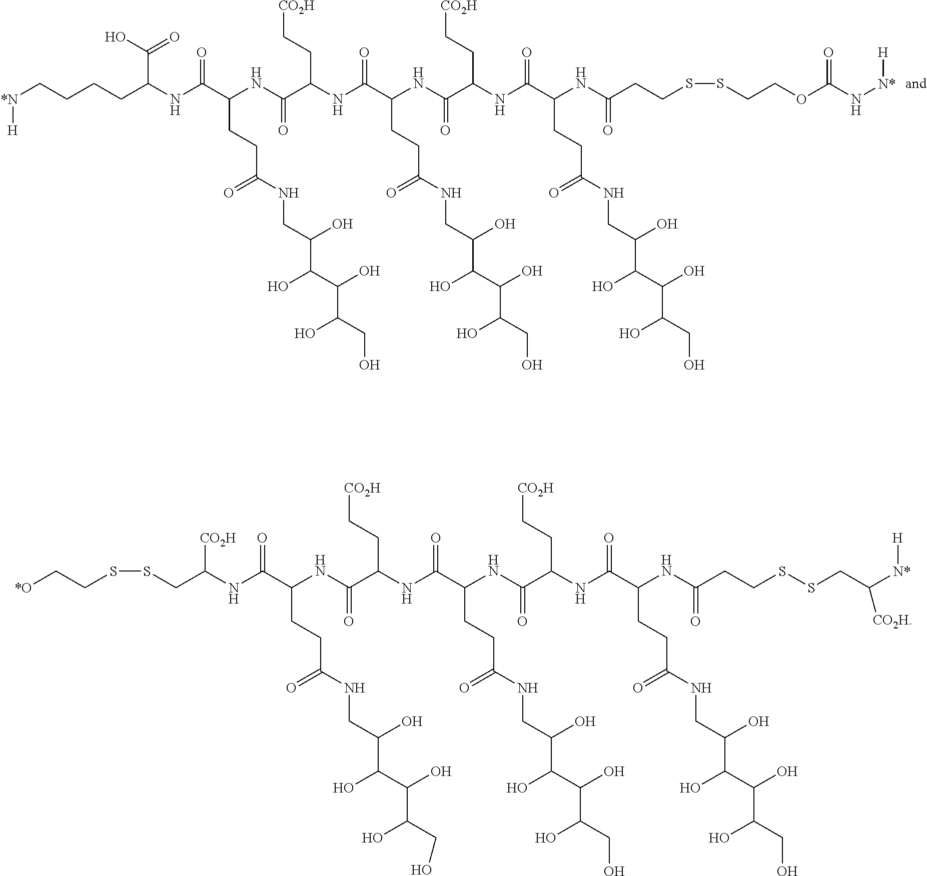

The drug (also known herein as D¹) used in connection with any of the conjugates described herein can be any molecule capable of modulating or otherwise modifying cell function, including pharmaceutically active compounds. Suitable molecules can include, but are not limited to peptides, oligopeptides, retro-inverso oligopeptides, proteins, protein analogs in which at least one non-peptide linkage replaces a peptide linkage, apoproteins, glycoproteins, enzymes, coenzymes, enzyme inhibitors, amino acids and their derivatives, receptors and other membrane proteins; antigens and antibodies thereto; haptens and antibodies thereto; hormones, lipids, phospholipids, liposomes; toxins; antibiotics; analgesics; bronchodilators; beta-blockers; antimicrobial agents; antihypertensive agents; cardiovascular agents including antiarrhythmics, cardiac glycosides, antianginals and vasodilators; central nervous system agents including stimulants, psychotropics, antimanics, and depressants; antiviral agents; antihistamines; cancer drugs including chemotherapeutic agents; tranquilizers; anti-depressants; H-2 antagonists; anticonvulsants; antinauseants; prostaglandins and prostaglandin analogs; muscle relaxants; anti-inflammatory substances; stimulants; decongestants; antiemetics; diuretics; antispasmodics; antiasthmatics; anti-Parkinson agents; expectorants; cough suppressants; mucolytics; and mineral and nutritional additives.

Further, the D¹ can be any drug known in the art which is cytotoxic, enhances tumor permeability, inhibits tumor cell proliferation, promotes apoptosis, decreases anti-apoptotic activity in target cells, is used to treat diseases caused by infectious agents, enhances an endogenous immune response directed to the pathogenic cells, or is useful for treating a disease state caused by any type of pathogenic cell. Drugs suitable for use in accordance with the conjugates described herein include adrenocorticoids and corticosteroids, alkylating agents, antiandrogens, antiestrogens, androgens, aclamycin and aclamycin derivatives, estrogens, antimetabolites such as cytosine arabinoside, purine analogs, pyrimidine analogs, and methotrexate, busulfan, carboplatin, chlorambucil, cisplatin and other platinum compounds, tamoxiphen, taxol, paclitaxel, paclitaxel derivatives, Taxotere®, cyclophosphamide, daunomycin, daunorubicin, doxorubicin, rhizoxin, T2 toxin, plant alkaloids, prednisone, hydroxyurea, teniposide, mitomycins, discodermolides, microtubule inhibitors, epothilones, tubulysin, cyclopropyl benz[e]indolone, seco-cyclopropyl benz[e]indolone, 0-Ac-seco-cyclopropyl benz[e]indolone, bleomycin and any other antibiotic, nitrogen mustards, nitrosureas, vincristine, vinblastine, analogs and derivative thereof such as deacetylvinblastine monohydrazide, and other vinca alkaloids, including those described in PCT international publication No. WO 2007/022493, the disclosure of which is incorporated herein by reference, colchicine, colchicine derivatives, allocolchicine, thiocolchicine, trityl cysteine, Halicondrin B, dolastatins such as dolastatin 10, amanitins such as α-amanitin, camptothecin, irinotecan, and other camptothecin derivatives thereof, maytansines, geldanamycin and geldanamycin derivatives, estramustine, nocodazole, MAP4, colcemid, inflammatory and proinflammatory agents, peptide and peptidomimetic signal transduction inhibitors, and any other art-recognized drug or toxin. Other drugs that can be used as D¹ in conjugates described herein include penicillins, cephalosporins, vancomycin, erythromycin, clindamycin, rifampin, chloramphenicol, aminoglycoside antibiotics, gentamicin, amphotericin B, acyclovir, trifluridine, ganciclovir, zidovudine, amantadine, ribavirin, and any other art-recognized antimicrobial compound.

In other embodiments, the D¹ is a drug selected from the group consisting of a vinca alkaloid, such as DAVLBH, a cryptophycin, bortezomib, thiobortezomib, a tubulysin, aminopterin, rapamycin, paclitaxel, docetaxel, doxorubicin, daunorubicin, everolimus, α-amanatin, verucarin, didemnin B, geldanomycin, purvalanol A, ispinesib, budesonide, dasatinib, an epothilone, a maytansine, and a tyrosine kinase inhibitor, including analogs and derivatives of the foregoing.

In some embodiments, D¹ can be a tubulysin. Natural tubulysins are generally linear tetrapeptides consisting of N-methyl pipecolic acid (Mep), isoleucine (Ile), an unnatural aminoacid called tubuvaline (Tuv), and either an unnatural aminoacid called tubutyrosine (Tut, an analog of tyrosine) or an unnatural aminoacid called tubuphenylalanine (Tup, an analog of phenylalanine).

In some embodiments, D¹ is a tetrapeptide of the formula III

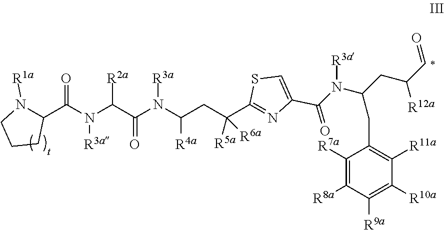

wherein

R^{1a}, R^{3a}, R^{3a′} and R^{3a″} are each independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR^{13a}, —OC(O)R^{13a}, —OC(O)NR^{13a}R^{13a′}, OS(O)R^{13a}, OS(O)₂R^{13a}, SR^{13a}, —SC(O)R^{13a}, —S(O)R^{13a}, —S(O)₂R^{13a}, —S(O)₂OR^{13a}, —S(O)NR^{13a}R^{13a′}, —S(O)₂NR^{13a}R^{13a′}, —OS(O)NR^{13a}R^{13a′}, —OS(O)₂NR^{13a}R^{13a′}, —NR^{13a}R^{13a′}, —NR^{13a}C(O)R^{14a}, —NR^{13a}C(O)OR^{14a}, —NR^{13a}C(O)NR^{14a}R^{14a′}, —NR^{13a}S(O)R^{14a}, —NR^{13a}S(O)₂R^{14a}, —NR^{13a}S(O)NR^{13a}R¹⁴a′, —NR^{13a}S(O)₂NR^{14a}R^{14a′}, —P(O)(OR^{13a})₂, —C(O)R^{13a}, —C(O)OR^{13a} or —C(O)NR^{13a}R^{13a′};

R^{2a}, R^{4a} and R^{12a} are each independently selected from the group consisting of H, D, C₁-C₆ alkyl, C₂-C₆ alkenyl, C₂-C₆ alkynyl;

R^{5a} and R^{6a} are each independently selected from the group consisting of H, D, halogen, C₁-C₆ alkyl, C₂-C₆ alkenyl, C₂-C₆ alkynyl, —OR^{15a}, —SR^{15a}, —OC(O)R^{15a}, —OC(O)NR^{15a}R¹⁵a′, and —NR^{15 a}R^{15a′}, wherein each hydrogen atom in C₁-C₆ alkyl, C₂-C₆ alkenyl and C₂-C₆ alkynyl is independently optionally substituted by halogen, —OR¹⁶a, —SR^{16a}, —NR^{16a}R^{16a″}, —C(O)R^{16a}, —C(O)OR^{16a} or —C(O)NR^{16a}R^{16a′}; or R^{5a} and R^{6a} taken together with the carbon atom to which they are attached form a —C(O)—;

R^{7a}, R^{8a}, R^{9a}, R^{10a} and R^{11a} is independently selected from the group consisting of H, D, halogen, C₁-C₆ alkyl, C₂-C₆ alkenyl, C₂-C₆ alkynyl, —CN, —NO₂, —NCO, —OR^{17a}, —SR^{17a}, —S(O)₂OR¹⁷a, —NR^{17a}R^{17a′}, —P(O)(OR^{17a})₂, —C(O)R^{17a}, —C(O)OR^{17a} and —C(O)NR^{17a}R^{17a′}, wherein each hydrogen atom in C₁-C₆ alkyl, C₂-C₆ alkenyl and C₂-C₆ alkynyl is independently optionally substituted by halogen, —OR^{18a}, —SR¹⁸a, —NR^{18a}R^{18a′}, —C(O)R^{18a}, —C(O)OR^{18a}, or —C(O)NR^{18a}R^{18a′};

each R^{13a}, R^{13a′}, R^{14a}, R^{14a′}, R^{15a}, R^{15a′}, R^{16a}, R^{16a′}, R^{17a} and R^{17a′} is independently selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;

each R^{18a} and R^{18a′} is independently selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl —C(O)R^{19a}, —P(O)(OR^{19a})₂, and —S(O)₂OR^{19a},

each R¹⁹ is independently selected from H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl; and

t is 1, 2 or 3; and

* is a covalent bond.

In some embodiments, D¹ is a of the formula IIIa

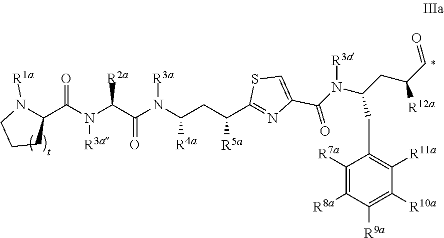

wherein R^{1a}, R^{2a}, R^{3a}, R^{3a′}, R^{3a″}, R^{4a}, R^{5a}, R^{7a}, R^{8a}, R^{9a}, R^{10a}, R^{11a}, and R^{12a} are as described in formula III, and * is a covalent bond.

In another embodiment, naturally occurring tubulysins, and analogs and derivatives thereof, of the following general formula IIIb

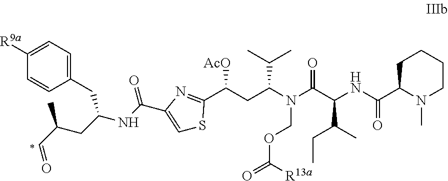

wherein R^{9a} and R^{13a} are as described in formula III, and * is a covalent bond. Conjugates of each of the preceding tubulysins are described herein.

In another embodiment, conjugates of naturally occurring tubulysins of the following general formula are described by the formula IIIc

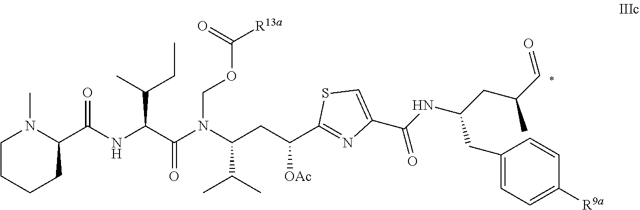

| Factor | R^{13a} | R^{9a} |
|---|---|---|
| A | (CH₃)₂CHCH₂ | OH |
| B | CH₃(CH₂)₂ | OH |
| C | CH₃CH₂ | OH |
| D | (CH₃)₂CHCH₂ | H |
| E | CH₃(CH₂)₂ | H |
| F | CH₂CH₃ | H |
| G | (CH₃)₂C═CH | OH |
| H | CH₃ | H |
| I | CH₃ | OH |

and* is a covalent bond.

In certain embodiments, the disclosure provides a conjugate of the formula selected from the group consisting of

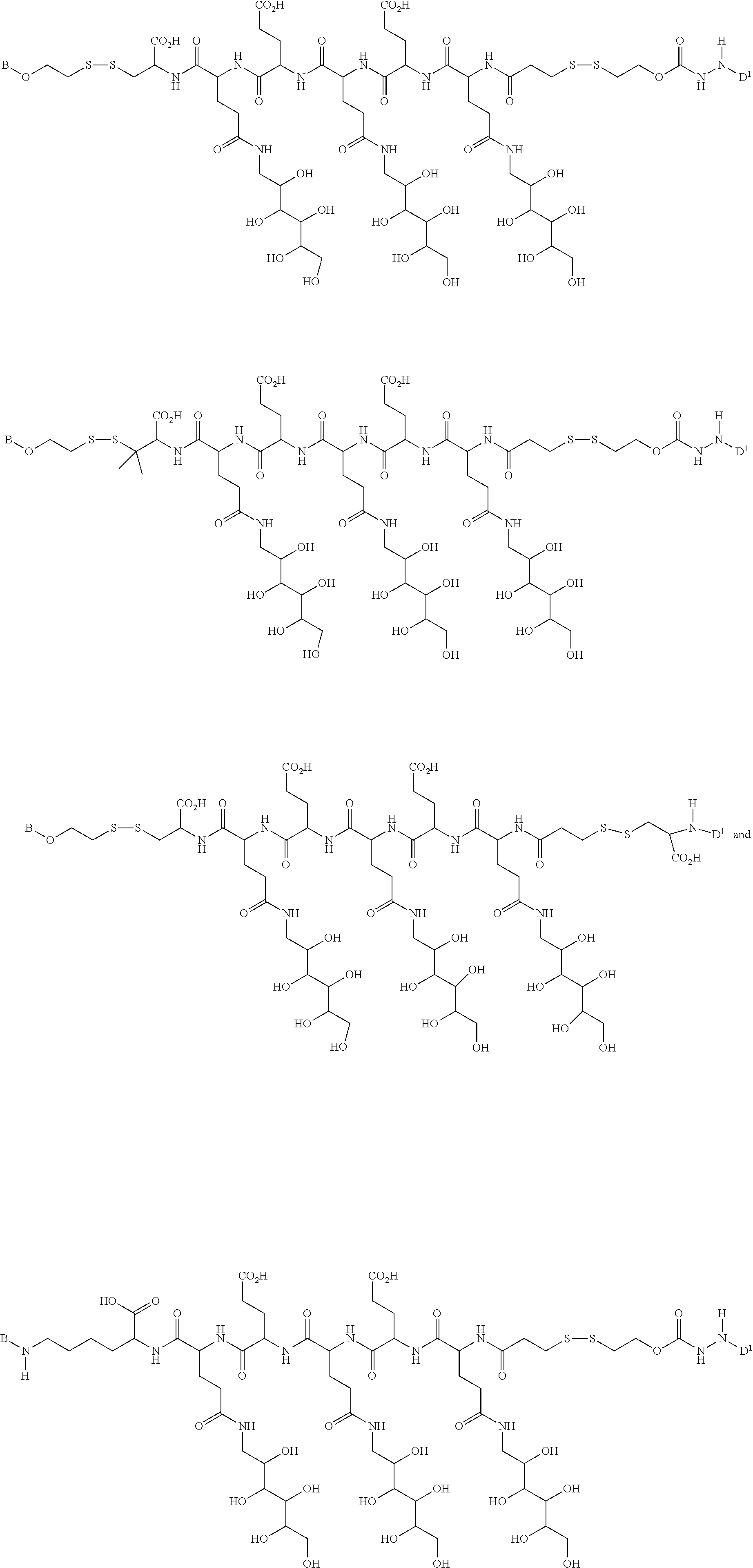

wherein B and D¹ are as described herein, or a pharmaceutically acceptable salt thereof.

The conjugates described herein can be used for both human clinical medicine and veterinary applications. Thus, the host animal harboring the population of pathogenic cells and treated with the conjugates described herein can be human or, in the case of veterinary applications, can be a laboratory, agricultural, domestic, or wild animal. The conjugates described hereincan be applied to host animals including, but not limited to, humans, laboratory animals such rodents (e.g., mice, rats, hamsters, etc.), rabbits, monkeys, chimpanzees, domestic animals such as dogs, cats, and rabbits, agricultural animals such as cows, horses, pigs, sheep, goats, and wild animals in captivity such as bears, pandas, lions, tigers, leopards, elephants, zebras, giraffes, gorillas, dolphins, and whales.

The conjugate, compositions, methods, and uses described herein are useful for treating diseases caused at least in part by populations of pathogenic cells, which may cause a variety of pathologies in host animals. As used herein, the term “pathogenic cells” or “population of pathogenic cells” generally refers to cancer cells, infectious agents such as bacteria and viruses, bacteria- or virus-infected cells, inflammatory cells, activated macrophages capable of causing a disease state, and any other type of pathogenic cells that uniquely express, preferentially express, or overexpress cell surface receptors or cell surface anitgens that may be bound by or targeted by the conjugates described herein. Pathogenic cells can also include any cells causing a disease state for which treatment with the conjugates described herein results in reduction of the symptoms of the disease. For example, the pathogenic cells can be host cells that are pathogenic under some circumstances such as cells of the immune system that are responsible for graft versus host disease, but not pathogenic under other circumstances.

Thus, the population of pathogenic cells can be a cancer cell population that is tumorigenic, including benign tumors and malignant tumors, or it can be non-tumorigenic. The cancer cell population can arise spontaneously or by such processes as mutations present in the germline of the host animal or somatic mutations, or it can be chemically-, virally-, or radiation-induced. The conjugates described herein can be utilized to treat such cancers as carcinomas, sarcomas, lymphomas, Hodgekin's disease, melanomas, mesotheliomas, Burkitt's lymphoma, nasopharyngeal carcinomas, leukemias, and myelomas. The cancer cell population can include, but is not limited to, oral, thyroid, endocrine, skin, gastric, esophageal, laryngeal, pancreatic, colon, bladder, bone, ovarian, cervical, uterine, breast, testicular, prostate, rectal, kidney, liver, and lung cancers.

The disclosure includes all pharmaceutically acceptable isotopically-labelled conjugates, and their Drug(s) incorporated therein, wherein one or more atoms are replaced by atoms having the same atomic number, but an atomic mass or mass number different from the atomic mass or mass number which predominates in nature.

Examples of isotopes suitable for inclusion in the conjugates, and their Drug(s) incorporated therein, include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labelled conjugates, and their drug(s) incorporated therein, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, and ¹³N, can be useful in Positron Emission Topography (PET) studies for examining substrate receptor occupancy. Isotopically-labeled conjugates, and their drug(s) incorporated therein, can generally be prepared by conventional techniques known to those skilled in the art or by processes analogous to those described in the accompanying Examples using an appropriate isotopically-labeled reagents in place of the non-labeled reagent previously employed.

The conjugates and compositions described herein may be administered orally. Oral administration may involve swallowing, so that the conjugate or composition enters the gastrointestinal tract, or buccal or sublingual administration may be employed by which the conjugate or composition enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The conjugates and compositions described herein may also be used in fast-dissolving, fast-disintegrating dosage forms such as those described in Expert Opinion in Therapeutic Patents, 11 (6), 981-986, by Liang and Chen (2001). For tablet dosage forms, depending on dose, the conjugate may make up from 1 weight % to 80 weight % of the dosage form, more typically from 5 weight % to 60 weight % of the dosage form. In addition to the conjugates and compositions described herein, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colorants, flavoring agents, preservatives and taste-masking agents. Exemplary tablets contain up to about 80% drug, from about 10 weight % to 25 about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tableting. The final formulation may comprise one or more layers and may be coated or uncoated; it may even be encapsulated. The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a conjugate as described herein, a film-forming polymer, a binder, a solvent, a humectant, a plasticizer, a stabilizer or emulsifier, a viscosity-modifying agent and a solvent. Some components of the formulation may perform more than one function.

Solid formulations for oral administration may be formulated to be immediate and/or modified release. Modified release formulations include delayed-, sustained-, pulsed-, controlled-, targeted and programmed release. Suitable modified release formulations for the purposes of the disclosure are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

The conjugates described herein can also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous.

Suitable devices for parenteral administration include needle (including micro-needle) injectors, needle-free injectors and infusion techniques. Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art. The solubility of conjugates described herein used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus conjugates described herein can be formulated as a solid, semi-solid, or thixotropic liquid for administration as an implanted depot providing modified release of the active compound. Examples of such formulations include drug-coated stents and poly(lactic-coglycolic) acid (PGLA) microspheres. The conjugates described herein can also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J. Pharm Sci, 88 (10), 955-958 by Finnin and Morgan (October 1999). Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release. The conjugates described herein can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin. The pressurized container, pump, spray, atomizer, or nebulizer contains a solution or suspension of the conjugates(s) of the present disclosure comprising, for example, ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid. Prior to use in a dry powder or suspension formulation, the conjugate is micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying. Capsules (made, for example, from gelatin or hydroxypropylmethylcellulose), blisters and cartridges for use in an inhaler or insufflator may be formulated to contain a powder mix of the conjugate described herein, a suitable powder base such as lactose or starch and a performance modifier such as Iso-leucine, mannitol, or magnesium stearate.

The lactose may be anhydrous or in the form of the monohydrate, preferably the latter. Other suitable excipients include dextran, glucose, maltose, sorbitol, xylitol, fructose, sucrose and trehalose. A typical formulation may comprise a conjugate of the present disclosure, propylene glycol, sterile water, ethanol and sodium chloride. Alternative solvents which may be used instead of propylene glycol include glycerol and polyethylene glycol.

The conjugates described here can be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubilizer. Most commonly used for these purposes are alpha-, beta- and gamma-cyclodextrins, examples of which may be found in International Patent Applications Nos. WO 91/11172, WO 94/02518 and WO 98/55148.

Inasmuch as it may desirable to administer a combination of active compounds, for example, for the purpose of treating a particular disease or condition, it is within the scope of the present disclosure that two or more pharmaceutical compositions, at least one of which contains a conjugate as described herein, may conveniently be combined in the form of a kit suitable for co-administration of the compositions. Thus the kit of the present disclosure comprises two or more separate pharmaceutical compositions, at least one of which contains a conjugate as described herein, and means for separately retaining said compositions, such as a container, divided bottle, or divided foil packet. An example of such a kit is the familiar blister pack used for the packaging of tablets, capsules and the like. The kit of the present disclosure is particularly suitable for administering different dosage forms, for example parenteral, for administering the separate compositions at different dosage intervals, or for titrating the separate compositions against one another. To assist compliance, the kit typically comprises directions for administration and may be provided with a so-called memory aid.

### EXAMPLES

### Chemistry Examples

### Materials

Pteroic acid (Pte) and N¹⁰-trifluoroacetylpteroic acid were prepared according to Xu et al. (U.S. Pat. No. 8,044,200). EC0475 was prepared according to Vlahov et al. (United States Patent Application Publication No. US 2014/0080175 A1). EC1426, EC1427, and EC1428 were prepared according to Vlahov et al. (United States Patent Application Publication No. US 2014/0080175 A1). Des-glutamyl CB3717 (i.e., 5,8-dideazapteroic acid) and antifolate CB3717 may also be prepared according to known procedures (Jones et al. Eur. J. Cancer, 1981, 17(1), 11-9; Jones et al. J. Med. Chem., 1986, 29(6), 1114-8. Des-glutamyl AG147 and AG147 can be prepared according to known procedures (Wang et al. J. Med. Chem., 2013, 56, 8684-8695). Peptide synthesis reagents were purchased from Chem-Impex International (Wood Dale, Ill.), NovaBiochem (La Jolla, Calif.) and Bachem (San Carlos, Calif.). Boc-S-3-nitro-2-pyridinesulfenyl-L-cysteine (Boc-NPS-Cys) and α-t-butyl-γ-methyl L-Glu diester HCl salt were purchased from Chem-Impex International (Wood Dale, Ill.). All other common reagents were purchased from Sigma (St. Louis. Mo.) or other major suppliers.

### Synthesis of EC2133

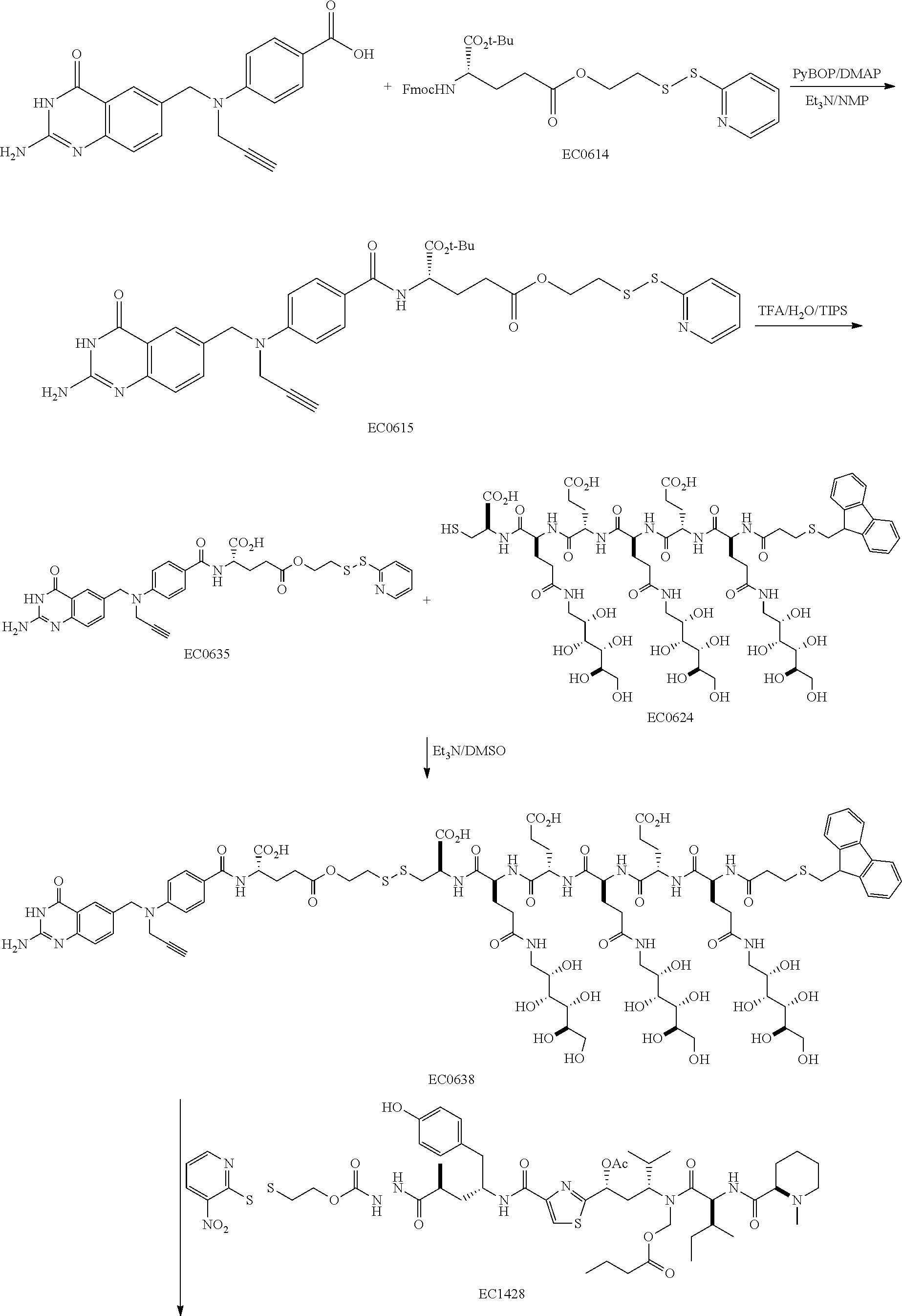

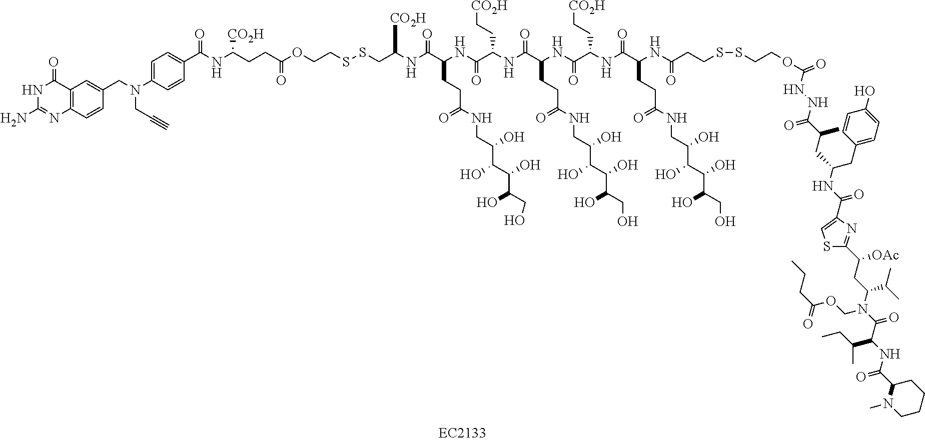 [SMILES: C#CCN(Cc1ccc2nc(N)[nH]c(=O)c2c1)c1ccc(C(=O)N[C@@H](CCC(=O)OCCSSC[C@H](NC(=O)[C@H](CCC(=O)NC[C@H](O)[C@@H](O)[C@H](O)[C@H](O)CO)NC(=O)[C@H](CCC(=O)O)NC(=O)[C@H](CCC(=O)NC[C@H](O)[C@@H](O)[C@H](O)[C@H](O)CO)NC(=O)[C@H](CCC(=O)O)NC(=O)[C@H](CCC(=O)NC[C@H](O)[C@@H](O)[C@H](O)[C@H](O)CO)NC(=O)CCSSCCOC(=O)NNC(=O)[C@@H](C)C[C@H](Cc2ccc(O)cc2)NC(=O)c2csc([C@@H](C[C@H](C(C)C)N(COC(=O)CCC)C(=O)[C@@H](NC(=O)[C@H]3CCCCN3C)C(C)CC)OC(C)=O)n2)C(=O)O)C(=O)O)cc1]

Reagents: Fmoc (tert-butyl)-L-glutamic acid (1.28 g, 3.00 mmol, 1 eq.), 2-(2-pyridyldithio)ethanol (684 mg, 3.00 mmol, 1 eq.), DMAP (806 mg, 6.60 mmol, 2.2 eq.) and HOBt (450 mg, 3.00 mmol, 1 eq.) were dissolved in dichloromethane (150 ml). The coupling reagent, DCC (680 mg, 3.3 mmol, 1.1 eq.) was then added to the solution, which was stirred at room temperature under argon overnight. The reaction mixture was filtered and the solvent evaporated. The desired product was dissolved in toluene. Dichloromethane was added and the organic solution washed with NaOAc (0.1M)/10% NaCl (pH 6), dried (MgSO₄) and filtered and the volatiles evaporated in vacuo to give a clear oil. The crude product was loaded onto a silica column and eluted with 50% EtOAc/petroleum ether to give the product EC0614 (1.5 g). ¹H NMR (CDCl₃) 8.47-8.44 (m, 1H), 7.46 (d, J=7.4 Hz, 2H), 7.68-7.58 (m, 4H), 7.39-7.26 (m, 4H), 7.10-7.05 (m, 1H), 5.42 (d, J=8.0 Hz, 1H), 4.40-4.26 (m, 4H), 4.22 (t, J=7.2 Hz, 1H), 3.03 (s, t, J=6.3 Hz, 2H), 2.50-2.30 (m, 2H), 2.28-2.18 (m, 1H), 2.02-1.85 (m, 1H), 1.48 (s, 9H). ¹³C NMR (CDCl₃) 172.7, 171.2, 159.8, 156.2, 149.9, 144.1, 143.9, 141.5, 137.26, 127.9, 127.3, 125.3, 121.1, 120.2, 120.1, 82.8, 67.3, 62.6, 53.9, 47.4, 37.4, 30.3, 28.2, 28.1

The Fmoc protected glutamic acid, EC0614 (614 mg) was dissolved in dimethylformamide (12 ml) before adding commercially available N¹⁰-propargyl-5,8-dideazapteroic acid (350 mg), PyBOP (510 mg), HOBt (150 mg) and DMAP (135 mg). The reaction mixture was then stirred for 5 mins. Triethylamine (0.3 ml) was added and the reaction mixture was left to stir overnight. The reaction mixture was added to a NaOAc (0.1M)/10% NaCl (pH=6) solution, which was centrifuged. The resulting crude product (601 mg) was purified by HPLC using ACN/10 mM NH₄OAc buffer, pH 5.2 as an eluent to afford the product, EC0615 (360 mg). ¹H NMR (DMSO-d6) 10.92 (br, 1H, lactam NH), 8.42 (dd, J=4.8, 1.0 Hz, 1H), 8.23 (d, J=8.7 Hz, 1H), 7.82-7.70 (m, 5H), 7.47 (dd, J=8.3, 2.1 Hz, 1H), 7.20 (m, 1H), 7.13 (d, J=8.7 Hz, 1H), 6.83 (d, J=8.8 Hz, 2H), 6.28 (br, 2H), 4.64 (s, 2H), 4.32-4.19 (m, 5H), 3.19 (t, J=2.2 Hz, 1H), 3.07 (t, J=6.0 Hz, 2H), 2.37 (t, J=7.5 Hz, 2H), 2.10-1.83 (m, 2H), 1.37 (s, 9H). ¹³C NMR (DMSO-D6) 172.8, 171.9, 167.1, 159.6, 150.6, 150.3, 138.4, 133.8, 132.0, 129.9, 124.7, 122.8, 121.9, 119.9, 117.7, 112.9, 81.2, 80.8, 75.4, 62.4, 54.4, 53.0, 37.5, 35.9, 30.8, 28.4, 26.6.

EC0615 (20 mg) was dissolved in a 2.5% TFA/2.5% TIPS/2.5% H₂O cleavage solution (1 ml) at room temperature. Reaction progress was monitored by LC/MS. After reaching completion, the reaction mixture was precipitated into cold, diethyl ether. The resulting precipitate was centrifuged and the solvent decanted before washing the solid portion with diethyl ether and centrifuging the suspension again. The solid portions were collected and, after air drying, yielded the desired product EC0635 (18 mg).

### Synthesis of EC0624

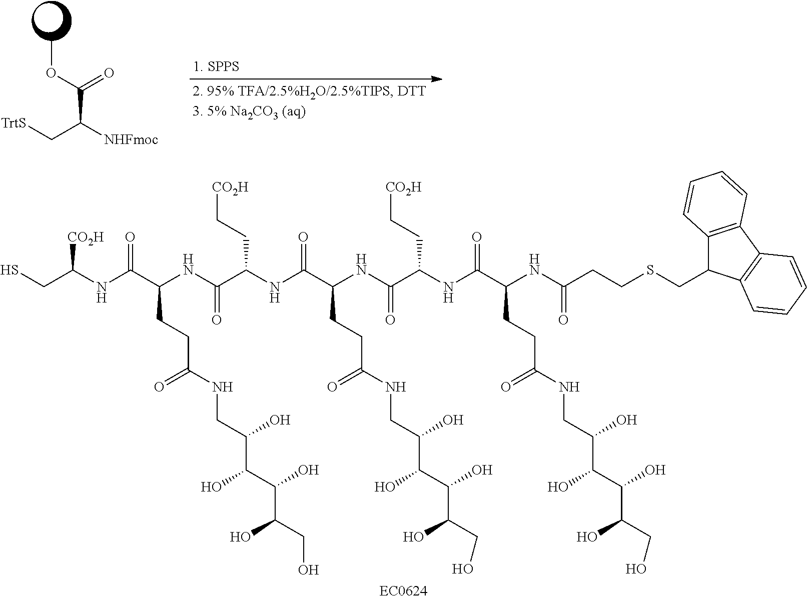

The peptidic spacer EC0624 was synthesized using Fmoc-standard solid phase peptide synthesis (Fmoc-SPPS) from H-Cys(trityl)-2-chlorotrityl resin (6.56 g, 4.00 mmol, 1 eq., loading 0.61 mmol/g) as follows:

1) a. EC0475 (4.90 g, 8.00 mmol, 2 eq.), PyBOP (6.24 g, 12.0 mmol, 3 eq.), DIPEA (2.07 g, 16.0 mmol, 4 eq.); b. 20% Piperidine/DMF;
2) a. Fmoc-L-glutamic acid 5-tert-butyl ester (3.40 g, 8.00 mmol, 2 eq.), PyBOP (6.24 g, 12.0 mmol, 3 eq.), DIPEA (2.07 g, 16.0 mmol, 4 eq.); b. 20% Piperidine/DMF;
3) a. EC0475 (4.90 g, 8.00 mmol, 2 eq.), PyBOP (6.24 g, 12.0 mmol, 3 eq.), DIPEA (2.07 g, 16.0 mmol, 4 eq.); b. 20% Piperidine/DMF;
4) a. Fmoc-L-glutamic acid 5-tert-butyl ester (3.40 g, 8.00 mmol, 2 eq.), PyBOP (6.24 g, 12.0 mmol, 3 eq.), DIPEA (2.07 g, 16.0 mmol, 4 eq.); b. 20% Piperidine/DMF;
5) a. EC0475 (4.90 g, 8.00 mmol, 2 eq.), PyBOP (6.24 g, 12.0 mmol, 3 eq.), DIPEA (2.07 g, 16.0 mmol, 4 eq.); b. 20% Piperidine/DMF;
6) a. Fluorenylmethyl thiopropanoic acid (FMTPA) (4.16 g, 8.00 mmol, 2 eq.), PyBOP (6.24 g, 12.0 mmol, 3 eq.), DIPEA (2.07 g, 16.0 mmol, 4 eq.).

The resin was washed consecutively with DMF (3×20 ml), IPA (3×20 ml) and DMF (3×20 ml). After drying in vacuum for 18 h, 6.56 g of the loaded resin was collected. Treatment of the loaded resin (3.48 g, 2.13 mmol, 1 eq.) with a 92.5% TFA/2.5% TIPS/5% H₂O cleavage solution (150 ml) and dithiothreitol (1.31 g, 8.52 mmol, 4 eq.) for 1 h resulted in resin cleavage, Trityl removal and partial removal of the tert-butyl ester and acetamide protecting groups. Most of the cleavage solution (130 ml) was removed under reduced pressure and the crude product precipitated with ether. The solid portions were then centrifuged down and the resulting white solid dissolved in a 5% Na₂CO₃ aqueous solution purged with argon. After 0.5 h of argon bubbling, the solution was purified by reverse-phase chromatography using 0-80% ACN/0.1% TFA as the eluent. Collection and lyophilysis of fractions containing the desired product afforded EC0624 as a white powder (509 mg, 16%). ¹H NMR (500 MHz, DMSO-d₆): δH=8.17 (1H, d, J=8.5 Hz, NH); 8.09 (2H, t, J=8.5 Hz, 2×NH); 8.03 (2H, t, J=8.0 Hz, 2×NH); 7.98 (1H, d, J=8.0 Hz, NH); 7.83 (2H, d, J=8.0 Hz, 2×ArH); 7.72 (2H, d, J=7.0 Hz, 2×ArH); 7.68-7.70 (2H, m, 2×NH); 7.65 (1H, t, J=5.5 Hz, NH); 7.37 (2H, t, J=7.5 Hz, 2×ArH); 7.30 (2H, t, J=7.0 Hz, 2×ArH); 4.40 (1H, m); 4.14-4.25 (6H, m); 3.54-3.62 (8H, m); 3.44-3.47 (4H, m); 3.45-3.38 (6H, m); 3.23-3.27 (4H, m); 3.12 (2H, d, J=6.5 Hz); 2.98-3.02 (2H, m); 2.72-2.87 (2H, m); 2.66 (2H, t, J=7.0 Hz); 2.40-2.41 (2H, m); 2.20-2.25 (3H, m); 2.09-2.16 (4H, m); 1.86-1.89 (4H); 1.70-1.74 (4H). MS (ESI): m/z 1523.92 amu (M+H), 761.81 amu (M+2H); calc. for C₆₃H₉₅N₉O₃₀S₂: 1523.60 amu, 762.30 amu.

EC0635 (16 mg, 0.025 mmol) was dissolved in dimethyl sulfoxide (0.8 ml) and EC0624 (37 mg, 0.024 mmol) added. The solution was purged with argon and triethylamine (17 μL, 0.122 mmol, 5 eq.) added. Reaction progress was monitored by LC/MS. When complete, the reaction mixture was diluted with a cold 0.1% TFA aqueous solution. Purification by HPLC using ACN/0.1% TFA (aq. solution) as the eluent afforded the desired product EC0638 (42 mg).

EC1428 was prepared as described by Vlahov et al. in United States Patent Application Publication No. US 2014/0080175 A1 (see compound 2 described therein), the disclosure of which is incorporated by reference for the preparation of EC1428. EC0638 (11 mg, 0.0053 mmol) and EC1428 (9.0 mg, 0.0082 mmol, 1.5 eq.) were dissolved in dimethyl sulfoxide (0.6 ml) and the solution purged with argon. Triethylamine (7.5 μL, 10 eq.) was added followed by 50 μL DBU/DMSO solution (40 μL of DBU in 460 μL of DMSO, 10 eq.). Reaction progress was monitored by LC/MS. When complete, the reaction mixture was diluted with cold water and purified by HPLC using ACN/50 mM NH₄HCO₃ (pH 7). Lyophilization yielded the desired product EC2133 (5.5 mg). Selected 1H-NMR (D2O, 500 MHz) δ(ppm): 7.98 (s, 1H), 7.74 (s, 1H), 7.56 (d, J=8.5 Hz, 2H), 7.38 (d, 1H), 7.06 (d, 1H), 6.86 (d, 2H), 6.72 (d, 2H), 6.55 (d, 2H), 5.98 (m, 1H), 5.62 (d, J=11 Hz, 1H), 5.11 (d, J=11 Hz, 1H).

### Synthesis of EC1822

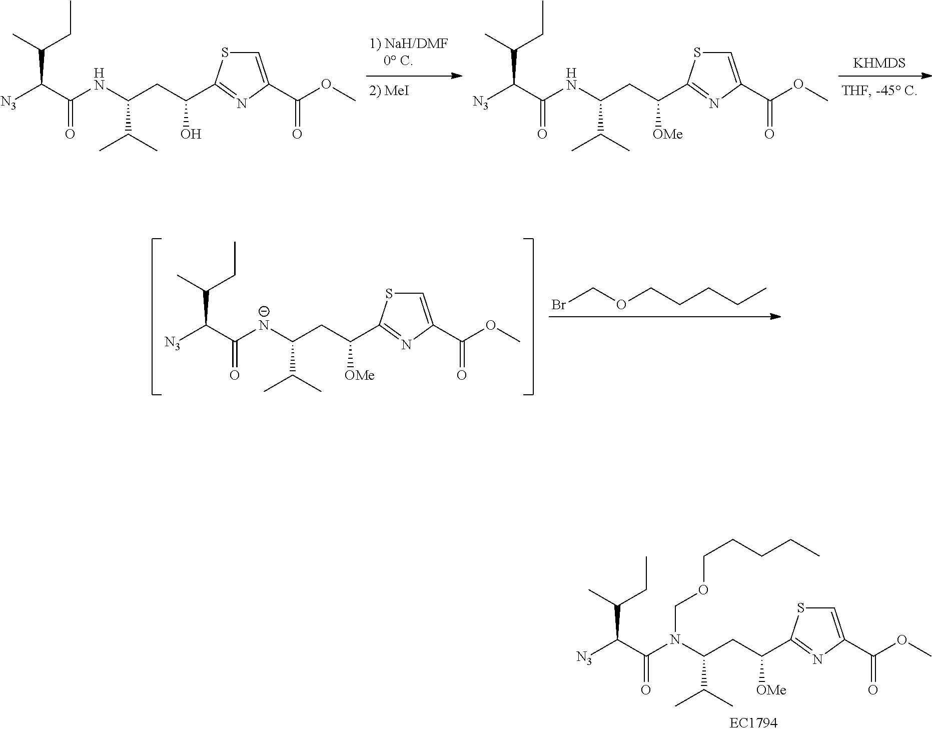

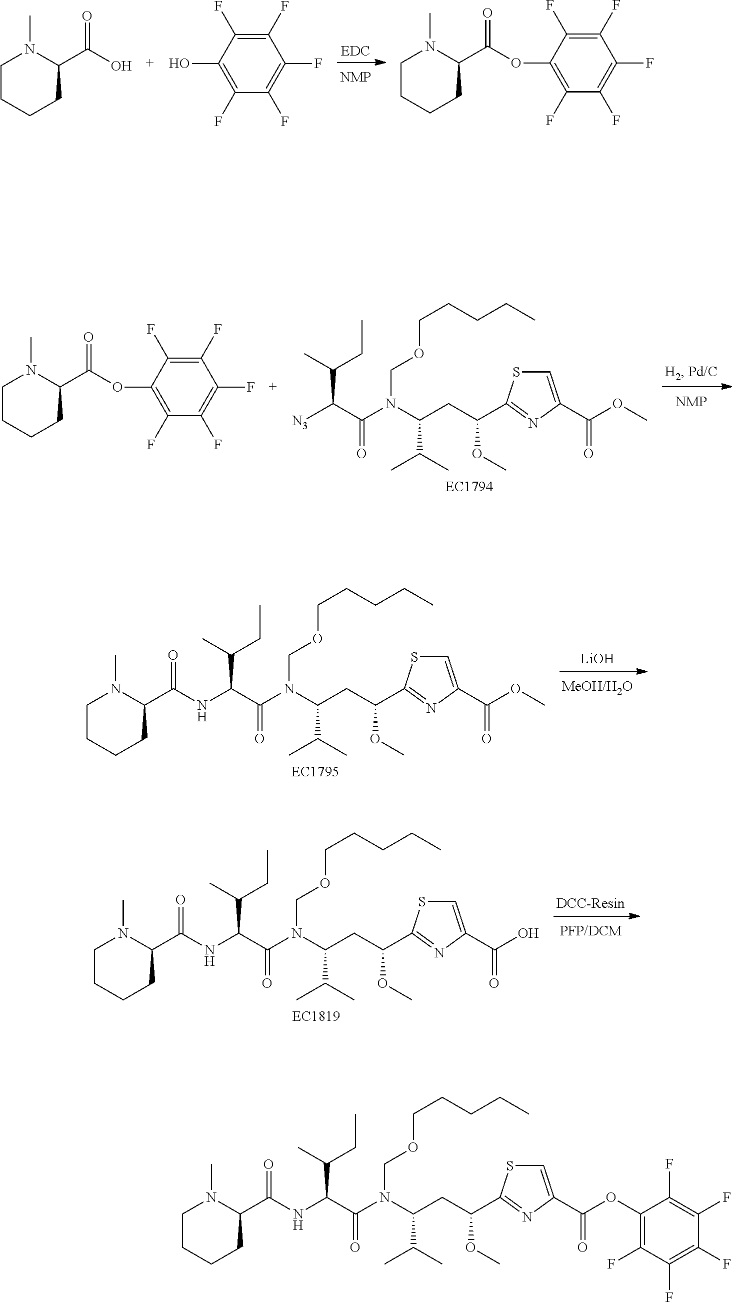

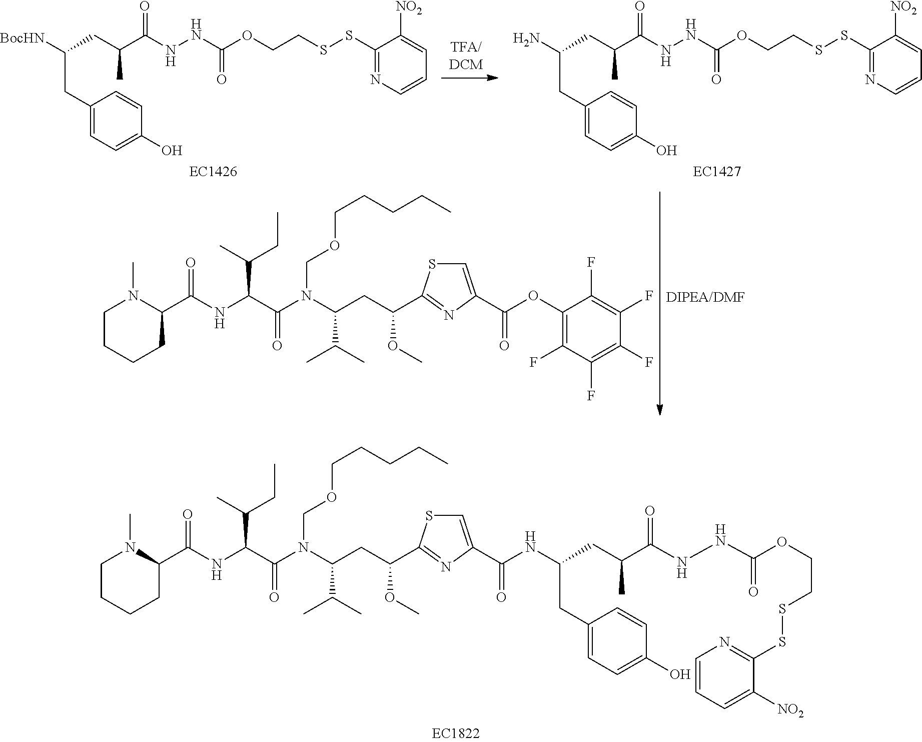 [SMILES: C[C@@H](C[C@H](Cc1ccc(O)cc1)NC(=O)OC(C)(C)C)C(=O)NNC(=O)OCCSSc1ncccc1[N+](=O)[O-] ; C[C@@H](C[C@@H](N)Cc1ccc(O)cc1)C(=O)NNC(=O)OCCSSc1ncccc1[N+](=O)[O-] ; CCCCCOCN(C(=O)[C@@H](NC(=O)[C@H]1CCCCN1C)C(C)CC)[C@H](C[C@@H](OC)c1nc(C(=O)Oc2c(F)c(F)c(F)c(F)c2F)cs1)C(C)C ; CCCCCOCN(C(=O)[C@@H](NC(=O)[C@H]1CCCCN1C)C(C)CC)[C@H](C[C@@H](OC)c1nc(C(=O)N[C@@H](Cc2ccc(O)cc2)C[C@H](C)C(=O)NNC(=O)OCCSSc2ncccc2[N+](=O)[O-])cs1)C(C)C]

1.2 g of dipeptide (3 mmole) was dissolved in 3 mL of DMF and cooled to 0° C. To the solution, 120 mg of NaH (60% in mineral oil, 3 mmole) was added. After 30 min. reaction, 200 μL of MeI (1.08 equiv.) was added. After 2 hr, LC/MS showed majority of the starting material was converted. The reaction was worked up by extraction between EtOAc and H₂O. The organic layer was washed with H₂O, brine, and dried over Na₂SO₄. The solvent was removed under reduced pressure to give oily residue. Purification with Combiflash using EtOAc/Petroleum ether gave 0.8 g (65%) of desired methyl ether product.

0.8 g of dipeptide methyl ether (1.95 mmole) was dissolved in 8 mL of anhydrous THF (inhibitor-free). The solution was cooled to −45° C. with dry ice/acetonitrile bath. After 15 min, 4.1 mL of KHMDS (0.5 M in toluene, 2.05 mmole, 1.05 equiv.) was added dropwise. The resulted reaction mixture was stirred at −45° C. for 15 min. 420 μL of bromomethyl pentyl ether was added. After 30 min, LC/MS showed no dipeptide methyl ether left. The reaction was worked up by extraction between 10% NaCl/1% NaHCO₃ aqueous solution and EtOAc. The organic layer was washed with 10% NaCl/1% NaHCO₃ aqueous solution twice, then with brine, dried over Na₂SO₄. The solvent was removed under reduced pressure. Purification on Combiflash with MeOH/DCM gave 210 mg (21%) of the desired product EC1794. LCMS (ESI) [M+H]⁺ 512.39. ¹H NMR (CD3OD): 7.95 (s, 1H), 4.75 (d, J=10.3 Hz, 1H), 4.55 (d, J=10.3 Hz, 1H), 4.51 (dd, J=10.3, 2.4 Hz, 1H), 3.90 (s, 3H), 3.76 (d, J=9.3 Hz, 1H), 3.51-3.48 (m, 1H), 3.44-3.40 (m, 1H), 3.35 (s, 3H), 2.20-2.18 (m, 2H), 2.01 (m, br, 1H), 1.83-1.70 (m, 2H), 1.68-1.52 (m, 2H), 1.38-1.24 (m, 5H), 1.01-0.96 (m, 9H), 0.88 (d, J=6.8 Hz, 3H), 0.85 (t, br, J=6.8 Hz, 3H).

60 mg of MEP (0.42 mmole, 1.4 equiv compared to EC1794) was suspended in 1.0 mL NMP. To the suspension, 83 mg of pentafluorophenol (0.45 mmole, 1.5 equiv.) and 86 mg of EDC (0.45 mmole, 1.5 equiv) were added. The reaction mixture was stirred overnight at room temperature. The reaction mixture was transferred into a hydrogenation vessel with 151 mg of EC1794 in 1.0 mL NMP. To the resulting mixture, 25 mg of 10% Pd/C (dry, 0.05 equiv) was added. The hydrogenation vessel was pumped/filled with H₂ three times. Hydrogenation was carried out with 35 PSI H₂ for 3 hr. LC/MS showed no EC1794 left. The reaction mixture was passed through celite pad and washed with EtOAC. The organic solution was extracted with EtOAc and 10% NaCl/1% NaHCO₃ aqueous solution. The organic layer was washed with brine, and dried over Na₂SO₄. The solvent was removed under reduced pressure after filtering off Na₂SO₄. Purification on Combiflash with MeOH/DCM gave 68 mg (38%) of EC1795. LCMS (ESI) [M+H]⁺ 611.39. ¹H NMR (500 MHz, CD3OD): 8.39 (s, 1H), 5.35 (d, J=9.8 Hz, 1H), 4.70 (d, J=9.3 Hz, 1H), 4.59 (d, J=11.3 Hz, 1H), 4.42 (d, J=9.8 Hz, 1H), 3.90 (s, 3H), 3.51 (m, 2H), 3.33 (s, 3H), 2.96 (dd, br, J=12.7 Hz, 1H), 2.67 (dd, br, J=10.8 Hz, 1H), 2.22 (s, 3H), 2.18-1.98 (m, 5H), 1.79 (m, 3H), 1.70-1.50 (m, 7H), 1.40-1.20 (m, 6H), 1.01 (d, J=6.3 Hz, 3H), 0.98 (d, J=6.3 Hz, 3H), 0.92 (t, J=6.8 Hz, 3H), 0.84 (t, J=6.8 Hz, 3H), 0.76 (d, J=6.3 Hz, 3H).

¹³C NMR (125 MHz, CD3OD): 175.07, 174.24, 173.43, 161.73, 146.32, 128.20, 77.48, 68.89, 67.27, 56.76, 55.16, 53.66, 51.24, 43.15, 37.15, 36.37, 31.07, 30.04, 28.90, 28.28, 24.58, 24.33, 22.73, 22.06, 19.26, 18.89, 15.16, 12.96, 9.37.

24 mg of EC1795 (0.039 mmole) was dissolved in 0.8 mL of MeOH and cooled to 0° C. 7.3 mg (0.17 mmole, 4.4 eq) of LiOH monohydrate was dissolved in 0.2 mL H₂O and was added to EC1795 solution. The reaction mixture was warmed up to room temperature. After 1 hr, LC/MS showed completed conversion. The solvent was removed under vacuum. The residue of EC1819 was dried under high vacuum and used without further purification. LCMS (ESI) [M−H]⁻ 595.68. ¹H NMR (500 MHz, CD3OD): 7.95 (s, 1H), 5.28 (d, J=10.3 Hz, 1H), 4.68 (d, J=8.8 Hz, 1H), 4.55 (d, J=12.2 Hz, 1H), 4.46 (d, J=10.3 Hz, 1H), 3.50 (t, J=6.8 Hz, 2H), 3.30 (s, 3H), 3.02 (br, 1H), 2.27 (s, br, 3H), 2.23-2.10 (m, 2H), 2.07-1.94 (m, 2H), 1.88-1.74 (m, 3H), 1.70-1.46 (m, 6H), 1.40-1.27 (m, 6H), 1.21 (m, 1H), 1.00 (d, J=6.8 Hz, 3H), 0.98 (d, J=6.4 Hz, 3H), 0.92 (t, J=7.4 Hz, 3H), 0.87 (t, J=7.4 Hz, 3H), 0.80 (br, 3H).

20 mg of EC1819 (0.034 mmole) was mixed with 72 mg of DCC-resin (5 equiv) and 12 mg of PFP (2 equiv) in 1.0 mL anhydrous DCM. The reaction mixture was stirred at room temperature overnight. LC/MS showed complete conversion. The resin was filtered off and washed with DCM. The resulted solution was concentrated under reduced pressure and dried over high vacuum for 30 min.

20 mg of EC1426 was dissolved in 0.3 mL of TFA/DCM (1:1). After 30 min, LC/MS showed complete conversion. The solvent was removed under reduced pressure and the residue was dried under high vacuum overnight and used without further purification.

The EC1819-PFP ester was dissolved in 0.5 mL DMF. To the solution, 123 μL of DIPEA was added. EC1427 was dissolved in 0.2 mL of DMF. These two solutions were mixed and stirred at room temperature for 2 hr. LC/MS showed complete consumption of EC1819-PFP ester. The reaction mixture was extracted between EtOAc/brine. The organic layer was dried over Na₂SO₄. The solvent was removed under reduced pressure after filtering off Na₂SO₄. Purification on Combiflash with MeOH/DCM gave 13.7 mg (38%) of EC1822. LCMS (ESI) [M+H]⁺ 1075.11. ¹H NMR (500 MHz, CD3OD): 8.88 (s, br, 1H), 8.56 (d, J=7.8 Hz, 1H), 8.12 (s, 1H), 7.45 (t, J=8.3, 4.4 Hz, 1H), 7.02 (m, 2H), 6.65 (d, J=8.3 Hz, 2H), 5.40 (d, J=10.3 Hz, 1H), 4.68 (d, J=9.3 Hz, 1H), 4.56 (d, J=11.2 Hz, 1H), 4.40 (d, J=9.8 Hz, 1H), 4.36 (t, J=6.4 Hz, 2H), 3.50-3.45 (m, 1H), 3.42-3.38 (m, 1H), 3.36 (s, 3H), 3.30 (s, 3H), 3.16-3.09 (m, 3H), 2.88 (dd, br, 1H), 2.86-2.72 (m, 1H), 2.69 (dd, br, 1H), 2.45 (m, 1H), 2.22 (s, 3H), 2.18-2.10 (m, 2H), 2.07-1.94 (m, 3H), 1.84 (m, 1H), 1.82-1.74 (m, 3H), 1.70-1.46 (m, 7H), 1.40-1.20 (m, 7H), 1.12 (d, J=6.8 Hz, 3H), 1.00 (dd, J=6.3 Hz, 6H), 0.92 (t, J=7.4 Hz, 3H), 0.83 (t, J=6.8 Hz, 3H), 0.78 (d, J=6.8 Hz, 3H). ¹³C NMR (125 MHz, CD3OD): 177.04, 175.06, 173.28, 161.87, 156.70, 155.83, 155.70, 153.70, 149.42, 142.87, 133.68, 130.23, 123.72, 114.82, 77.51, 68.83, 67.06, 63.43, 56.98, 55.17, 53.71, 49.21 45.94, 43.11, 39.65, 38.96, 37.36, 36.75, 36.73, 36.30, 35.49, 31.13, 30.01, 29.06, 28.36, 25.97, 25.90, 24.53, 24.37, 22.70, 22.07, 19.33, 18.94, 17.23, 15.12, 13.02, 9.39.

### Synthesis of EC2150

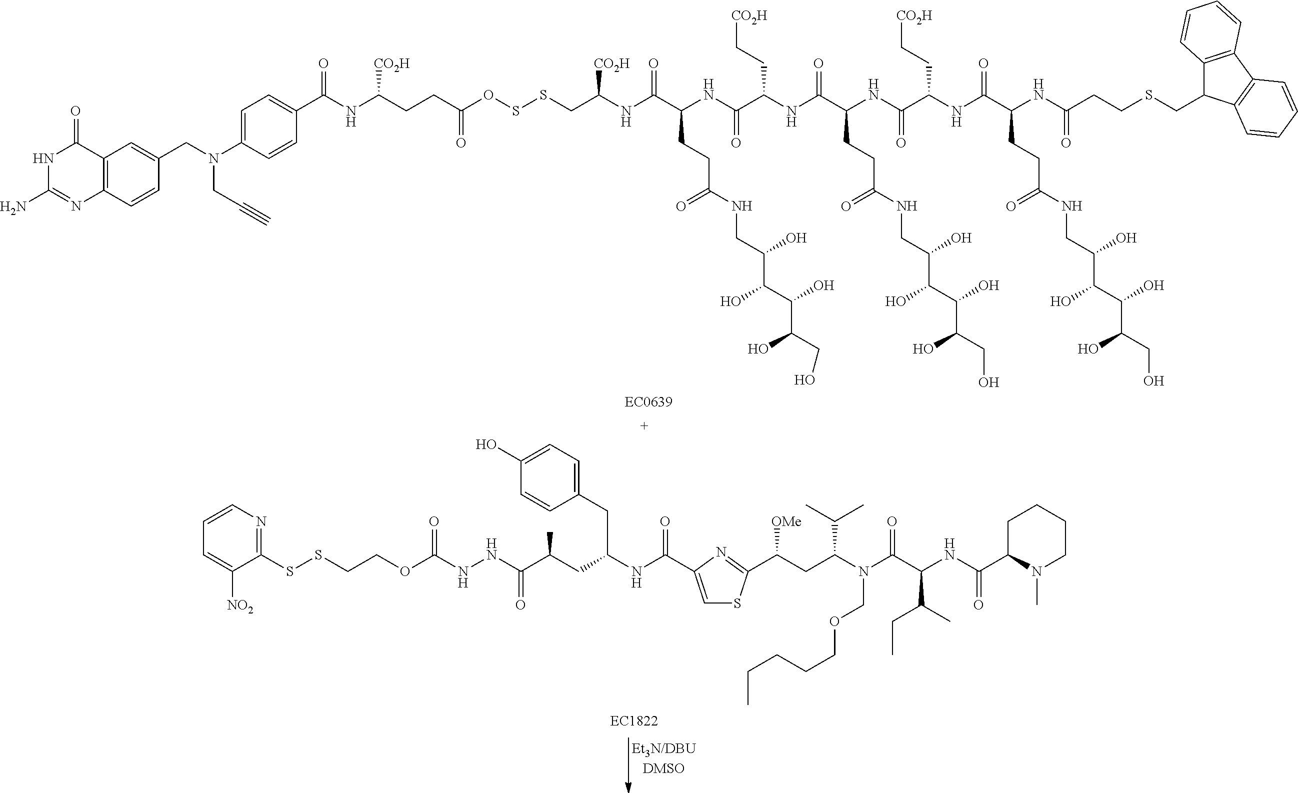 [SMILES: C#CCN(Cc1ccc2nc(N)[nH]c(=O)c2c1)c1ccc(C(=O)N[C@@H](CCC(=O)OSSC[C@H](NC(=O)[C@H](CCC(=O)NC[C@H](O)[C@@H](O)[C@H](O)[C@H](O)CO)NC(=O)[C@H](CCC(=O)O)NC(=O)[C@H](CCC(=O)NC[C@H](O)[C@@H](O)[C@H](O)[C@H](O)CO)NC(=O)[C@H](CCC(=O)O)NC(=O)[C@H](CCC(=O)NC[C@H](O)[C@@H](O)[C@H](O)[C@H](O)CO)NC(=O)CCSCC2c3ccccc3-c3ccccc32)C(=O)O)C(=O)O)cc1 ; CCCCCOCN(C(=O)[C@@H](NC(=O)[C@H]1CCCCN1C)C(C)CC)[C@H](C[C@@H](OC)c1nc(C(=O)N[C@@H](Cc2ccc(O)cc2)C[C@H](C)C(=O)NNC(=O)OCCSSc2ncccc2[N+](=O)[O-])cs1)C(C)C]

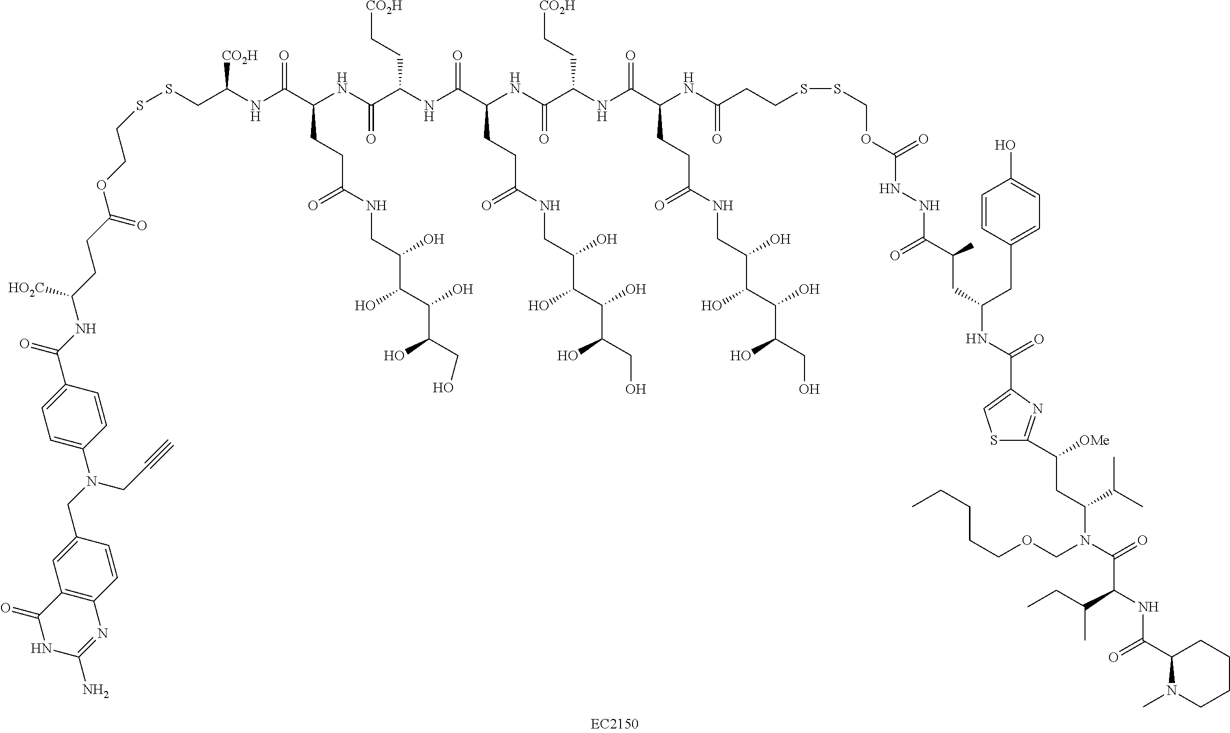 [SMILES: C#CCN(Cc1ccc2nc(N)[nH]c(=O)c2c1)c1ccc(C(=O)N[C@@H](CCC(=O)OCCSSC[C@H](NC(=O)[C@H](CCC(=O)NC[C@H](O)[C@@H](O)[C@H](O)[C@H](O)CO)NC(=O)[C@H](CCC(=O)O)NC(=O)[C@H](CCC(=O)NC[C@H](O)[C@@H](O)[C@H](O)[C@H](O)CO)NC(=O)[C@H](CCC(=O)O)NC(=O)[C@H](CCC(=O)NC[C@H](O)[C@@H](O)[C@H](O)[C@H](O)CO)NC(=O)CCSSCOC(=O)NNC(=O)[C@@H](C)C[C@H](Cc2ccc(O)cc2)NC(=O)c2csc([C@@H](C[C@H](C(C)C)N(COCCCCC)C(=O)[C@@H](NC(=O)[C@H]3CCCCN3C)C(C)CC)OC)n2)C(=O)O)C(=O)O)cc1]

EC0638 (13 mg, 0.0063 mmol) and EC1822 (8.4 mg, 0.0078 mmol, 1.2 eq.) were dissolved in dimethylsulfoxide (0.6 mL). The resulting solution was purged with argon and triethylamine (8.8 μL, 10 eq.) added, followed by 50 μL DBU/DMSO solution (48 μL DBU in 452 μL DMSO, 5 eq.). Reaction progress was monitored by LC/MS. When complete, the reaction mixture was diluted with cold water and purified by HPLC with ACN/50 mM NH₄HCO₃ (pH 7). After lyophilization, the desired product EC2150 (7.3 mg) was obtained. Selected 1H-NMR (D2O, 500 MHz) δ(ppm): 8.09 (s, 1H), 7.84 (s, 1H), 7.66 (d, J=8.5 Hz, 2H), 7.48 (d, J=8.5 Hz, 1H), 7.18 (d, J=8.5 Hz, 1H), 6.96 (d, J=8 Hz, 2H), 6.78 (d, J=8.5 Hz, 2H), 6.67 (d, J=6.5 Hz 2H), 5.15 (d, J=8.5 Hz, 1H).

### Synthesis of EC2412

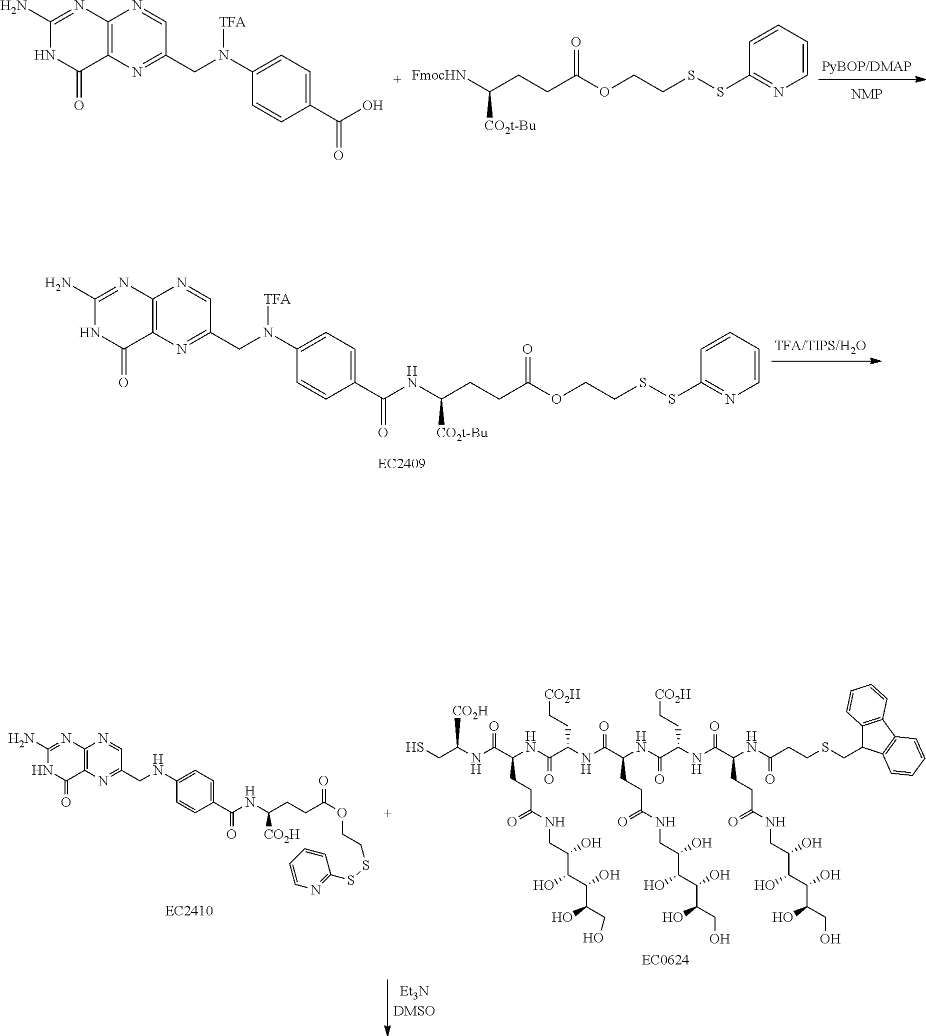

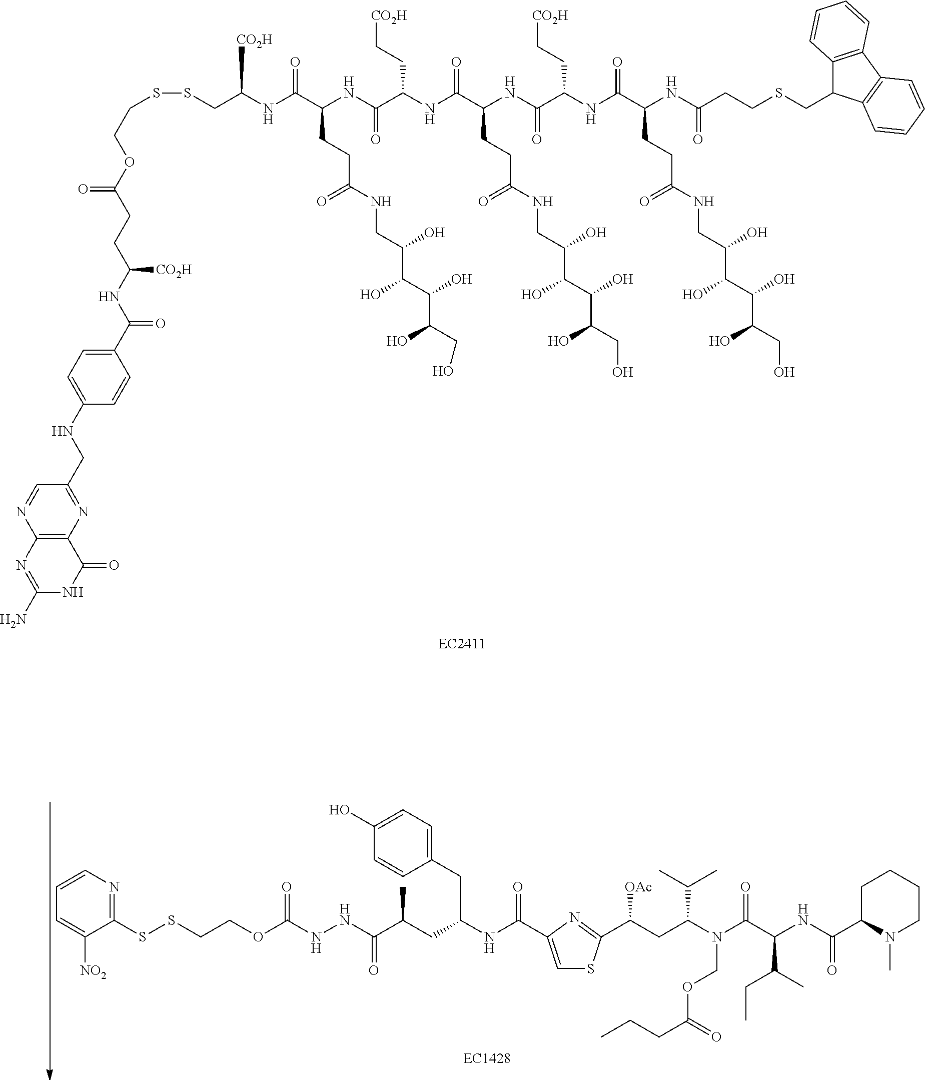

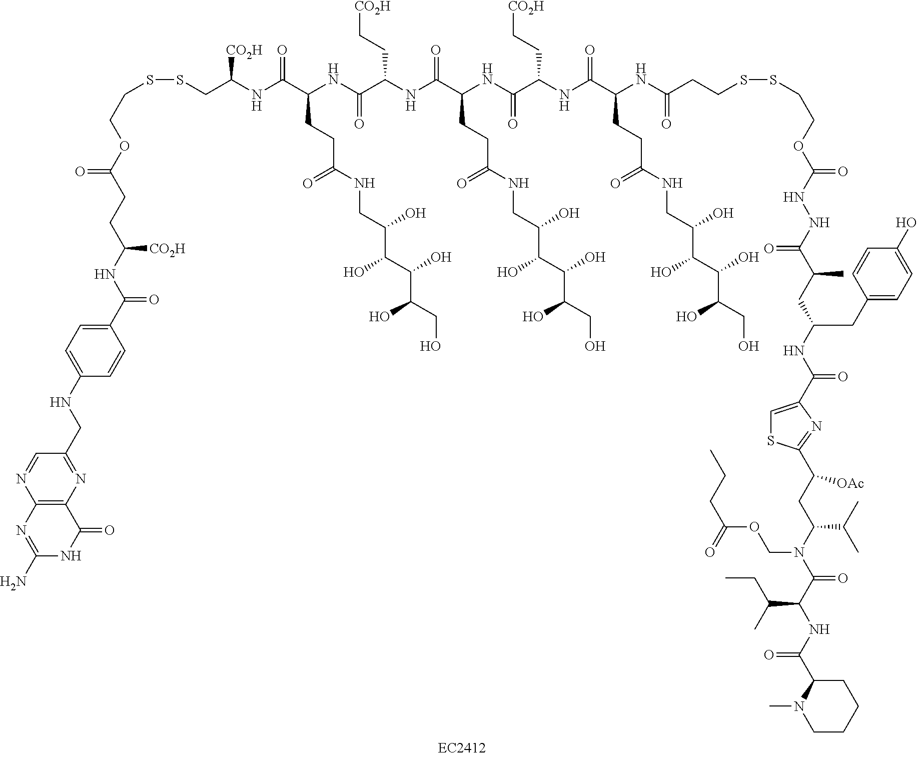

N¹⁰-trifluoroacetylpteroic acid was prepared as described by Xu et al. in U.S. Pat. No. 8,044,200). N¹⁰-TFA pteroic acid (81 mg, 0.2 mmol), EC0614 (120 mg, 0.2 mmol, 1 eq.), PyBOP (125 mg, 0.24 mmol, 1.2 eq.) and DMAP (122 mg, 5 eq.) were dissolved in NMP (2 mL). The reaction mixture was stirred at room temperature and reaction progress monitored by LC/MS. After 2 h, the reaction mixture was purified on a 12 g, C18 column with medium pressure using ACN/H₂O as an eluent. The desired product EC2409 (92 mg) was obtained after lyophilization.

EC2409 (20 mg, 0.06 mmol) was dissolved in 1 mL of cleavage solution (95% TFA/2.5% TIPS/2.5% H₂O) at room temperature. Reaction progress was monitored by LC/MS. After completion, the reaction mixture was precipitated with cold diethyl ether. The precipitate was centrifuged and the solvent decanted. The solid portion was washed with diethyl ether again and then air-dried for 1 h. After an additional 2 h of drying under high vacuum, the desired product EC2410 (13 mg) was obtained.

EC2410 (13 mg, 0.021 mmol) and EC0624 (32 mg, 0.021 mmol, 1 eq.) were dissolved in dimethyl sulfoxide (1 mL). The solution was purged with argon for 10 mins and triethylamine (29 μL, 10 eq.) added. Reaction progress was monitored by LC/MS. After completion, the reaction mixture was diluted with cold H₂O, and purified by HPLC with ACN/0.1% TFA. The desired product EC2411 (20 mg) was obtained after lyophilization.

EC2411 (13.3 mg, 0.0066 mmol) and EC1428 (9.3 mg, 0.0084 mmol, 1.28 eq.) were dissolved in dimethyl sulfoxide (0.8 mL). The solution was purged with argon for 15 mins and 200 μL of a DBU/DMSO solution (50 μL DBU in 950 μL DMSO, 10 eq.) was added. The reaction was monitored by LC/MS and, additional DBU/DMSO added as needed. After the reaction was completed, the reaction mixture was purified by HPLC with ACN/50 mM NH₄HCO₃ (pH 7) buffer. The desired product, EC2412 (8 mg, 44%) was obtained following lyophilization. Selected ¹H-NMR (D2O, 500 MHz) δ(ppm): 8.54 ((s, 1H), 7.97 (s, 1H), 7.48 (d, J=8 Hz, 1H), 6.83 (d, J=7.5 Hz, 2H), 6.55 (d, J=8 Hz, 2H), 6.53 (d, 2H), 5.97 (br, 1H), 5.62 (d, J=10 Hz, 1H), 5.13 (d, J=10 Hz, 1H). MS (ESI): m/z 1396.55 [M+2H]^{2±}.

### Comparative Examples

### Comparative Example 1 (CB3717, 5,8-dideazapteroic Acid)

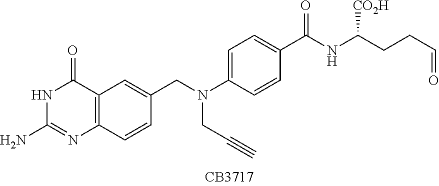 [SMILES: C#CCN(Cc1ccc2nc(N)[nH]c(=O)c2c1)c1ccc(C(=O)N[C@@H](CCC=O)C(=O)O)cc1]

### Comparative Example 2 (EC0347, Tubulysin B Hydrazide)

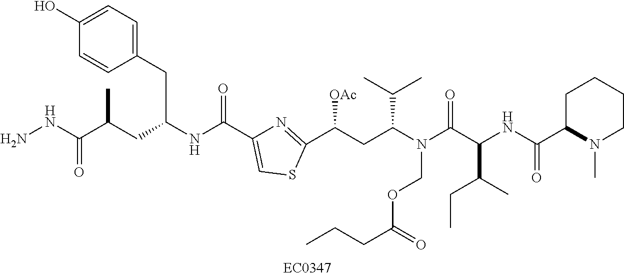 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NN)cs1)C(C)C]

### Comparative Example 3 (ECO284)

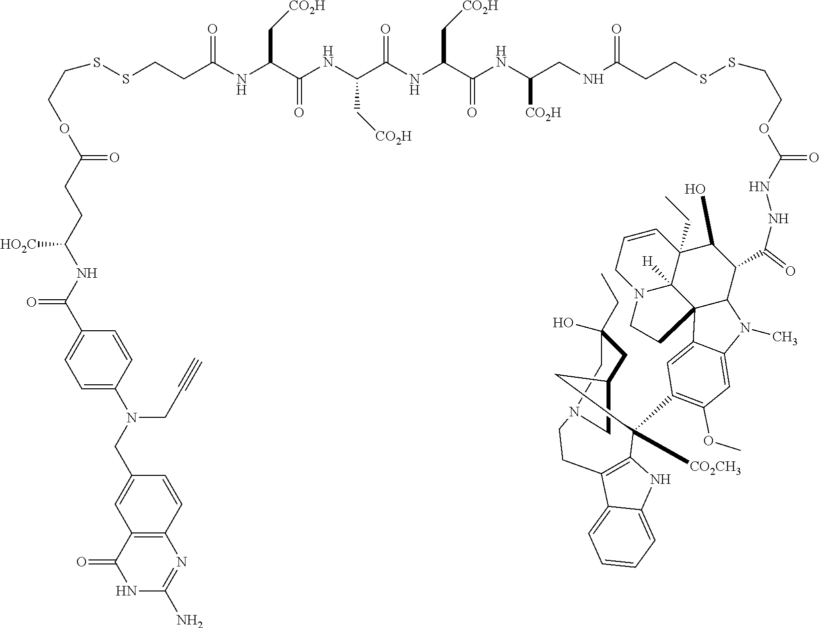

### Biological Examples

### In Vitro Activity in Kb Cells

Cells were seeded in 24-well Falcon plates and allowed to form nearly confluent monolayers overnight. After one rinse with 1 mL of fresh FFRPMI/HIFCS, each well received 1 mL, of medium containing increasing concentrations of test agent (3 wells per sample). Cells were pulsed with targeted agents for 2 hr at 37° C., rinsed 4 times with 0.5 mL of medium, and then chased in 1 mL of fresh medium up to 70 hr. Cells were treated with non-targeted agent EC0347 for a continuous 72 h. Spent medium was aspirated from all wells and replaced with fresh medium containing 5 μCi/mL ³H-thymidine. After a further 2 hr 37° C. incubation, cells were washed 3 times with 0.5 mL of PBS and then treated with 0.5 mL of ice-cold 5 trichloroacetic acid per well. After 15 min, the trichloroacetic acid was aspirated and the cell material solubilized by the addition of 0.5 mL of 0.25 N sodium hydroxide for 15 min. Four hundred and fifty microliters of each solubilized sample were transferred to scintillation vials containing 3 mL of Ecolume scintillation cocktail and then counted in a liquid scintillation counter. Final tabulated results were expressed as the percentage of ³H-thymidine incorporation relative to untreated controls and IC50 values calculated using Graphpad Prism software. Results are shown in the table below.

| | | | Cellular | |
|---|---|---|---|---|
| Test Article | Ligand | Drug | Target | IC₅₀ (nM) |
| CB3717 | CB3717 | CB3717 | TS | 5.4 |
| EC0284 | CB3717 | CB3717, DAVLBH | MT, TS | 10 |
| EC0347 | None | EC0347 | MT | 1.5 |
| EC2133 | CB3717 | CB3717, tubulysin B | TS | 0.8 |

### Antitumor Activity in KB Tumor Model

Female Balb/c nu/nu mice were fed ad libitum with folate-deficient chow (Harlan diet #TD01013) for the duration of the experiment. KB tumor cells were inoculated subcutaneously at the right flank of each mouse. Mice were dosed after the tumors have reached a range of 107-152 mm³ through the lateral tail vein under sterile conditions in a volume of 200 μL of phosphate-buffered saline (PBS).

Growth of each s.c. tumor was followed by measuring the tumor two times per week. Tumors were measured in two perpendicular directions using Vernier calipers, and their volumes were calculated as 0.5×L×W², where L=measurement of longest axis in mm and W=measurement of axis perpendicular to L in mm.

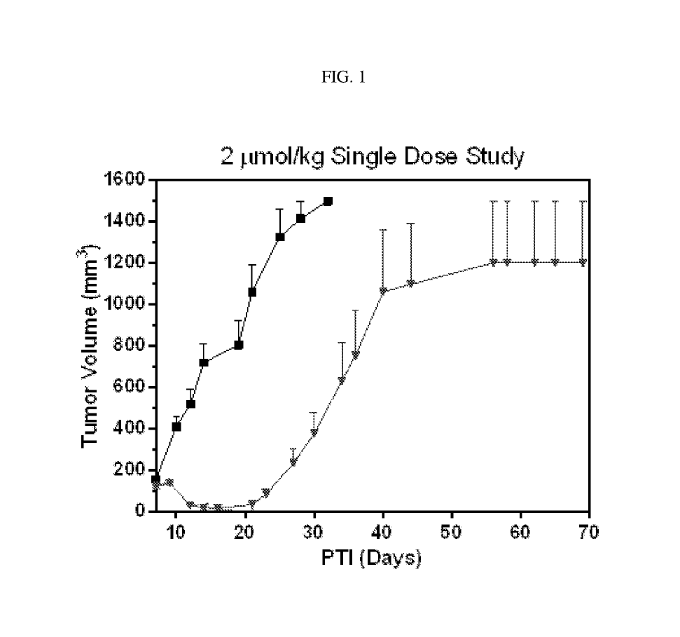

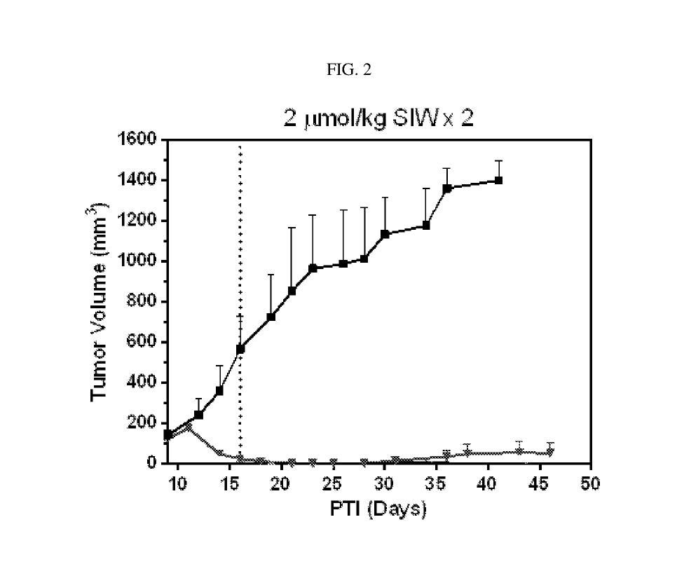

See results in FIG. 1 and FIG. 2.

1. A conjugate of the formula B-L-D¹, wherein B is a binding ligand, L is a linker comprising at least two L¹, at least one AA, and at least one L² of the formula 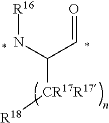
wherein
R¹⁶ is selected from the group consisting of H, D, C₁-C₆ alkyl, C₂-C₆ alkenyl, C₂-C₆ alkynyl, —C(O)R¹⁹, —C(O)OR¹⁹ and —C(O)NR¹⁹R^{19′}, wherein each hydrogen atom in C₁-C₆ alkyl, C₂-C₆ alkenyl and C₂-C₆ alkynyl is independently optionally substituted by halogen, C₁-C₆ alkyl, C₂-C₆ alkenyl, and C₂-C₆ alkynyl, —OR²⁰, —OC(O)R²⁰, —OC(O)NR²⁰R^{20′}, —OS(O)R²⁰, —OS(O)₂R²⁰, —SR²⁰, —S(O)R²⁰, —S(O)₂R²⁰, —S(O)NR²⁰R^{20′}, —S(O)₂NR²⁰R^{20′}, —OS(O)NR²⁰R^{20′}, —OS(O)₂NR²⁰R^{20′}, —NR²⁰R^{20′}, —NR²⁰C(O)R²¹, —NR²⁰C(O)OR²¹, —NR²⁰C(O)NR²¹R^{21′}, —NR²⁰S(O)R²¹, —NR²⁰S(O)₂R²¹, —NR²⁰S(O)NR²¹R^{21′}, —NR²⁰S(O)₂NR²¹R^{21′}, —C(O)R²⁰, —C(O)OR²⁰ or —C(O)NR²⁰R^{20′};
each R¹⁷ and R^{17′} is independently selected from the group consisting of H, D, halogen, C₁-C₆ alkyl, C₂-C₆ alkenyl, C₂-C₆ alkynyl, C₃-C₆ cycloalkyl, 3- to 7-membered heterocycloalkyl, C₆-C₁° aryl, 5- to 7-membered heteroaryl, —OR²², —OC(O)R²², —OC(O)NR²²R^{22′}, —OS(O)R²², —OS(O)₂R²², —SR²², —S(O)R²², —S(O)₂R²², —S(O)NR²²R^{22′}, —S(O)₂NR²²R^{22′}, —OS(O)NR²²R^{22′}, —OS(O)₂NR²²R^{22′}, —NR²²C(O)R²³, —NR²²C(O)OR²³, —NR²²C(O)NR²³R^{23′}, —NR²²S(O)R²³, —NR²²S(O)₂R²³, —NR²²S(O)NR²³R^{23′}, —NR²²S(O)₂NR²³R^{23′}, —C(O)R²², —C(O)OR²², and —C(O)NR²²R^{22′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁° aryl and 5- to 7-membered heteroaryl is independently optionally substituted by halogen, C₁-C₆ alkyl, C₂-C₆ alkenyl, C₂-C₆ alkynyl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′}; or R¹⁷ and R^{17′} may combine to form a C₄-C₆ cycloalkyl or a 4- to 6-membered heterocycle, wherein each hydrogen atom in C₄-C₆ cycloalkyl or 4- to 6-membered heterocycle is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁴, —OC(O)R²⁴, —OC(O)NR²⁴R^{24′}, —OS(O)R²⁴, —OS(O)₂R²⁴, —SR²⁴, —S(O)R²⁴, —S(O)₂R²⁴, —S(O)NR²⁴R^{24′}, —S(O)₂NR²⁴R^{24′}, —OS(O)NR²⁴R^{24′}, —OS(O)₂NR²⁴R^{24′}, —NR²⁴C(O)R²⁵, —NR²⁴C(O)OR²⁵, —NR²⁴C(O)NR²⁵R^{25′}, —NR²⁴S(O)R²⁵, —NR²⁴S(O)₂R²⁵, —NR²⁴S(O)NR²⁵R^{25′}, —NR²⁴S(O)₂NR²⁵R^{25′}, —C(O)R²⁴, —C(O)OR²⁴ or —C(O)NR²⁴R^{24′};
R¹⁸ is selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR²⁶, —OC(O)R²⁶, —OC(O)NR²⁶R^{26′}, —OS(O)R²⁶, —OS(O)₂R²⁶, —SR²⁶, —S(O)R²⁶, —S(O)₂R²⁶, —S(O)NR²⁶R^{26′}, —S(O)₂NR²⁶R^{26′}, —OS(O)NR²⁶R^{26′}, —OS(O)₂NR²⁶R^{26′}, —NR²⁶R^{26′}, —NR²⁶C(O)R²⁷, —NR²⁶C(O)OR²⁷, —NR²⁶C(O)NR²⁷R^{27′}, —NR²⁶C(═NR²⁶″)NR²⁷R^{27′}, —NR²⁶S(O)R²⁷, —NR²⁶S(O)₂R²⁷, —NR²⁶S(O)NR²⁷R^{27′}, —NR²⁶S(O)²NR²⁷R^{27′}, —C(O)R²⁶, —C(O)OR²⁶ and —C(O)NR²⁶R^{26′}, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by halogen, C₁-C₆ alkyl, C₂-C₆ alkenyl, —(CH₂)ₚOR²⁸, —(CH₂)ₚ(OCH₂)_{q}OR²⁸, —(CH₂)ₚ(OCH₂CH₂)_{q}OR²⁸, —OR²⁹, —OC(O)R²⁹, —OC(O)NR²⁹R^{29′}, —OS(O)R²⁹, —OS(O)₂R²⁹, —(CH₂)ₚOS(O)₂OR²⁹, —OS(O)₂OR²⁹, —SR²⁹, —S(O)R²⁹, —S(O)₂R²⁹, —S(O)NR²⁹R^{29′}, —S(O)₂NR²⁹R^{29′}, —OS(O)NR²⁹R^{29′}, —OS(O)₂NR²⁹R^{29′}, —NR²⁹R^{29′}, —NR²⁹C(O)R³⁰, —NR²⁹C(O)OR³⁰, —NR²⁹C(O)NR³⁰R^{30′}, —NR²⁹S(O)R³⁰, —NR²⁹S(O)₂R³⁰, —NR²⁹S(O)NR³⁰R^{30′}, —NR²⁹S(O)₂NR³⁰R^{30′}, —C(O)R²⁹, —C(O)OR²⁹ or —C(O)NR²⁹R^{29′};
each R¹⁹, R^{19′}, R²⁰, R^{20′}, R²¹, R^{21′}, R²², R^{22′}, R²³, R^{23′}, R²⁴, R^{24′}, R²⁵, R^{25′}, R²⁶, R^{26′}, R^{26″}, R²⁹, R^{29′}, R³⁹ and R^{30′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;
R²⁷ and R^{27′} are each independently selected from the group consisting of H, C₁-C₉ alkyl, C₂-C₉ alkenyl, C₂-C₉ alkynyl, C₃-C₆ cycloalkyl, —(CH₂)ₚ(sugar), —(CH₂)ₚ(OCH₂CH₂)_{q}— (sugar) and —(CH₂)ₚ(OCH₂CH₂CH₂)_{q}(sugar);
R²⁸ is H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, 5- to 7-membered heteroaryl or sugar;
n is 1, 2, 3, 4 or 5;
p is 1, 2, 3, 4 or 5;
q is 1, 2, 3, 4 or 5;
L¹ is a releasable linker comprising a disulfide moiety;
D¹ is a drug; and
each * is a covalent bond;
or a pharmaceutically acceptable salt thereof,
with the proviso that the conjugate is not of the formula

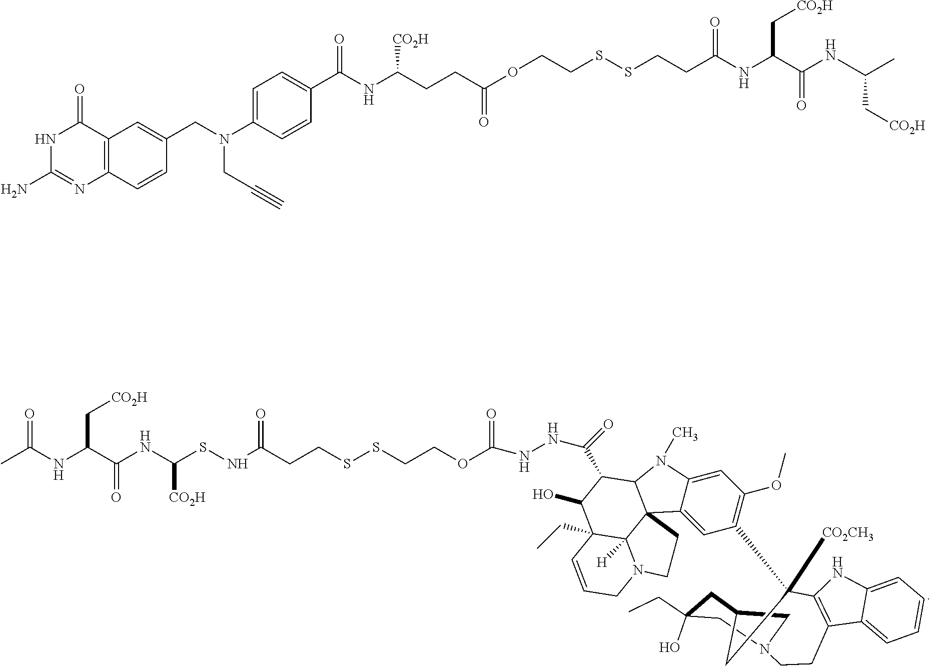

2. The conjugate of claim 1, wherein B is of the formula I 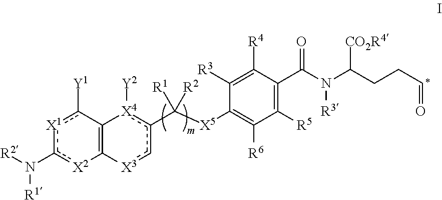
wherein
R¹ and R² in each instance are independently selected from the group consisting of H, D, halogen, C₁-C₆ alkyl, C₂-C₆ alkenyl, C₂-C₆ alkynyl, —OR⁷, —SR⁷ and —NR⁷R^{7′}, wherein each hydrogen atom in C₁-C₆ alkyl, C₂-C₆ alkenyl and C₂-C₆ alkynyl is independently optionally substituted by halogen, —OR⁸, —SR⁸, —NR⁸R^{8′}, —C(O)R⁸, —C(O)OR⁸ or —C(O)NR⁸R^{8′};
R³, R⁴, R⁵ and R⁶ are each independently selected from the group consisting of H, D, halogen, C₁-C₆ alkyl, C₂-C₆ alkenyl, C₂-C₆ alkynyl, —CN, —NO₂, —NCO, —OR⁹, —SR⁹, —NR⁹R^{9′}, —C(O)R⁹, —C(O)OR⁹ and —C(O)NR⁹R^{9′}, wherein each hydrogen atom in C₁-C₆ alkyl, C₂-C₆ alkenyl and C₂-C₆ alkynyl is independently optionally substituted by halogen, OR¹⁰, —SR¹⁰, —NR¹⁰R^{10′}, —C(O)R¹⁰, —C(O)OR¹⁰ or —C(O)NR¹⁰R^{10′};
each R⁷, R^{7′}, R⁸, R^{8′}, R⁹, R^{9′}, R¹⁰ and R^{10′} is independently H, D, C₁-C₆ alkyl, C₂-C₆ alkenyl or C₂-C₆ alkynyl;
X¹ is NR¹¹—, ═N—, —N═, —C(R¹¹)═ or ═C(R¹¹)—;
X² is NR^{11′}- or ═N—;
X³ is NR^{11″}—, —N═ or —C(R^{11′})=;
X⁴ is N═ or C═;
X⁵ is —NR¹²— or —CR¹²R^{12′}—;
Y¹ is H, D, —OR¹³, —SR¹³ or —NR¹³R^{13′} when X¹ is —N═ or —C(R¹¹)═, or Y¹ is ═O when X¹ is NR¹¹—, ═N— or ═C(R¹¹)—;
Y² is H, D, C₁-C₆ alkyl, C₂-C₆ alkenyl, —C(O)R¹⁴, —C(O)OR¹⁴ or —C(O)NR¹⁴R^{14′} when X⁴ is —C═, or Y² is absent when X⁴ is N═;
R^{1′}, R^{2′}, R^{3′}, R^{4′}, R¹¹, R^{11′}, R^{11″}, R¹², R^{12′}, R¹³, R^{13′}, R¹⁴ and R^{14′} are each independently selected from the group consisting of H, D, C₁-C₆ alkyl, C₂-C₆ alkenyl, C₂-C₆ alkynyl, —C(O)R¹⁵, —C(O)OR¹⁵ and —C(O)NR¹⁵R^{15′};
R¹⁵ and R^{15′} are each independently H or C₁-C₆ alkyl; and
m is 1, 2, 3 or 4;
or a pharmaceutically acceptable salt thereof.

3. The conjugate of claim 1 of the formula
B-L¹-L²-AA-L²-AA-L²-L¹-D¹
or a pharmaceutically acceptable salt thereof.

4.-23. (canceled)

24. The conjugate of claim 2, or a pharmaceutically acceptable salt thereof, wherein B is of the formula

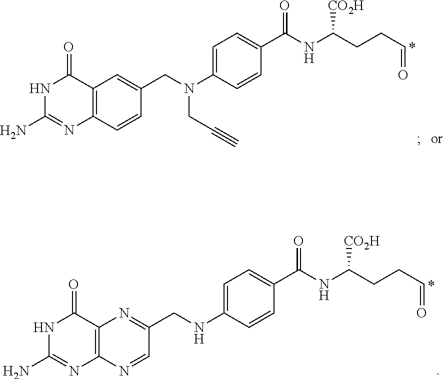 [SMILES: C#CCN(Cc1ccc2nc(N)[nH]c(=O)c2c1)c1ccc(C(=O)N[C@@H](CCC=O)C(=O)O)cc1 ; Nc1nc2ncc(CNc3ccc(C(=O)N[C@@H](CCC=O)C(=O)O)cc3)nc2c(=O)[nH]1]

25. (canceled)

26. The conjugate of claim 1, or a pharmaceutically acceptable salt thereof, wherein at least one AA is in the D-configuration.

27. The conjugate of claim 1, or a pharmaceutically acceptable salt thereof, wherein at least one AA is in the D-configuration.

28. The conjugate of claim 1, or a pharmaceutically acceptable salt thereof, wherein at least one AA is selected from the group consisting of L-asparagine, L-arginine, L-glycine, L-aspartic acid, L-glutamic acid, L-glutamine, L-cysteine, L-alanine, L-valine, L-leucine, L-isoleucine, L-citrulline, D-asparagine, D-arginine, D-glycine, D-aspartic acid, D-glutamic acid, D-glutamine, D-cysteine, D-alanine, D-valine, D-leucine, D-isoleucine and D-citrulline.

29. The conjugate of claim 28, or a pharmaceutically acceptable salt thereof, wherein at least one AA is selected from the group consisting of Asp, Arg, Val, Ala, Cys and Glu.

30. The conjugate of claim 1, wherein each L¹ is selected from the group consisting of 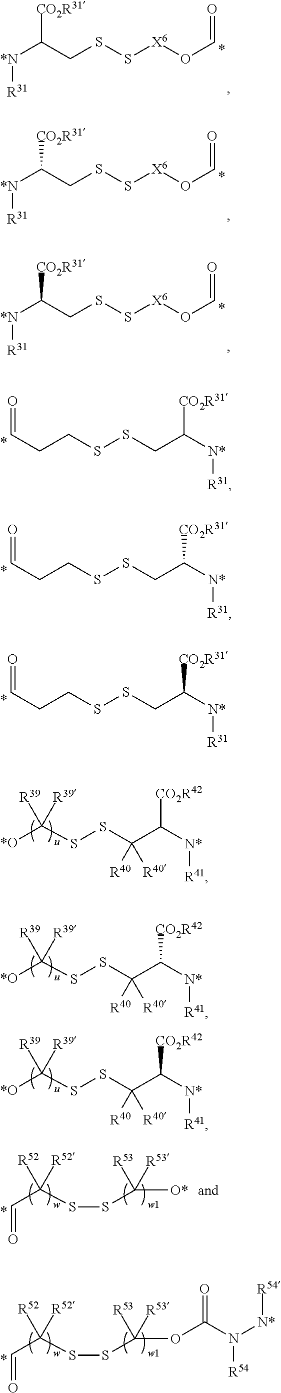
wherein
each R³¹ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³², —OC(O)R³², —OC(O)NR³²R^{32′}, —OS(O)R³², —OS(O)₂R³², —SR³², —S(O)R³², —S(O)₂R³², —S(O)NR³²R^{32′}, —S(O)₂NR³²R^{32′}, —OS(O)NR³²R^{32′}, —OS(O)₂NR³²R^{32′}, —NR³²R^{32′}, —NR³²C(O)R³³, —NR³²C(O)OR³³, —NR³²C(O)NR³³R^{33′}, —NR³²S(O)R³³, —NR³²S(O)₂R³³, —NR³²S(O)NR³³R^{33′}, —NR³²S(O)₂NR³³R^{33′}, —C(O)R³², —C(O)OR³² or —C(O)NR³²R^{32′};
each R^{31′} is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR^{32a}, —OC(O)R^{32a}, —OC(O)NR^{32a}R^{32a′}, —OS(O)R^{32a}, —OS(O)₂R^{32a}, —SR^{32a}, —S(O)R^{32a}, —S(O)₂R^{32a}, —S(O)NR^{32a}R^{32a′}, —S(O)₂NR^{32a}R^{32a′}, —OS(O)NR^{32a}R^{32a′}, —OS(O)₂NR^{32a}R^{32a′}, —NR^{32a}R^{32a′}, —C(O)R^{32a}, —C(O)OR^{32a} or —C(O)NR^{32a}R^{32a′};
each X⁶ is independently C₁-C₆ alkyl or C₆-C₁° aryl(C₁-C₆ alkyl), wherein each hydrogen atom in C₁-C₆ alkyl and C₆-C₁° aryl(C₁-C₆ alkyl) is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR³⁴, —OC(O)R³⁴, —OC(O)NR³⁴R^{34′}, —OS(O)R³⁴, —OS(O)₂R³⁴, —SR³⁴, —S(O)R³⁴, —S(O)₂R³⁴, —S(O)NR³⁴R^{34′}, —S(O)₂NR³⁴R^{34′}, —OS(O)NR³⁴R^{34′}, —OS(O)²NR³⁴R^{34′}, —NR³⁴R^{34′}, —NR³⁴C(O)R³⁵, —NR³⁴C(O)OR³⁵, —NR³⁴C(O)NR³⁵R^{35′}, —NR³⁴S(O)R³⁵, —NR³⁴S(O)₂R³⁵, —NR³⁴S(O)NR³⁵R^{35′}, —NR³⁴S(O)₂NR³⁵R^{35′}, —C(O)R³⁴, —C(O)OR³⁴ or —C(O)NR³⁴R^{34′};
each R^{32a}, R^{32a′}, R³², R^{32′}, R³³, R^{33′}, R³⁴, R^{34′}, R³⁵ and R^{35′} is independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, and 5- to 7-membered heteroaryl;
each R³⁹, R^{39′}, R⁴⁹ and R^{49′} is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁴⁴, —OC(O)R⁴⁴, —OC(O)NR⁴⁴R^{44′}, —OS(O)R⁴⁴, —OS(O)₂R⁴⁴, —SR⁴⁴, —S(O)R⁴⁴, —S(O)₂R⁴⁴, —S(O)NR⁴⁴R^{44′}, —S(O)₂NR⁴⁴R^{44′}, —OS(O)NR⁴⁴R^{44′}, —OS(O)₂NR⁴⁴R^{44′}, —NR⁴⁴C(O)R⁴⁵, —NR⁴⁴C(O)OR⁴⁵, —NR⁴⁴C(O)NR⁴⁵R^{45′}, —NR⁴⁴S(O)R⁴⁵, —NR⁴⁴S(O)₂R⁴⁵, —NR⁴⁴S(O)NR⁴⁵R^{45′}, —NR⁴⁴S(O)₂NR⁴⁵R^{45′}, —C(O)R⁴⁴, —C(O)OR⁴⁴ or —C(O)NR⁴⁴R^{44′};
each R⁴¹ is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁴⁶, —OC(O)R⁴⁶, —OC(O)NR⁴⁶R^{46′}, —OS(O)R⁴⁶, —OS(O)₂R⁴⁶, —SR⁴⁶, —S(O)R⁴⁶, —S(O)₂R⁴⁶, —S(O)NR⁴⁶R^{46′}, —S(O)₂NR⁴⁶R^{46′}, —OS(O)NR⁴⁶R^{46′}, —OS(O)₂NR⁴⁶R^{46′}, —NR⁴⁶C(O)R⁴⁷, —NR⁴⁶C(O)OR⁴⁷, —NR⁴⁶C(O)NR⁴⁷R^{47′}, —NR⁴⁶S(O)R⁴⁷, —NR⁴⁶S(O)₂R⁴⁷, —NR⁴⁶S(O)NR⁴⁷R^{47′}, —NR⁴⁶S(O)₂NR⁴⁷R^{47′}, —C(O)R⁴⁶, —C(O)OR⁴⁶ or —C(O)NR⁴⁶R^{46′};
each R⁴² is independently selected from the group consisting of H, D, C₁-C₆ alkyl, C₂-C₆ alkenyl, C₂-C₇ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl is independently optionally substituted by C₁-C₆ alkyl, C₂-C₆ alkenyl, C₂-C₇ alkynyl, C₃-C₆ cycloalkyl, 3- to 7-membered heterocycloalkyl, C₆-C₁₀ aryl, 5- to 7-membered heteroaryl, —OR⁴³, —OC(O)R⁴³, —OC(O)NR⁴³R^{43′}, —OS(O)R⁴³, —OS(O)₂R⁴³, —SR⁴³, —S(O)R⁴³, —S(O)₂R⁴³, —S(O)NR⁴³R^{43′}, —S(O)₂NR⁴³R^{43′}, —OS(O)NR⁴³R^{43′}, —OS(O)₂NR⁴³R^{43′}, —C(O)R⁴³, —C(O)OR⁴³ or —C(O)NR⁴³R^{43′};
each R⁴³, R^{43′}, R⁴⁴, R^{44′}, R⁴⁵, R^{45′}, R⁴⁶, R^{46′}, R⁴⁷ and R^{47′} is independently selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl;
each R⁵², R^{52′}, R⁵³ and R^{53′} is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁵⁵, —OC(O)R⁵⁵, —OC(O)NR⁵⁵R^{55′}, —OS(O)R⁵⁵, —OS(O)₂R⁵⁵, —SR⁵⁵, —S(O)R⁵⁵, —S(O)₂R⁵⁵, —S(O)NR⁵⁵R^{55′}, —S(O)₂NR⁵⁵R^{55′}, —OS(O)NR⁵⁵R^{55′}, —OS(O)₂NR⁵⁵R^{55′}, —NR⁵⁵C(O)R⁵⁶, —NR⁵⁵C(O)OR⁵⁶, —NR⁵⁵C(O)NR⁵⁶R^{56′}, —NR⁵⁵S(O)R⁵⁶, —NR⁵⁵S(O)₂R⁵⁶, —NR⁵⁵S(O)NR⁵⁶R^{56′}, —NR⁵⁵S(O)₂NR⁵⁶R^{56′}, —C(O)R⁵⁵, —C(O)OR⁵⁵ or —C(O)NR⁵⁵R^{55′};
each R⁵⁴ and R^{54′} is independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR⁵⁷, —OC(O)R⁵⁷, —OC(O)NR⁵⁷R^{57′}, —OS(O)R⁵⁷, —OS(O)₂R⁵⁷, —SR⁵⁷, —S(O)R⁵⁷, —S(O)₂R⁵⁷, —S(O)NR⁵⁷R^{57′}, —S(O)₂NR⁵⁷R^{57′}, —OS(O)NR⁵⁷R^{57′}, —OS(O)₂NR⁵⁷R^{57′}, —NR⁵⁷R^{57′}, —NR⁵⁷C(O)R⁵⁸, —NR⁵⁷C(O)OR⁵⁸, —NR⁵⁷C(O)NR⁵⁸R^{58′}, —NR⁵⁷S(O)R⁵⁸, —NR⁵⁷S(O)₂R⁵⁸, —NR⁵⁷S(O)NR⁵⁸R^{58′}, —NR⁵⁷S(O)₂NR⁵⁸R^{58′}, —C(O)R⁵⁷, —C(O)OR⁵⁷ or —C(O)NR⁵⁷R^{57′};
R⁵⁵, R^{55′}, R⁵⁶, R^{56′} R⁵⁷, R^{57′}, R⁵⁸ and R^{58′} are each independently selected from the group consisting of H, D, C₁-C₇ alkyl, C₂-C₇ alkenyl, C₂-C₇ alkynyl, C₃-C₆ cycloalkyl, 3- to 7-membered heterocycloalkyl, C₆-C₁₀ aryl and 5- to 7-membered heteroaryl;
u is 1, 2, 3 or 4;
w is 1, 2, 3 or 4; and
w1 is 1, 2, 3 or 4;
or a pharmaceutically acceptable salt thereof.

31. (canceled)

32. The conjugate of claim 1, wherein each L² is selected from the group consisting of 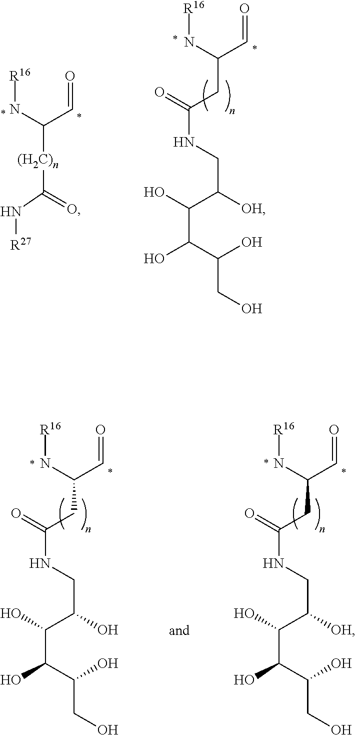
or a pharmaceutically acceptable salt thereof.

33. (canceled)

34. The conjugate of claim 1, or a pharmaceutically acceptable salt thereof, wherein D¹ is a drug selected from the group consisting of a cryptophycin, bortezomib, thiobortezomib, a tubulysin, aminopterin, rapamycin, paclitaxel, docetaxel, doxorubicin, daunorubicin, everolimus, α-amanatin, verucarin, didemnin B, geldanomycin, purvalanol A, ispinesib, budesonide, dasatinib, an epothilone, a maytansine, and a tyrosine kinase inhibitor.

35. The conjugate of claim 1, or a pharmaceutically acceptable salt thereof, wherein D¹ is a tubulysin.

36. The conjugate of claim 1, or a pharmaceutically acceptable salt thereof, wherein D¹ is a tetrapeptide of the formula III 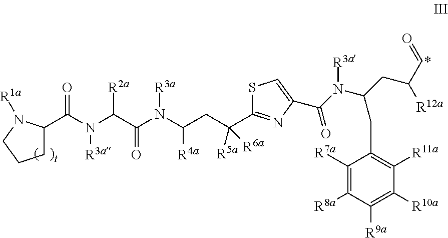
wherein
R^{1a}, R^{3a}, R^{3a′} and R^{3a″} are each independently selected from the group consisting of H, D, C₁-C₆ alkyl, C₂-C₆ alkenyl, C₂-C₆ alkynyl and C₃-C₆ cycloalkyl, wherein each hydrogen atom in C₁-C₆ alkyl, C₂-C₆ alkenyl, C₂-C₆ alkynyl and C₃-C₆ cycloalkyl is independently optionally substituted by halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl, —OR^{13a}, —OC(O)R^{13a}, —OC(O)NR^{13a}R^{13a′}, —OS(O)R^{13a}, —OS(O)₂R^{13a}, —SR^{13a}, —SC(O)R^{13a}, —S(O)R^{13a}, —S(O)₂R^{13a}, —S(O)₂OR^{13a}, —S(O)NR^{13a}R^{13a′}, —S(O)₂NR^{13a}R^{13a′}, —OS(O)NR^{13a}R^{13a′}, —OS(O)₂NR^{13a}R^{13a′}, —NR^{13a}R^{13a′}, —NR^{13a}C(O)R^{14a}, —NR^{13a}C(O)OR^{14a}, —NR^{13a}C(O)NR^{14a}R^{14a′}, —NR^{13a}S(O)R^{14a}, —NR^{13a}S(O)₂R^{14a}, —NR^{13a}S(O)NR^{13a}R^{14a′}, —NR^{13a}S(O)₂NR^{14a}R^{14a′}, —P(O)(OR^{13a})₂, —C(O)R^{13a}, —C(O)OR^{13a} or —C(O)NR^{13a}R^{13a′};
R^{2a}, R^{4a} and R^{12a} are each independently selected from the group consisting of H, D, C₁-C₆ alkyl, C₂-C₆ alkenyl, C₂-C₆ alkynyl;
R^{5a} and R^{6a} are each independently selected from the group consisting of H, D, halogen, C₁-C₆ alkyl, C₂-C₆ alkenyl, C₂-C₆ alkynyl, —SR^{5a}, —OC(O)R^{15a}, —OC(O)NR^{15a}R^{15a′}, and —NR^{15a}R^{15a′}, wherein each hydrogen atom in C₁-C₆ alkyl, C₂-C₆ alkenyl and C₂-C₆ alkynyl is independently optionally substituted by halogen, —OR^{16a}, —SR^{16a}, —NR^{16a}R^{16a′}, —C(O)R^{16a}, —C(O)OR^{16a} or —C(O)NR^{16a}R^{16a′}; or R^{5a} and R^{6a} taken together with the carbon atom to which they are attached form a —C(O)—;
each R^{7a}, R^{8a}, R^{9a}, R^{10a} and R^{11a} is independently selected from the group consisting of H, D, halogen, C₁-C₆ alkyl, C₂-C₆ alkenyl, C₂-C₆ alkynyl, —CN, —NO₂, —NCO, —OR^{17a}, —SR^{17a}, —S(O)₂OR^{17a}, —NR^{17a}R^{17a′}, —P(O)(OR^{17a})₂, —C(O)R^{17a}, —C(O)OR^{17a} and —C(O)NR^{17a}R^{17a′}, wherein each hydrogen atom in C₁-C₆ alkyl, C₂-C₆ alkenyl and C₂-C₆ alkynyl is independently optionally substituted by halogen, —OR^{18a}, —SR^{18a}, —NR^{18a}R^{18a′}, —C(O)R^{18a}, —C(O)OR^{18a} or —C(O)NR^{18a}R^{18a′};
each R^{13a}, R^{13a′}, R^{14a}, R^{14a′}, R^{15a}, R^{15a′}, R^{16a}, R^{16a′}, R^{17a} and R^{17a′} is independently selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl, wherein each hydrogen atom in C₁-C₇ alkyl, C₂-C₇ alkenyl, C₂-C₇ alkynyl, C₃-C₆ cycloalkyl, 3- to 7-membered heterocycloalkyl, C₆-C₁₀ aryl, or 5- to 7-membered heteroaryl is independently optionally substituted by halogen, —OH, —SH, —NH₂ or —CO₂H;
each R^{18a} and R^{18a′} is independently selected from the group consisting of H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 7-membered heteroaryl —C(O)R^{19a}, —P(O)(OR^{19a})₂ and —S(O)₂OR^{19a},
each R¹⁹ is independently selected from H, D, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl and 5- to 7-membered heteroaryl; and
t is 1, 2 or 3.

37.-52. (canceled)

53. The conjugate of claim 36, or a pharmaceutically acceptable salt thereof, wherein D¹ is a tetrapeptide of the formula

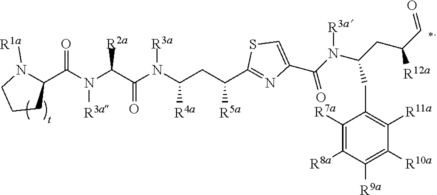

54. The conjugate of claim 36, or a pharmaceutically acceptable salt thereof, wherein D¹ is a tetrapeptide of the formula

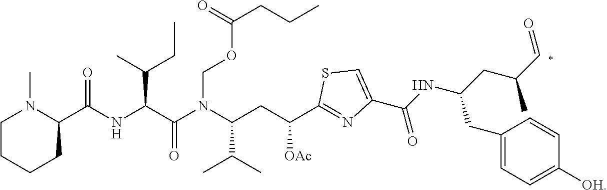 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C=O)cs1)C(C)C]

55. The conjugate of claim 1, or a pharmaceutically acceptable salt thereof, wherein L is of the formula

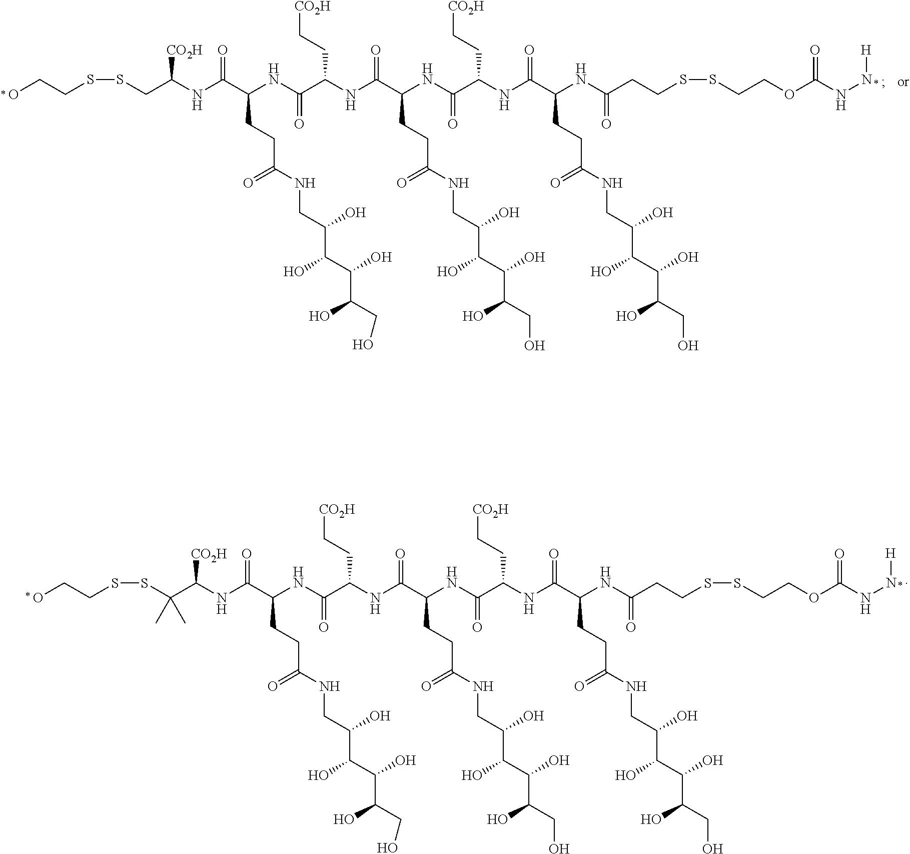

56. (canceled)

58. A pharmaceutical composition comprising a conjugate of claim 1, or a pharmaceutically acceptable salt thereof, and at least one excipient.

59. A method of treating abnormal cell growth in a mammal, including a human, the method comprising administering to the mammal a conjugate of claim 1.

60. The method of claim 59, wherein the abnormal cell growth is cancer

61. The method of claim 60, wherein the cancer is lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In another embodiment of said method, said abnormal cell growth is a benign proliferative disease, including, but not limited to, psoriasis, benign prostatic hypertrophy or restinosis.

62.-63. (canceled)
